SEQUENCE LISTING (1 sequence)

SEQ ID NO: 1 - protein, 168 aa, HOMO SAPIENS
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

(19) United States
(12) Patent Application Publication
(10) Pub. No.: US 2021/0009673 A1
(43) Pub. Date: Jan. 14, 2021

(54) METHODS FOR REGULATING BREAST CANCERS

(71) Applicant: Thomas F. DEUEL, Cambridge, MA (US)

(72) Inventor: Thomas F. DEUEL, Cambridge, MA (US)

(21) Appl. No.: 16/980,807

(22) PCT Filed: Mar. 14, 2019
(86) PCT No.: PCT/US2019/022347

§ 371 Date: Sep. 14, 2020

Related U.S. Application Data
(60) Provisional application No. 62/642,836, filed on Mar. 14, 2018.

(51) Int. Cl. C07K 16/22; C07K 16/30; G01N 33/574; A61P 35/00

(52) U.S. Cl. CPC C07K 16/22; A61P 35/00; G01N 33/57415; C07K 16/3015

(57) ABSTRACT

Disclosed are methods for altering cellular characteristics that pertain to cancer and for slowing, halting, or reversing the transformation of cells to cancerous phenotypes in general or the transformation of cells from more benign forms to less benign forms of cancer, and in particular, breast cancer.

### CROSS REFERENCE TO RELATED APPLICATION

This application claims priority to U.S. App. No. 62/642,836 filed Mar. 14, 2018 which is incorporated herein by reference.

### STATEMENT REGARDING FEDERALLY SPONSORED RESEARCH OR DEVELOPMENT

Not applicable.

### BACKGROUND OF THE INVENTION

The following discussion of the background of the invention is merely provided to aid the reader in understanding the invention and is not admitted to describe or constitute prior art to the present invention.

Cancer is a class of diseases in which a group of cells display uncontrolled growth, invasion, and sometimes metastasis. These three malignant properties of cancers differentiate them from benign tumors, which are self-limited, and do not invade or metastasize. Most cancers form a tumor but some, like leukemia, do not.

Cancer has caused about 13% of all deaths worldwide according to recent surveys. The leading causes include lung cancer, stomach cancer, colorectal cancer, liver cancer, and breast cancer. In the developed world, one in three people will develop some type of cancer during their lifetimes.

Cancers are caused by abnormalities in the genetic material, or expression thereof, of the transformed cells. These abnormalities may be due to the effects of carcinogens, such as tobacco smoke, radiation, chemicals, or infectious agents. Other cancer-promoting genetic abnormalities may randomly occur through errors in DNA replication, or are inherited, and thus present in all cells from birth. The heritability of cancers is usually affected by complex interactions between carcinogens and the host's genome.

Genetic abnormalities found in cancer typically affect two general classes of genes. Cancer-promoting oncogenes are typically activated in cancer cells, giving those cells new properties, such as hyperactive growth and division, protection against programmed cell death, loss of respect for normal tissue boundaries, and the ability to become established in diverse tissue environments. Tumor suppressor genes are then inactivated in cancer cells, resulting in the loss of normal functions in those cells, such as accurate DNA replication, control over the cell cycle, orientation and adhesion within tissues, and interaction with protective cells of the immune system.

Once diagnosed, cancer is usually treated with a combination of surgery, chemotherapy and radiotherapy, to varying effect depending on specific type, location, and stage. While there has been significant progress in the development of targeted therapy drugs that act specifically on detectable molecular abnormalities in certain tumors, and which minimize damage to normal cells, there remains a significant unmet medical need for cancer therapies.

In particular, there remains a significant unmet need for therapies specifically directed to breast cancer.

### SUMMARY OF THE INVENTION

The present invention relates generally to breast cancer. The invention also relates to the altering (e.g., treating preventing, slowing, halting, or reversing) of the transformation of cells from non-cancerous breast tissue cells to cancer cells through any stage or stages of progression including transformation to premalignant cells, transformation to carcinoma in situ in absence of invasion into surrounding tissue and transformation to invasive carcinoma as well as progression of breast cancer cells to a more malignant phenotype. This transformation is intended to include transformation of the cells, from a more benign form to a less benign form. The present invention relates further to pleiotrophin (referenced herein as the protein, PTN or gene Ptn), its direct and downstream targets, as well as the effects of preventing PTN from interacting with those targets. Thus the present invention relates to altering the transformation of cells involved in the development of cancer such as for example breast cancer by altering the PTN/RPTP β/ζ signaling pathway. In various aspects of the invention, this altering of the PTN/RPTP β/ζ signaling pathway may include administering to a breast cancer cell or to a subject having or suspected of having breast cancer, a medicament including an antibody to PTN, a negative PTN, a decoy RPTPβ/ζ or any other substance that decreases the interaction between PTN and RPTPβ/ζ in the cell or in cell of the subject.

### Aspects Including Administering an Effective Amount of Antibody vs Ptn or Fragment Thereof

In a particular aspect, the disclosure provides methods for treating, reducing risk of developing, reversing tumor growth and all other aspects of fighting breast cancer in a subject comprising administering a medicament including an effective amount of an antibody against pleiotrophin (PTN) or a fragment thereof.

### Aspect Comprises Subcategory of Antibodies and Others

In an aspect the disclosure provides methods wherein the antibody against PTN or fragment thereof that is utilized to treat a cancer, cancer cell, tumor, tumor cell, tumor growth or cell in a subject is a monoclonal antibody.

In an aspect the disclosure provides methods wherein the antibody against PTN or fragment thereof that is utilized to treat a cancer, cancer cell, tumor, tumor cell, tumor growth or cell in a subject is a polyclonal antibody.

In an aspect the disclosure provides methods wherein the antibody against PTN or fragment thereof utilized to treat a cancer, cancer cell, tumor, tumor cell, tumor growth or cell in a subject is a humanized antibody.

In an aspect the disclosure provides methods comprising administering an effective amount of a medicament including negative PTN, RPTPβ/ζ to treat a cancer, cancer cell, tumor, tumor cell, tumor growth or cell in a subject rather than, or in combination with, administering an effective amount of an antibody against PTN or a fragment thereof.

### Aspect Involves Multistep/Multiinteractions

In an aspect the disclosure provides methods wherein when an effective amount of antibody such as, for example a medicament including an antibody against PTN is administered to a subject to treat a cancer, cancer cell, tumor, tumor cell, tumor growth or cell, the antibody diminishes the interaction of PTN and RPTP β/ζ, such as by substantially binding to PTN and RPTP β/ζ is consequently no longer substantially inactivated, and wherein when an effective amount of negative PTN is administered to the subject, the negative PTN monomers substantially bind to endogenous PTN monomers to form substantially inactive heterodimers and RPTP β/ζ is consequently no longer substantially inactivated, and wherein when an effective amount of decoy RPTP β/ζ is administered to the subject, one or more dimers of endogenous PTN binds to the decoy RPTP β/ζ and RPTP β/ζ is consequently no longer substantially inactivated.

### BRIEF DESCRIPTION OF THE DRAWINGS

FIG. 1: FIG. 1 illustrates in (A) PTN stimulation of tyrosine phosphorylation of β-catenin in cells that express RPTPβ/ζ and ALK. COS7 cells transiently expressing ALK and RPTPβ/ζ were stimulated with 10 ng PTN/ml. Cell lysates were prepared and used to immunoprecipitate β-catenin. The immunoprecipitates were analyzed in Western blots probed with anti-phosphotyrosine antibodies. Lane 1, cells transfected with ALK that were not stimulated with PTN. Lane 2, cells transfected with ALK stimulated with PTN. Lane 3, cells transfected with empty vector not stimulated with PTN. Lane 4, cells transfected with empty vector stimulated with PTN. In (B), ALK and RPTPβ/ζ, but not Src, are necessary for PTN to stimulate tyrosine phosphorylation of activation of β-catenin. U87MG cells and U87MG cells that had been transfected with an shRNA targeting either RPTPβ/ζ or ALK or preincubated with the src inhibitor PP2 were stimulated with 50 ng PTN/ml or not (control) and cell lysates used to immunoprecipitate β-catenin. The immunoprecipitates were analyzed in Western blots probed with anti-phosphotyrosine antibodies. In (C), PTN does not stimulate tyrosine phosphorylation of Src. U87MG cells were stimulated with 50 ng PTN/ml for 5, 10, and 15 minutes and we used cell lysates in Western blots probed with anti-phospho Src (Y416) and separately with anti-actin antibodies. In (D), EGF stimulates tyrosine phosphorylation of β-catenin in MCF-7 EGFR/RPTPβ/ζ cells that express ALK. MCF-7 cells that stably express the chimeric receptor protein EGFR/RPTPβ/ζ were transfected with ALK and stimulated with EGF for 1 minute, 2 minutes and 5 minutes or pervanadate for 30 minutes. Cell lysates were used to immunoprecipitate β-catenin and the immunoprecipitates were analyzed in Western blots probed with anti-phospho-tyrosine antibodies and reprobed with anti-β-catenin antibodies. In (E), ALK is required for EGF to stimulate increased tyrosine phosphorylation of β-catenin in EGF-stimulated MCF-7 EGFR/RPTPβ/ζ cells. MCF-7 cells that stably express EGFR/RPTPβ/ζ not transfected with ALK were analyzed as above. In (F), PTN and EGF fail to stimulate tyrosine phosphorylation of β-catenin when ALK but not RPTPβ/ζ or EGFR/RPTPβ/ζ are expressed. Lysates of MCF-7 cells and lysates of MCF-7 cells stimulated with EGF were used to immunoprecipitate β-catenin and the immunoprecipitates were analyzed in Western blots probed with anti-phospho-tyrosine antibodies and separately with anti-β-catenin antibodies. Lysates of MCF-7 cells transfected with ALK and stimulated with PTN, EGF or pervanadate were immunoprecipitated with anti-β-catenin antibodies and the immunoprecipitates were analyzed in Western blots probed with anti-phospho-tyrosine antibodies and separately with anti-β-catenin antibodies.

FIG. 2: FIG. 2 illustrates: in (A), ALK and β-catenin associate in vitro. ALK alone, GST-β-catenin alone or ALK incubated together with GST-β-catenin were incubated with glutathione-Agarose beads to isolate GST-tagged β-catenin and proteins associated with GST-β-catenin. The eluates from the glutathione-Agarose beads were analyzed in Western blots probed with anti-ALK antibodies and reprobed with anti-GST antibodies. In (B), time-dependent phosphorylation of β-catenin by ALK in vitro. One microgram of β-catenin was incubated with 20 ng of ALK for 0, 1, 2.5, 5, 10, 15 and 20 minutes. The samples were analyzed in Western blots probed with anti-phospho-tyrosine antibodies and reprobed with anti-GST antibodies. In (C), ALK-dose-dependent phosphorylation of β-catenin in vitro. One microgram of β-catenin was incubated together with 1 ng, 5 ng, 10 ng, 20 ng, 100 ng or 250 ng of ALK for 20 minutes at 37° C. in the in vitro kinase assay with ATP. The samples were analyzed in Western blots probed with anti-phospho-tyrosine antibodies and reprobed with anti-GST antibodies. As negative controls, samples without ALK and without ATP were analyzed. (D) ALK generated by in vitro transcription/translation phosphorylates in vitro β-catenin generated by in vitro transcription/translation. ALK, β-catenin, or both ALK and β-catenin were expressed in an in vitro transcription-translation system and analyzed in Western blots probed with anti-phospho-tyrosine antibodies and reprobed with anti-β-catenin antibodies after incubation An in vitro kinase reaction was then performed and the samples analyzed using. In (E), ALK activated in vivo phosphorylates β-catenin. MCF-7 cells that stably express the chimeric EGFR/RPTPβ/ζ receptor were co-transfected with ALK. Lysates from cells not stimulated (lane 1) or cells stimulated with EGF for 1 minute (lane 2), 5 minutes (lane 3), or with pervanadate (lane 4) were prepared. ALK was immunoprecipitated from lysates and incubated with GST-β-catenin. The samples were analyzed in Western blots probed with anti-phospho-tyrosine antibodies (panels 1 and 3) or with anti-ALK antibodies (panel 2) or anti-GST antibodies (panel 4). In (F), RPTPβ/ζ dephosphorylates β-catenin phosphorylated by ALK in vitro. GST-β-catenin phosphorylated by autoactivated ALK in an in vitro kinase assay was isolated using glutathione-Agarose beads. The precipitates were incubated with GST-RPTPβ/ζ D1 (C1932S) or GST-RPTPβ/ζ D1 and analyzed in Western blots probed with anti-phospho-tyrosine antibodies and reprobed with anti-β-catenin antibodies. In (G), β-catenin tyrosine 333 is the principle tyrosine in β-catenin phosphorylated by ALK. One microgram of recombinant wt GST-β-catenin and GST-β-catenin Y333F were incubated alone or with ALK for 60 minutes at 37° C. in in vitro kinase assays with ATP. The samples were analyzed in Western blots probed with anti-phospho-tyrosine antibodies and reprobed with anti-β-catenin antibodies.

FIG. 3: FIG. 3 illustrates in: (A), Complex of β-catenin with E-cadherin. Beta-catenin shown as a cyan ribbon with a transparent molecular surface in which tyrosine residues highlighted in blue; E-cadherin shown as an alpha carbon trace in purple with phosphoserines shown as atomic stick figures in pink, green and grey. Image created with PMV. The arrow points to phosphoserine 692 and a second arrow points to tyrosine 333. (B) Interaction between phosphoserine 692 of E-cadherin with tyrosine 333 of β-catenin along with two flanking lysine residues (Lys 335 above and Lys 292 below) shown as atomic stick figures. Image created with PMV. (C) The potential interface between a simultaneously phosphorylated tyrosine 333 in β-catenin and a phosphorylated E-cadherin at serine 692.

FIG. 4: FIG. 4 illustrates in: (A), β-catenin phosphorylated by ALK at tyrosine 333 decreases the association of β-catenin with cadherin. Recombinant E-cadherin serine 692 was phosphorylated with CK2 and GSK3β and incubated with recombinant GST-β-catenin, with GST-β-catenin phosphorylated in vitro by ALK, with GST-β-catenin Y333F, or with GST-β-catenin K292A, Y333F, K335A. The GST-conjugated proteins were captured with glutathione-conjugated beads and the phospho-Ser692-E-cadherin that associated with the GST-conjugated proteins was eluted and analyzed in Western blots probed with anti-β-catenin and anti-E-cadherin antibodies. In (B), β-catenin phosphorylated by ALK at tyrosine 333 decreases the association of β-catenin with cadherin. Lysates from U373 cells were prepared and incubated with β-catenin previously incubated with ALK with or without ATP (as control) in vitro. After incubation for 20 minutes at 37° C., β-catenin was then captured with glutathione beads and, after extensive washing, eluted with SDS buffer and analyzed in Western blots with either anti-pan-cadherin antibodies, anti-phospho-tyrosine antibodies, or anti-β-catenin antibodies to determine if β-catenin phosphorylated by ALK at tyrosine 333 associates with cadherin. In (C), Phosphorylation of β-catenin tyrosine 333 by ALK stimulates loss of cell-cell adhesion. The ratio of cells dissociated in calcium-containing media (NTC) vs. the cells dissociated in calcium-free media (NTE) (a “dissociation index,” see Experimental Procedures) was measured. In (D), Phosphorylation of β-catenin by ALK stimulates a morphological transition to a mesenchymal phenotype. MCF10A cells were transfected with vectors encoding PTN and RPTPβ/ζ, PTN and ALK, or PTN, RPTPβ/ζ, and ALK. The morphological appearance was then analyzed 36 hours later and compared with cells transfected with an equal amount of control empty vector.

FIG. 5: FIG. 5 illustrates in: (A), SW-13 and (B), SW-13-Ptn cells observed using phase contrast microscopy. In (C), SW-13 and D, SW-13-Ptn cells were stained using fluorescein-tagged anti-tubulin antibodies (green), phalloidin (red) to visualize F-actin and DAPI (blue) to visualize DNA. The top panel shows the levels of PTN released in the culture media analyzed using Western blots probed with anti-PTN antibodies.

FIG. 6: FIG. 6 illustrates: (A), SW-13 cells (left column) and SW-13-Ptn cells (right column) were used to prepare lysates that were analyzed in Western-blots probed with anti-E-cadherin, anti-N-cadherin, anti-β-catenin, anti-γ-catenin, anti-P120, anti-IQGAP1 and anti-actin antibodies. In (B), cell lysates prepared from SW-13 (left column) and SW-13-Ptn cells were incubated with Rad23-conjugated agarose beads, a protein that binds poly-ubiquitinated proteins, and the precipitates analyzed in Western blots probed with anti-E-cadherin, anti-β-catenin and anti-P120 antibodies.

FIG. 7: FIG. 7 illustrates SW-13 cells (left column) and SW-13-Ptn cells (right column) were stained using fluorescein-tagged anti-E-cadherin, anti-N-cadherin, anti-β-catenin, anti-γ-catenin and anti-P120 antibodies and observed using confocal microscopy. To facilitate the interpretation of the anti-E-cadherin staining DAPI was used to visualize DNA (blue).

FIG. 8: FIG. 8 illustrates: In (A), SW13 and SW13-Ptn cells were plated on 24 well plates coated with different extra-cellular matrix proteins, including collagen type I (0.5 μg/cm²), collagen type IV (1 μg/cm²), fibronectin (5 μg/cm²) and laminin (5 μg/cm²) incubated in DMEM for one hour at 37° C. degrees and 5% CO₂ to sufficiently establish surface adhesion. After one hour cells not adherent were removed and the number of cells attached was counted and represented as cell-ECM adhesion by the mean number of cells attached to a given extracellular matrix after one hour incubation. In (B), RNA from SW-13 and SW-13-Ptn cells was extracted and reverse transcription performed using random hexamers. The cDNA was analyzed using real time PCR to measure the levels of transcription of integrin β1, integrin α1, integrin α2 and integrin α4. The results are shown as relative levels of expression of each gene compared to cyclophylin A in SW-13-Ptn cells in relation to SW-13 cells. In (C), cell lysates prepared from SW-13 cells (left column) and SW-13-Ptn cells (right column) were analyzed in Western-blots probed with anti-integrin α3, anti-integrinβ1, anti-FAK-P-Y-925, anti-integrin α5 and anti-actin antibodies. In (D), SW-13 cells (top panel) and SW-13-Ptn cells (bottom panel) were stained using fluorescein-tagged anti-integrin α3 antibodies and observed using confocal microscopy.

FIG. 9: FIG. 9 illustrates: (A), SW-13 cells (left column) and SW-13-Ptn cells (right column) were stained using fluorescein-tagged anti-cytokeratin 18 and anti-tubulin antibodies and observed using confocal microscopy. In (B), cell lysates prepared from SW-13 cells (left column) and SW-13-Ptn cells (right column) were analyzed in Western-blots probed with anti-tubulin, anti-NF-H/NF-L, anti-keratin 18 and anti-actin antibodies. In (C), RNA from SW-13 and (D), SW-13-Ptn cells was extracted and reverse transcription performed using random hexamers. The cDNA was analyzed using real time PCR to measure the levels of transcription of keratin 2, keratin 5, keratin 6A, keratin 6B, keratin 7, keratin 8, keratin 10, keratin 12, keratin 15, keratin 16, keratin 18, keratin 19 and keratin 20. The results were calculated as relative levels of expression of each gene compared to cyclophylin A and represented and percentage of each individual keratin relative to the total pool of mRNA encoding for those keratins.

FIG. 10: FIG. 10 illustrates: (A), HUVS cells were stimulated with PTN for 2, 5, and 15 minutes. Cell lysates were used to immunoprecipitate EGFR and the immunoprecipitates were analyzed in Western blots probed with anti-phosphotyrosine antibodies. In (B), Lysates from PTN-stimulated HUVS cells that express EGFR were incubated with GST-RPTPβ/ζ D1, GST-RPTPβ/ζ D1 (C1932S), and GST-RPTPβ/ζ D1 (D1900A), a “substrate trap” mutant, which captures the substrate phosphoryl-intermediate in the active site of RPTPβ/ζ with high affinity and specificity. The proteins captured were analyzed in Western blots probed with anti-EGFR antibodies and separately with anti-GST antibodies. In (C), HUVS cells were co-transfected with a full-length EGFR and the vector pC4-Fv1E encoding the Fv domain of FKBP12 needed for enforced dimerization induced by AP20187 (Clackson, Yang et al. 1998) in frame with the intracellular domain of RPTPβ/ζ. AP20187 (2 μM) enforced homodimerization of RPTPβ/ζ and stimulated increase in tyrosine phosphorylation of EGFR 2′, 5′, 10′, and 20′ after stimulation compared with control cells transfected with EGFR alone. (D) A retroviral vector (pSM2) encoding shRNA to “knock down” RPTPβ/ζ was tested in HUVS cells that express endogenous RPTPβ/ζ and EGFR. The cells were stimulated with PTN and cell lysates were used to prepare immunoprecipitates with anti-EGFR antibodies and the immunoprecipitates analyzed in Western blots probed with anti-phosphotyrosine antibodies.

FIG. 11: FIG. 11 illustrates: human umbilical vein stromal cells not stimulated or stimulated with 50 ng/ml of PTN for 2, 5, 10 or 20 minutes were used to prepare cell lysates that were analyzed in Western blots probed with anti-phospho threonine 308 Akt (panel A), anti-phospho serine 473 Akt (panel B), anti-phospho-serine 9 GSK3β (panel C), anti-phospho-serine 33, 37 and threonine 4 β-catenin (panel D) or anti-actin antibodies (panel E). Akt was immunoprecipitated from those lysates using anti-Akt antibodies, incubated with a fusion peptide containing the 20 N-terminal amino acids of GSK3β and analyzed in Western-blots probed using anti-phospho-serine 9 GSK3β antibodies (panel F).

FIG. 12: FIG. 12 illustrates: human umbilical vein stromal cells not stimulated (lane 1) or stimulated with 50 ng/ml of PTN for 15 (lane 2), or 30 minutes (lane 3) were used to prepare cell lysates that were incubated with Rad23-conjugated agarose beads to pull-down ubiquitinated proteins and analyzed in Western-blots probed with anti-β-catenin antibodies (upper panel) and reprobed with anti-ubiquitin antibodies (lower panel). Cells pre-incubated with LY294002 were stimulated with PTN for 15 minutes (lane 4). Cells pre-incubated with lactacystin were stimulated with PTN for 15 minutes (lane 5).

FIG. 13: FIG. 13 illustrates: (A) human umbilical vein stromal cells not stimulated or stimulated for 60 minutes with 1, 10, 25, 50, 100 and 200 ng/ml of PTN were used to isolate their nuclei and prepare lysates that were analyzed in Western blots probed with anti-β-catenin antibodies (upper panel) and anti-Orc-2 antibodies (lower panel). In (B), human umbilical vein stromal cells not stimulated or stimulated with 100 ng/ml of PTN for 5, 15, 30, 60 minutes were used to isolate their nuclei and prepare lysates that were analyzed in Western blots probed with anti-β-catenin antibodies (upper panel) and anti-Orc-2 antibodies (lower panel).

FIG. 14: FIG. 14 illustrates: human umbilical vein stromal cells not stimulated or stimulated for 60 minutes with 100 ng/ml of PTN were stained using fluorescein-tagged anti-β-catenin antibodies and observed using confocal microscopy.

FIG. 15: FIG. 15 illustrates: nuclear fractions of SW-13 and SW-13-Ptn cells were used to prepare lysates that were analyzed in Western-blots probed with anti-β-catenin, anti-γ-catenin, anti-P120 and anti-Orc2 antibodies.

FIG. 16: FIG. 16 illustrates: SW-13 and SW-13-Ptn cells were co-transfected with the plasmids Top-Flash or Fop-flash together with the pSV-β-gal vector. After 24 hours incubation cells were prepared for luciferase activity measurement using the Luciferase Reporter Gene Assay, constant light signal and for β-galactosidase activity measurement using the β-Gal Reporter Gene Assay. Results were expressed as Relative Luciferase Units after subtraction of the Fop-flash background luminescence signal and normalized according to β-galactosidase activity.

FIGS. 17A and 17B: FIGS. 17A and 17B illustrate: RNA from SW-13 and SW-13-Ptn cells was extracted and reverse transcription performed using random hexamers. The cDNA was analyzed using real time PCR to measure the levels of transcription of different genes target of Tcf/Lef. The results are shown as relative levels of expression of each gene compared to cyclophylin A in SW-13-Ptn cells in relation to SW-13 cells.

FIG. 18: FIG. 18 illustrates: that a dominant negative PTN blocks ALK activation. MDA-MB-231 cells were transfected with an empty vector or cDNA encoding a dominant negative PTN 1-40. Cell lysates were prepared and analyzed in Western blots probed with anti-phospho-ALK tyrosine 1586/1604 (left two lanes) or separately with anti-ALK antibodies (right two lanes). Arrows point full length ALK (200 kDa) and activated ALK phosphotyrosines 1586/1604.

FIG. 19: FIG. 19 illustrates: (A) ALK, NPM-ALK, and PTN transcripts are expressed in human breast cancers. We isolated RNA from 46 human breast cancers that was used to prepare cDNAs. The cDNAs were used in PCR with specific primers to detect expression of ALK, NPM-ALK, PTN, and the housekeeping gene GAPDH. The identity of NPM-ALK was confirmed using DNA sequencing of the PCR product that demonstrated the junction between the NPM and ALK genes. In (B), different ALK isoforms are expressed in human breast cancers. Lysates were prepared from 10 human breast cancers and adjacent normal tissues were analyzed in Western blots probed with anti-ALK antibodies. Four major bands of ˜25, ˜50, ˜80, and ˜120 kDa and multiple other bands were identified. In (C), and (D), ALK is activated in human breast cancers. Cell lysates were prepared from 38 human breast cancers (C) and 8 normal breast tissues (D). The lysates were probed with anti-phospho-ALK tyrosine 1586/1604 antibodies and reprobed with anti-actin antibodies. The age and sex of each patient is indicated. The stage, histological phenotype, estrogen and progesterone receptor status, and tumor, node, metastasis staging are also indicated.

FIG. 20: FIG. 20 illustrates: that ALK is activated in different subtypes of human breast cancer. Expression of activated ALK in different human breast cancers is shown. Low level magnification (100×). In (A), infiltrating ductal carcinoma. In (B), infiltrating lobular carcinoma. In (C), Papillary carcinoma. In (D), Medullary adenocarcinoma. In (E), Mucinous carcinoma. In (F), Intraductal carcinoma. In (G), Paget's disease. In (H), normal breast tissue.

FIG. 21: FIG. 21 illustrates: that (A) ALK is ubiquitinated in human breast cancers. Breast cancer cell lysates were prepared and incubated with Rad23-conjugated agarose beads. The proteins captured by Rad23 were analyzed in Western blots probed with anti-ALK antibodies. In (B), NPM-ALK is expressed and activated in human breast cancers. Six human breast cancers were used to prepare cell lysates that were immunoprecipitated using anti-ALK antibodies and analyzed in Western blots probed with anti-NPM, anti-phospho-ALK, and anti-ALK antibodies. Arrow point to an ˜80 kDa protein identified as activated NPM-ALK. In (C), NPM-ALK is expressed in isolated epithelial cells in human breast cancers. Tissue slides of human breast cancers were used to perform fluorescent in situ hybridization with probes that bind telomeric and centromeric sequences immediately upstream and downstream of the ALK gene. Top panel shows one breast cancer cell with two normal chromosomes. Bottom panel shows one breast cancer cell that has one normal chromosome 2 and two copies of chromosome 2 that is translocated. In (D), NPM-ALK is expressed and activated in human breast cancer cell lines. The cell lines MCF-10A, MCF-12A, MCF-7, MDA-MB-231, and T47D were used to prepare lysates that were immunoprecipitated using anti-ALK antibodies and analyzed in Western blots probed with anti-NPM, anti-phospho-ALK, and anti-ALK antibodies.

FIG. 22: FIG. 22 illustrates: in (A), the c-met/ALK dual inhibitor PF2341066 blocks growth of MDA-MB-231 cells. MDA-MB-231 breast cancer cells were treated with 1 nM, 10 nM, 100 nM, and 1 μM PF2341066, the number of viable cells was counted for ten days, and represented as average with standard deviation. In (B), The c-met/ALK dual inhibitor PF2341066 blocks growth of T47D cells. T47D breast cancer cells were treated with 1 nM, 10 nM, 100 nM, and 1 μM PF2341066, the number of viable cells was counted for ten days, and represented as average with standard deviation. In (C), The ALK inhibitor PF2341066 delays MDA-MB-231 cell growth as tumors in flanks of nude mice. MDA-MB-231 cells were injected in flanks of nude mice. The mice were divided in two groups, which were given 50 mg/kg PF2341066 daily by oral gavage or an equal volume of DMSO as control. Tumor size was measured daily and is represented as average tumor burden in each group.

FIG. 23: FIG. 23 illustrates, via western blot: expression of PTN protein in tumor extracts from MMTV-PyMT-Ptn bi-transgenic mice and MMTV-PyMT single transgenic mice.

FIG. 24: FIG. 24 illustrates: in situ hybridization of Ptn mRNA in paraffin sections of tumors from MMTV-PyMT-Ptn and MMTV-PyMT transgenic mice. Antisense RNA probe was used in in situ hybridization of sections of MMTV-PyMT breast cancers. In (A), sections of MMTV-PyMT-Ptn breast cancers. In (B), sections of clustered region of MMTV-PyMT-Ptn breast cancers. In (C), and non-clustered region of MMTV-PyMT-Ptn breast cancers in (D). The Ptn mRNA is most highly expressed in the peri-nodular region of the scirrhous pattern of tumors. Bar=150 um.

FIG. 25: FIG. 25 illustrates: the histological properties of MMTV-PyMT-Ptn breast cancers compared to MMTV-PyMT breast cancers. Panel A and B: reactive stroma surrounding the tumor nodules of breast carcinoma in MMTV-PyMT transgenic mice. H&E stained tumor sections from MMTV-PyMT, in (A), or MMTV-PyMT-Ptn bi-transgenic mice, in (B), were checked by microscopy and representative pictures were shown here. Arrows point to the stromal fibroblast cells surrounding the cancer nodules. Note that there were much more extracellular matrix and stromal cells in breast cancers from MMTV-PyMT-Ptn bi-transgenic mice compared to MMTV-PyMT single transgenic mice. Panel (C and D): Masson Trichrome stained collagen fibers in tumors from MMTV-PyMT in (C) or MMTV-PyMT-Ptn in (D) bi-transgenic mice and representative pictures of invasive nodules were shown here. Arrows point to the thick bundles of collagen fiber that are seen to surround nests of breast cancer cells from MMTV-PyMT-Ptn bi-transgenic mice. Panel (E, F and G): elastic fibers within tumors were stained with resorcin-fuchsin dye. Increased matured elastic fibers wrap the vasculature in breast cancers from MMTV-PyMT-Ptn bitransgenic mice in (E and G) in comparison to MMTV-PyMT single transgenic mice in(F). Elastin fibers also are found in perivascular regions in vessels surrounding the breast cancers from both single and bi-transgenic mice but higher in breast cancers of the bi-transgenes mice. Elastin fibers are also found in regions described as “elastosis” surrounding invasive nodules of tumors from MMTV-PyMT-Ptn bi-transgenic mice (G). Bar=150 μm.

FIGS. 26A and 26B: FIGS. 26A and 26B illustrate: increased activation of MAPK p44/42 and expression of 46-KD Estrogen receptor α (ERα-46) was seen in tumor extracts from MMTV-PyMT-Ptn bi-transgenic mice compared to MMTV-PyMT single transgenic mice.

FIG. 27: FIG. 27 illustrates: expression of ALK in different stages of progression of human prostate adenocarcinomas. In (A), Gleason Score 3+3. In (B), Gleason Score 4+4. In (C), Gleason Score 5+5. In (D), Benign prostate hyperplasia. Note the high levels expression of ALK in tumor associated fibroblasts.

FIG. 28: FIG. 28 illustrates: patterns of expression of ALK in different prostate adenocarcinomas. In (A), accumulation of ALK in nuclei of colon adenocarcinoma cells (Arrows). In (B), paranuclear localization of ALK. In (C), cytoplasmic pattern of expression of ALK. In (D), “dot-like” pattern of expression (Arrows point to some “dots”). Magnification ×600.

FIG. 29: FIG. 29 illustrates: expression of ALK in different human lung cancers. In (A), Squamous cell carcinoma. In (B), Adenocarcinoma. In (C), Large cell carcinoma. In (D), Small cell carcinoma. In (E), Alveolar cell carcinoma. In (F), Normal lung tissue. Magnification ×200.

FIG. 30: FIG. 30 illustrates: subcellular location of ALK in different lung cancers. In (A), Adenocarcinoma, cytoplasmic pattern of expression. In (B), Squamous cell carcinoma, mixed cytoplasmic and nuclear pattern. In (C), Squamous cell carcinoma, nuclear pattern of expression. Arrows point to nuclei with high level of expression of ALK. In (D), Large cell carcinoma, with “dot-like” patterns of expression. Arrows point to accumulation of ALK in “dots”.

FIG. 31: FIG. 31 illustrates: a schematic representation of Receptor Protein Tyrosine Phosphatase (RPTP)β/ζ. □SP—signal peptide, CAH-like-carbonic anhydrase-like domain, Fn-III—fibronectin type III domain containing condroitin sulfate, TM—transmembrane domain, D1—active tyrosine phosphatase domain (C1932—phosphatase catalytic residue), D2—inactive tyrosine phosphatase domain, SLV—C-terminal PDZ binding sequence. Yeast two-hybrid “bait”, residues 1663-2314.

FIG. 32: FIG. 32 illustrates: a schematic representation of HDAC2. Shown is the globular head domain involved in the β-adducin multimer binding, the tail region containing the interactive clone of β-adducin identified in the yeast two-hybrid screen (residues 535-726), the myristoylated alanine-rich C-kinase substrate-like domain (MARCKS), and the protein kinase C sites (residues 713S, 726S), and the single putative tyrosine phosphorylation sites (residue 564Y).

FIG. 33: FIG. 33 illustrates: the identification of human HDAC2 in a yeast two-hybrid screen. Amino acid sequence of the RPTPβ/ζ interactive β-adducin (amino acid 535-726) isolated clone and the α- and γ-adducin isoforms. Residue 564 is the putative tyrosine phosphorylation site in β-adducin and is shown to be conserved in α- and γ-adducin as well as Danio rerio and Gallus gallus adducins. The MARCKS domain in β-adducin derived from the motif in the MARCKS protein (KSPSKKKKKFRTPSFLKKSKKKEKVES) (residue 700-726).

FIG. 34: FIG. 34 illustrates: RPTPβ/ζ D1 domain of capture. Lysates from HeLa cells were incubated with glutathione Sepharose alone (lane 1), GST alone (lane 2), GST-D1 domain of RPTPβ/ζ□ (lane 3), GST-D1 (C1932S) domain of RPTPβ/ζ (lane 4) and GST-D1 (D1932A) domain of RPTPβ/ζ (lane 4). The GST-coupled proteins “captured” from the lysates were probed with an anti-HDAC2 antibodies in Western blots.

FIG. 35: FIG. 35 illustrates: dephosphorylation of HDAC2 from PTN-stimulated HeLa cells by the D1 domain of RPTPβ/ζ. Lysates from HeLa cells not stimulated (lane 1, 2 and 3) and stimulated with PTN (lane 4, 5 and 6) were immunoprecipitated with anti-HDAC2 antibodies and incubated with the RPTPβ/ζ D1 phosphatase active (lane 2 and 5) or RPTPβ/ζ D1 (C1932S) (lane 3 and 6) phosphatase inactive domain of RPTPβ/ζ □in Western-blots probed with an anti-phosphotyrosine antibodies and re-probed with anti-HDAC2 antibodies. Lane 1—HDAC2 is phosphorylated in tyrosine in HeLa cells not stimulated with PTN, Lane 2—HDAC2 in HeLa cells not stimulated with PTN is dephosphorylated by RPTPβ/ζ D1. Lane 3—HDAC2 phosphorylated in tyrosine is not dephosphorylated by the inactivated RPTPβ/ζ D1 (C1932S). Lane 4—The steady state levels of tyrosine phosphorylation of HDAC2 are increased in PTN-stimulated HeLa cells. Lane 5—HDAC2 phosphorylated in tyrosine is dephosphorylated by the phosphatase activity of RPTP β/ζ D1. Lane 6—HDAC2 phosphorylated in tyrosine is not dephosphorylated by the inactivated RPTPβ/ζ D1 (C1932S).

FIG. 36: FIG. 36 illustrates: lysates, prepared from both PTN-stimulated and non-PTN-stimulated HeLa cells, were incubated with GST-RPTPβ/ζ D1. The levels of HDAC2 phosphorylated in tyrosine in lysates from both PTN-stimulated and non-PTN-stimulated cells were markedly reduced when incubated with GST-RPTPβ/ζ D1 (FIG. 6, lanes 2 and 4) compared to lysates not incubated with GST-RPTPβ/ζ D1 (FIG. 6, lanes 1 and 3), demonstrating directly that PTN sharply increases tyrosine phosphorylation of HDAC2 in PTN-stimulated cells, that HDAC2 is phosphorylated in tyrosine in both PTN-stimulated and non-stimulated cells, and that HDAC2 phosphorylated in tyrosine is a substrate of RPTPβ/ζ D1.

FIG. 37: FIG. 37 Illustrates: HDAC activity as related to PTN and RPTPβ/ζ D1

Attachment “B”: Also attached and incorporated into the application is “Attachment B” showing the design for producing an embodiment of the monoclonal antibody of the invention used in certain aspects of the method of the invention. P-1 shows the sequence and target antigen sequences for each of Target Antigen 1 (Positions 33-168 of Human PTN); Target Antigen #2 (Positions 33-96 of Human PTN); and Target Antigen #3 (Positions 101-168). The design called for the monoclonal antibody to bind to one or both of PTN's two domains—its transforming domain (AA's 1-64) and its angiogenic domain (AA's 69-136). Also included in the “Attachment A” are the Phase I ELISA tritration data for each of these targets in sera. Standard protocols known to those skilled in the art of the preparation of the monoclonal antibodies were generally employed in the preparation of the embodiment of the monoclonal antibodies herein disclosed.

### DETAILED DESCRIPTION OF THE INVENTION

The present invention relates to altering the transformation of cells involved in the development of cancer such as, for example, breast cancer by altering the PTN/RPTP β/ζ signaling pathway. In various aspects of the invention, this altering of the PTN/RPTP β/ζ signaling pathway may include administering to a breast cancer cell or to a subject having or suspected of having breast cancer, an antibody to PTN, a negative PTN, a decoy RPTPβ/ζ or any other substance that decreases the interaction between PTN and RPTPβ/ζ in the cell or in cell of the subject.

All publications, patent applications, patents and other references mentioned herein, if not otherwise indicated, are explicitly incorporated by reference herein for all purposes.

Unless otherwise defined, the technical and scientific terms used herein have the same meaning as commonly understood by one of ordinary skill in the art. Expansion and clarification of some terms are provided herein.

As used herein, the term “cancer” refers to cells that exhibit uncontrolled growth. Malignant cancers display uncontrolled growth (division beyond the normal limits), invasion (intrusion on or into and destruction of adjacent tissues), and sometimes metastasis (spread to other locations in the body via lymph or blood). These three properties differentiate them from benign tumors, which are self-limited, and do not invade or metastasize. The term “cancer” can refer to one or more of the following non-limiting examples: adrenocortical carcinoma, AIDS-related cancers, AIDS-related lymphoma, anal cancer, appendix cancer, astrocytoma (childhood), cerebellar or cerebral, basal cell carcinoma, bile duct cancer, extrahepatic (bile duct) cancer, bladder cancer, bone cancer, osteosarcoma/malignant fibrous histiocytoma, brainstem glioma, cerebellar astrocytoma, cerebral astrocytoma/malignant glioma, ependymoma, medulloblastoma, supratentorial primitive neuroectodermal tumors, visual pathway and hypothalamic glioma, breast cancer (e.g., DCIS (Ductal Carcinoma In Situ), LCIS (Lobular Carcinoma In Situ), IDC (Invasive Ductal Carcinoma), e.g., scirrhous carcinoma, less common subtypes of Invasive Ductal Carcinoma (e.g., tubular carcinoma of the breast, medullary carcinoma of the breast, mucinous carcinoma of the breast, papillary carcinoma of the breast, and cribriform carcinoma of the breast), ILC (Invasive Lobular Carcinoma), Paget's Disease of the Nipple, Inflammatory Breast Cancer, Male Breast Cancer, Recurrent and Metastatic Breast Cancer, and phyllodes tumors, e.g., cystosarcoma phyllodes), bronchial adenomas/carcinoids, Burkitt lymphoma, carcinoid tumor (childhood), carcinoid tumor, gastrointestinal, carcinoma of unknown primary, central nervous system lymphoma (primary), cerebellar astrocytoma (childhood), cerebral astrocytoma/Malignant glioma (childhood), cervical cancer, childhood cancers, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor, extragonadal Germ cell tumor, extrahepatic bile duct cancer, eye cancer, intraocular melanoma, eye cancer, retinoblastoma, gallbladder cancer, gastric (stomach) cancer, gastrointestinal carcinoid tumor, gastrointestinal stromal tumor (GIST), germ cell tumor (extracranial), germ cell tumor (extragonadal), germ cell tumor (ovarian), gestational trophoblastic tumor, glioma (adult), glioma (childhood), childhood cerebral astrocytoma, glioma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma (childhood), intraocular melanoma, islet cell carcinoma (endocrine pancreas), Kaposi sarcoma, laryngeal cancer, hairy cell, lip and oral cavity cancer, liver cancer (primary), lung cancer (non-small cell), lung cancer (small cell), lymphomas, lymphoma (AIDS-related), lymphoma (Burkitt), lymphoma (cutaneous T-cell), lymphoma (Hodgkin), lymphomas (non-Hodgkin), lymphoma (primary central nervous system), macroglobulinemia, Waldenström, malignant fibrous histiocytoma of bone/osteosarcoma, medulloblastoma (childhood), melanoma, melanoma (intraocular (eye)), merkel cell carcinoma, mesothelioma (adult), mesothelioma (childhood), metastatic squamous neck cancer with occult primary, mouth cancer, multiple endocrine neoplasia syndrome (childhood), mycosis fungoides, myelodysplastic syndromes, myelodysplastic/myeloproliferative diseases, myelogenous leukemia (chronic), myeloid leukemia (adult acute), myeloid leukemia (childhood acute), myeloma (multiple (cancer of the bone-marrow)), myeloproliferative disorders (chronic), nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma (childhood), salivary gland cancer, sarcoma (soft tissue), sarcoma (uterine), Sézary syndrome, skin cancer (nonmelanoma), skin cancer (melanoma), skin carcinoma, Merkel cell, small intestine cancer, soft tissue sarcoma, squamous cell carcinoma, squamous neck cancer with occult primary, stomach cancer, supratentorial primitive neuroectodermal tumor (childhood), T-cell lymphoma, cutaneous (e.g., mycosis fungoides and Sézary syndrome), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site carcinoma, ureter and renal pelvis, transitional cell cancer, urethral cancer, uterine cancer, endometrial, uterine sarcoma, vaginal cancer, visual pathway and hypothalamic glioma (childhood), vulvar cancer, Waldenström macroglobulinemia, and Wilms tumor (kidney cancer).

In some aspects, “cancer” refers to uncontrolled growth of mesenchymal cells, including one or more of the following examples: adrenocortical carcinoma, AIDS-related cancers, anal cancer, appendix cancer, astrocytoma (childhood), cerebellar or cerebral, basal cell carcinoma, bile duct cancer, extrahepatic (bile duct) cancer, bladder cancer, brainstem glioma, cerebellar astrocytoma, cerebral astrocytoma/malignant glioma, ependymoma, medulloblastoma, supratentorial primitive neuroectodermal tumors, visual pathway and hypothalamic glioma, breast cancer (e.g., DCIS (Ductal Carcinoma In Situ), LCIS (Lobular Carcinoma In Situ), IDC (Invasive Ductal Carcinoma), e.g., scirrhous carcinoma, less common subtypes of Invasive Ductal Carcinoma (e.g., tubular carcinoma of the breast, medullary carcinoma of the breast, mucinous carcinoma of the breast, papillary carcinoma of the breast, and cribriform carcinoma of the breast), ILC (Invasive Lobular Carcinoma), Paget's Disease of the Nipple, Inflammatory Breast Cancer, Male Breast Cancer, Recurrent and Metastatic Breast Cancer), bronchial adenomas/carcinoids, carcinoid tumor (childhood), carcinoid tumor, gastrointestinal, carcinoma of unknown primary, cerebellar astrocytoma (childhood), cerebral astrocytoma/Malignant glioma (childhood), cervical cancer, chronic myeloproliferative disorders, colon cancer, endometrial cancer, ependymoma, esophageal cancer, extracranial germ cell tumor, extragonadal Germ cell tumor, extrahepatic bile duct cancer, eye cancer, intraocular melanoma, eye cancer, retinoblastoma, gallbladder cancer, gastric (stomach) cancer, gastrointestinal carcinoid tumor, germ cell tumor (extracranial), germ cell tumor (extragonadal), germ cell tumor (ovarian), gestational trophoblastic tumor, glioma (adult), glioma (childhood), childhood cerebral astrocytoma, glioma, childhood visual pathway and hypothalamic, gastric carcinoid, head and neck cancer, heart cancer, hepatocellular (liver) cancer, hypopharyngeal cancer, hypothalamic and visual pathway glioma (childhood), intraocular melanoma, islet cell carcinoma (endocrine pancreas), laryngeal cancer, lip and oral cavity cancer, liver cancer (primary), lung cancer (non-small cell), lung cancer (small cell), macroglobulinemia, medulloblastoma (childhood), melanoma, melanoma (intraocular (eye)), merkel cell carcinoma, mesothelioma (adult), mesothelioma (childhood), metastatic squamous neck cancer with occult primary, mouth cancer, multiple endocrine neoplasia syndrome (childhood), nasal cavity and paranasal sinus cancer, nasopharyngeal carcinoma, neuroblastoma, oral cancer, oropharyngeal cancer, ovarian cancer, ovarian epithelial cancer (surface epithelial-stromal tumor), ovarian germ cell tumor, ovarian low malignant potential tumor, pancreatic cancer, pancreatic cancer (islet cell), parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia/multiple myeloma, prostate cancer, rectal cancer, renal cell carcinoma (kidney cancer), renal pelvis and ureter, transitional cell cancer, retinoblastoma, salivary gland cancer, skin cancer (nonmelanoma), skin cancer (melanoma), skin carcinoma, Merkel cell, small intestine cancer, squamous cell carcinoma, squamous neck cancer with occult primary, stomach cancer, supratentorial primitive neuroectodermal tumor (childhood), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site carcinoma, ureter and renal pelvis, transitional cell cancer, urethral cancer, uterine cancer, endometrial, vaginal cancer, visual pathway and hypothalamic glioma (childhood), vulvar cancer, and Wilms tumor (kidney cancer).

As used herein, the term “tumor-associated fibroblast” which may also be referenced as “cancer-associated fibroblast” or “activated fibroblast”, is intended to refer to a fibroblast that is found in the proximity of growing and progressing tumors and the fibroblast acquires properties that promote tumor development and metastasis formation.

As used herein, the term “effective amount” is intended to mean a sufficient amount of a specified component such as, for example an antibody against PTN, to achieve a desired result, such as, for example, the treating of cancer such as, for example breast cancer in a subject.

As used herein, the term “PTN” refers to pleiotrophin which is the 18-kDa protein encoded by the Ptn gene. PTN is a cytokine that signals diverse functions, including those of a differentiation factor/growth factor/angiogenic factor for various cell types (for review, see Muramatsu, T. (2002) Midkine and pleiotrophin: two related proteins involved in development, survival, inflammation and tumorigenesis. J. Biochem. (Tokyo) 132, 359-371). PTN has a high affinity for heparin and is also known as heparin-binding brain mitogen (HBBM) or heparin-binding growth factor 8 (HBGF-8) or neurite growth-promoting factor 1 (NEGF1) or heparin affinity regulatory peptide (HARP) or heparin binding growth associated molecule (HB-GAM). Reference to PTN is intended to include wild-type PTN as well as mutant forms of PTN that are capable of binding to RPTP β/ξ and eliciting a dimerization of RPTP β/ξ and a subsequent inactivation of the tyrosine phosphatase activity of RPTP β/ξ

As used herein, the term “RPTP β/ξ” or “RPTP BETA/ZETA” or “RPTP B/Z” refers to Receptor Protein Tyrosine Phosphatase β/ζ. RPTP β/ξ is a transmembrane protein having a cell surface receptor portion and a cytosoplasmic portion having protein tyrosine phosphatase activity. PTN is the natural ligand for RPTP β/ξ and upon PTN binding to RPTP β/ξ, it elicits a dimerization of RPTP β/ξ and a subsequent inactivation of the tyrosine phosphatase activity of RPTP β/ξ. The term “activity of RPTPβξ” as used herein includes the catalytic activity of RPTPβξ.

As used herein, the term “ALK” refers to Anaplastic Lymphoma Kinase. ALK is a receptor protein tyrosine kinase of the insulin receptor superfamily known to have an essential role in normal development. The term “activity of ALK” as used herein includes the catalytic activity of ALK.

As used herein, the term “Akt” refers to the serine/threonine protein kinase also known as Protein Kinase B or RAC-PK, that plays a key role in multiple cellular processes including glucose metabolism, cell proliferation, apoptosis, transcription and cell migration.

As used herein, the term “GSK3β” refers to Glycogen Synthase Kinase 33. GSK 3β is a serine/threonine kinase that is thought to regulate many biological functions, such as embryonic development, metabolism, tumorigenesis, and cell death, by regulation of many intracellular signaling pathways through phosphorylation of substrates.

As used herein, the term “protein kinase C” or “PKC” refers to family of protein kinase enzymes that are involved in controlling the function of other proteins through the phosphorylation of hydroxyl groups of serine and threonine amino acid residues on these proteins. The term includes members of the family of isomers of protein kinase C.

As used herein the term “E-cadherin” refers to epithelial cadherin also known as cadherin-1, CAM 120/80 or uvomorulin. In humans, E-cadherin is encoded by the CDH1 gene. The E-cadherin is a calcium-dependent cell-cell adhesion glycoprotein composed of five extracellular cadherin repeats, a transmembrane region, and a highly conserved cytoplasmic tail. In the phosphorylated state, the phosphoserine-692 of E-cadherin associates with tyrosine-333 of β-catenin by hydrogen bonding to form a β-catenin/E-cadherin complex in a cell

As used herein the term “β-catenin” refers to the 88 kDa protein that in humans is encoded by the CTNNB1 gene. β-catenin regulate cell growth and adhesion between cells. The tyrosine-333 of β-catenin-tyrosine associates with the phosphoserine-692 of E-cadherin by hydrogen bonding to form a β-catenin/E-cadherin complex in a cell

As used herein, the term “β-catenin/E-cadherin complex” is intended to refer to the complex formed by hydrogen bonding of β-catenin-tyrosine-333 with the phosphoserine 692 of E-cadherin.

Reference herein to activity of a substance may include catalytic activity of the substance.

As used herein, the term “expression” with respect for a substance such as, for example PTN, RPTPβξ or ALK, is intended to include expression of either or both of the protein and gene.

In various aspects, the present invention involves administering to a breast cancer cell or to a subject having or suspected of having breast cancer, an antibody to PTN, a negative PTN, a decoy RPTPβ/ζ or any other substance that decreases the interaction between PTN and RPTPβ/ζ in the cell or in cell of the subject.

As used herein, the term “antibody” refers to an immunoglobulin molecule that reacts with a specific antigen. Antibodies contain a complementarity determining region (CDR) with a unique amino acid structure that specifically binds to an epitope on the antigen. Antibodies can be polyclonal antibodies that differ in their epitope binding and complementarity region amino acid sequence, however share overall target specificity. Alternatively, antibodies can be monoclonal antibodies having a singular epitope specificity originally produced by one B-cell and sharing identical sequence. Antibodies can include non-human antibodies (e.g. antibodies obtained upon immunizing an animal such as mouse, guinea pig, rabbit or rat), chimaeric or humanized antibodies in which non-human CDR coding regions responsible for the desired binding properties are inserted into a human antibody “scaffold”, or fully human antibodies such as are produced for example, using transgenic mouse or phage display techniques.

As used herein, the term “an antibody against PTN or a fragment thereof” is intended to include either or both of an antibody or fragment of an antibody and PTN or a fragment of PTN so that the term can include “an antibody or fragment thereof against PTN or a fragment of PTN”.

As used herein, a fragment of an antibody such as a fragment of an antibody against PTN includes that portion of an intact antibody that binds to the antigen, for example, the antigen binding variable region. Such antibody fragments can include Fv, Fab, Fab′, F(ab′), F(ab′)₂, Fv fragment or the like. The antigen-binding antibody fragments can also include binding-domain containing immunoglobin fusion proteins.

As used herein, a fragment of PTN refers to a polypeptide portion of PTN that contains at least one epitope of the PTN.

As used herein, the term “negative pleiotrophin” or “negative PTN” refers to a portion of PTN capable of dimerizing with a wild type PTN monomer but incapable of conferring wild type dimer activity. Thus, negative PTN monomers act as a sink for wild type monomers, thereby decreasing the concentration or amount of active PTN dimers. In some aspects, negative PTN can be synthetically or recombinantly produced and purified. In general, the negative PTN monomers substantially bind to endogenous PTN monomers to form substantially inactive heterodimers such that RPTPβ/ζ is consequently no longer substantially inactivated and ALK is consequently substantially dephosphorylated by RPTP β/ζ, The negative PTN may be a fragment of PTN, a mutant form of PTN or any other peptide or substance that decreases the interaction of PTN and RPTPβ/ζ. Dominant negative PTN (PTN 1-40) refers to mutant PTN (residues 1-40) which functions as a dominant negative by forming mixed disulfide linked heterodimers with wild-type PTN.

As used herein, the term “decoy RPTP β/ζ” refers to a monomeric or dimeric, inactive RPTP β/ζ. In some aspects, decoy RPTP β/ζ can be any portion of RPTP β/ζ that is recognized by PTN but does not possess wild type RPTP β/ζ activity, synthetically or recombinantly produced and purified. Decoy RPTP β/ζ acts as a sink for PTN and prevents its interaction with wild type RPTP β/ζ. As such, decoy RPTPβ/ζ binds to one or more dimmers of endogenous PTN such that RPTP β/ζ is consequently no longer substantially inactivated and ALK is consequently substantially dephosphorylated by RPTP β/ζ. The decoy RPTPβ/ζ may be exogenous RPTPβ/ζ, a fragment of RPTPβ/ζ, a mutant form of RPTPβ/ζ or any other substance binds to PTN dimmers and decreases the binding of endogenous PTN to endogenous RPTPβ/ζ.

The present invention includes aspects as described in each of the aspects 1-211 below, in which the methods include administering and/or selecting an antibody against PTN or a fragment thereof, a negative PTN, a decoy RPTPβ/ζ or any combination of two or more thereof.

As used herein, the term “uncomplexed PTN” refers to PTN in monomeric or homodimeric form, “free”, or unbound to other macromolecules, for example, RPTP β/ζ. An antibody to PTN binds to uncomplexed PTN and removes it from the population of PTN capable to binding to other substrates like RPTP β/ζ.

As used herein, the term “uncomplexed RPTP β/ζ” or “uncomplexed monomeric RPTP β/ζ” refers to RPTP β/ζ that is not bound by PTN.

As used herein, the term “(PTN)₂-(RPTP β/ζ)₂ heterotetramer” refers to a dimer of PTN bound to a dimer of RPTP β/ζ.

As used herein, the term “phosphorylation state” refers to the average amount or average concentration of phosphates on sites in proteins that can be phosphorylated. As one non-limiting example, consider a protein with three sites capable of being phosphorylated (either by a kinase or via autophosphorylation). If the steady state, or normal, level of phosphorylation for the example protein in the cell is for one site to be phosphorylated (i.e., an average of one phosphorylation across all molecules of the example protein), an increase in phosphorylation state would be represented by the average phosphorylation across all molecules of example protein to be greater than one. A decrease in phosphorylation state would be represented by the average phosphorylation across all molecules of example protein to be less than one.

As used herein, the term “EMT” refers to the epithelial-mesenchymal transition, which is characterized by a loss of epithelial cell-cell junctions and the polarized epithelial phenotype, which, in turn, leads to a more motile fibroblast-like cellular phenotype.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “substantially bind” or “substantially binds” refers, in some aspects, to greater than about 0.1% of an antibody or fragment thereof, negative PTN or decoy RPTPβ/ζ binding to its specific partner. In other aspects, the terms refer to greater than about 1% of an antibody or fragment thereof, negative PTN or decoy RPTPβ/ζ binding to its specific partner. In other aspects, the terms refer to greater than about 2% of an antibody or fragment thereof, negative PTN or decoy RPTPβ/ζ binding to its specific partner. In other aspects, the terms refer to greater than about 5% of an antibody or fragment thereof, negative PTN or decoy RPTPβ/ζ binding to its specific partner. In other aspects, the terms refer to greater than about 10% of an antibody or fragment thereof, negative PTN or decoy RPTPβ/ζ binding to its specific partner. In other aspects, the terms refer to greater than about 15% of an antibody or fragment thereof, negative PTN or decoy RPTPβ/ζ binding to its specific partner. In other aspects, the terms refer to greater than about 20% of an antibody or fragment thereof, negative PTN or decoy RPTPβ/ζ binding to its specific partner. In other aspects, the terms refer to greater than about 25% of an antibody or fragment thereof, negative PTN or decoy RPTPβ/ζ binding to its specific partner. In other aspects, the terms refer to greater than about 30% of an antibody or fragment thereof, negative PTN or decoy RPTPβ/ζ binding to its specific partner. In other aspects, the terms refer to greater than about 35% of an antibody or fragment thereof, negative PTN or decoy RPTPβ/ζ binding to its specific partner. In other aspects, the terms refer to greater than about 40% of an antibody or fragment thereof, negative PTN or decoy RPTPβ/ζ binding to its specific partner. In other aspects, the terms refer to greater than about 45% of an antibody or fragment thereof, negative PTN or decoy RPTPβ/ζ binding to its specific partner. In other aspects, the terms refer to greater than about 50% of an antibody or fragment thereof, negative PTN or decoy RPTPβ/ζ binding to its specific partner. In other aspects, the terms refer to greater than about 60% of an antibody or fragment thereof, negative PTN or decoy RPTPβ/ζ binding to its specific partner. In other aspects, the terms refer to greater than about 70% of an antibody or fragment thereof, negative PTN or decoy RPTPβ/ζ binding to its specific partner. In other aspects, the terms refer to greater than about 80% of an antibody or fragment thereof, negative PTN or decoy RPTPβ/ζ binding to its specific partner. In other aspects, the terms refer to greater than about 90% of an antibody or fragment thereof, negative PTN or decoy RPTPβ/ζ binding to its specific partner. The specific partner for PTN antibody or fragment thereof is PTN. The specific partner for negative PTN is wild type PTN. The specific partner for decoy RPTPβ/ζ is PTN.

As used herein, the term “substantially associated” such as, for example in reference to hydrogen bonding of the β-catenin/E-cadherin complex, is intended to mean that greater than about 50% (about 0.5 fold), greater than about 60% (about 0.6 fold), greater than about 70% (about 0.7 fold), greater than about 80% (about 0.8 fold), greater than about 90% (about 0.9 fold) or more of the β-catenin and/or E-cadherin are in the complexed form. Similarly, the term “substantially dissociated” is intended to mean that greater than about 50% (about 0.5 fold), greater than about 60% (about 0.6 fold), greater than about 70% (about 0.7 fold), greater than about 80% (about 0.8 fold), greater than about 90% (about 0.9 fold) or more of the β-catenin and/or E-cadherin are in the dissociated form. As used herein, the term “association state” or “dissociation state” of the β-catenin/E-cadherin complex refers to the degree of association or dissociation of the complex as the case may be. Thus, the dissociation state of the β-catenin-E-cadherin complex may be such that about 10% (about 0.1 fold), about 20% (about 0.2 fold), about 30% (about 0.3 fold), about 40% (about 0.4 fold), about 50% (about 0.5 fold), about 60% (about 0.6 fold), about 70% (about 0.7 fold), about 80% (about 0.8 fold), about 90% (about 0.9 fold) or more of the β-catenin and/or E-cadherin is in the dissociated form and the association state of the β-catenin-E-cadherin complex may be such that about 10% (about 0.1 fold), about 20% (about 0.2 fold), about 30% (about 0.3 fold), about 40% (about 0.4 fold), about 50% (about 0.5 fold), about 60% (about 0.6 fold), about 70% (about 0.7 fold), about 80% (about 0.8 fold), about 90% (about 0.9 fold)or more of the β-catenin and/or E-cadherin is in the associated form.

As used herein, the term “substantially activate” or “substantially activated” refers to increasing the activity of a molecule, enzyme, receptor, biochemical pathway, etc. As an example, substantially activating RPTPβ/ζ, a tyrosine phosphatase, results in increasing its phosphatase activity. In some aspects, the terms refer to activating or increasing activity by more than about 2000% (about 20 fold), more than about 1500% (about 15 fold), more than about 1000% (about 10 fold), more than about 500% (about 5 fold), more than about 400% (about 4 fold) by more than about 300% (about 3 fold) by more than about 200% (about 2 fold) by more than about 100% (about fold), more than about 90% (about 0.9 fold), more than about 80% (about 0.8 fold), more than about 70% (about 0.7 fold), more than about 60% (about 0.6 fold) by more than about 50% (about 0.5 fold) by more than about 40% (about 0.4 fold) by more than about 30% (about 0.3 fold) by more than about 20% (about 0.2 fold), more than about 10% (about 0.1 fold) or more than about 5% (about 0.05 fold).

As used herein, the term “substantially dephosphorylate” or “substantially dephosphorylated” refers, in some aspects, to a decrease in phosphorylation state of more than about 90% (about 0.9 fold), more than about 80% (about 0.8 fold), more than about 70% (about 0.7 fold), more than about 60% (about 0.6 fold), more than about 50% (about 0.5 fold), more than about 40% (about 0.4 fold),more than about 30% (about 0.3 fold),more than about 20% (about 0.2 fold), more than about 10% (about 0.1 fold), more than about 5% (about 0.05 fold), more than about 1% (about 0.01 fold) or more than about 0.1% (about 0.01 fold).

As used herein, the term “substantially phosphorylate” or “substantially phosphorylated” refers, in some aspects, to an increase in phosphorylation state of more than about 90% (about 0.9 fold), more than about 80% (about 0.8 fold), more than about 70% (about 0.7 fold), more than about 60% (about 0.6 fold), more than about 50% (about 0.5 fold), more than about 40% (0.4 fold), more than about 30% (0.3 fold), more than about 20% (about 0.2 fold), more than about 10% (about 0.1 fold) or more than about 5% (about 0.05 fold).

As used herein, the expression “substantially in the form of β-catenin-phosphotyrosine-333” is intended to mean that greater than about 50% (about 0.5 fold), greater than about 60% (about 0.6 fold), greater than about 70% (about 0.7 fold), greater than about 80% (about 0.8 fold), greater than about 90% (about 0.9 fold) or more of the β-catenin is in the form of β-catenin-phosphotyrosine-333.

As used herein, the term “constitutively expresses PTN” refers to expression of PTN by a cell wherein the expression is not substantially affected by the cell cycle or other regulatory processes, i.e., unregulated expression.

As used herein, the term “substantially deactivate” or “substantially deactivated” refers, in some aspects, to decreasing the activity of a molecule, enzyme, receptor, biochemical pathway, etc. As an example, substantially deactivating RPTPβ/ζ, a tyrosine phosphatase, results in decreasing its phosphatase activity. In various aspects, the terms refer to decreasing activity by more than about 5%, more than about 10%, more than about 20%, more than about 30%, more than about 40%, more than about 50%, more than about 60%, more than about 70%, more than about 80%, more than about 90%, more than about 95%, more than about 99% or more than about 99.9%. In various aspects, the terms refer to decreasing activity values to less than about 0.95 fold, less than about 0.90 fold, less than about 0.80 fold, less than about 0.70 fold, less than about 0.60 fold, less than about 0.50 fold, less than about 0.40 fold, less than about 0.30 fold, less than about 0.20 fold, less than about 0.10 fold, less than about 0.05 fold, less than about 0.01 fold or less than about 0.001 fold of that prior to the decrease.

As used herein, the term “molecular targets of ALK” refers to any molecule that ALK interacts with specifically, for example, via molecular recognition as opposed to non-specific, low affinity interaction.

As used herein, the term “deposition of collagen” and “deposition of elastin” by a cell, a cancer cell or a tumor cell or a tumor-associated fibroblast refers to the extracellular deposit of collagen and/or elastin.

In a general sense, the disclosure provides methods for modification of, prevention of and/or treatment of cancers and cells that are precancerous.

### Aspects of the Invention

The disclosure provides various aspects of the invention including the following:

Aspect 1 In an aspect, the disclosure provides a method for treating cancer in a subject comprising administering a medicament including an effective amount of an antibody against PTN or a fragment thereof, to a subject in need thereof.

Aspect 2 In another aspect, the disclosure provides a method for treating cancer in a subject in need thereof comprising a) administering a medicament including an amount of an antibody against PTN or a fragment thereof, to the subject in need thereof and b) determining the association or dissociation state of the β-catenin/E-cadherin complex in a cell of the subject, wherein the amount of antibody administered is increased if the β-catenin/E-cadherin complex is substantially dissociated.

Aspect 3 In another aspect, the disclosure provides a method for treating cancer in a subject in need thereof comprising a) administering a medicament including an amount of an antibody against PTN or a fragment thereof, to the subject in need thereof and b) measuring the level of β-catenin and/or β-catenin-phosphotyrosine-333 in a cell of the subject; wherein if β-catenin is substantially in the form of β-catenin-phosphotyrosine-333 the amount of antibody administered is increased.

Aspect 4 In another aspect, the disclosure provides a method for treating cancer in a subject in need thereof comprising a) selecting an antibody against PTN on the basis of the antibody being capable of substantially increasing the association of the β-catenin-tyrosine-333 with E-cadherin phosphoserine-692 to form β-catenin/E-cadherin complex in a cell of the subject and b) administering an effective amount of a medicament including the antibody to the subject, wherein the complex is substantially dissociated in the cell prior to administration.

Aspect 5 In another aspect, the disclosure provides a method for treating cancer in a subject in need thereof comprising (a) selecting an antibody against PTN on the basis of the antibody being capable of modulating the RPTP β/ζ signaling pathway in a cell of the subject comprising (i) substantially decreasing binding of PTN to RPTP β/ζ such that (ii) RPTP β/ζ is consequently no longer substantially inactivated such that (iii) ALK is consequently substantially dephosphorylated by RPTP β/ζ such that (iv) dephosphorylated ALK fails to substantially phosphorylate tyrosine-333 of β-catenin such that (v) association of β-catenin with E-cadherin by hydrogen bonding between tyrosine-333 of β-catenin and phosphoserine-692 of E-cadherin is no longer substantially diminished such that (iv) loss of cell-cell adhesion is no longer substantially diminished such that (v) epithelial-mesenchymal transition is no longer substantially elicited; and (b) administering a medicament including an effective amount of the antibody to the subject in need thereof.

Aspect 6 In another aspect, the disclosure provides a method for treating cancer in a subject in need thereof comprising a) selecting an antibody against PTN on the basis of the antibody being capable of decreasing phosphorylation of tyrosine-333 of β-catenin upon the antibody binding to PTN in a cell of the subject in need thereof and b) administering a medicament including an effective amount of the antibody to the subject.

Aspect 7 In another aspect, the disclosure provides a method of cancer diagnosis in a subject, the method comprising determining the phosphorylation state of tyrosine-333 of β-catenin in a cell suspected of being cancerous, wherein the cell is obtained from the subject and wherein the cell is determined to be cancerous if tyrosine-333 of β-catenin is substantially phosphorylated in the cell.

Aspect 8 In another aspect, the disclosure provides a method of cancer diagnosis in a subject, the method comprising determining the dissociation and/or association state of the β-catenin-tyrosine-333-E-cadherin-phosphoserine-692 complex in a cell suspected of being cancerous wherein the cell is obtained from the subject and wherein the cell is determined to be cancerous if the β-catenin-tyrosine-333-E-cadherin-phosphoserine-692 complex is substantially dissociated in the cell.

Aspect 9 In another aspect, the disclosure provides a method of identifying an anti-cancer compound, the method comprising a) providing a cell in which β-catenin-tyrosine-333 is substantially phosphorylated, b) administering a candidate compound to the cell, c) measuring the phosphorylation state of β-catenin-tyrosine-333 of the cell and d) determining that the candidate compound is an anti-cancer compound if the phosphorylation state of β-catenin-tyrosine-333 in the cell is decreased in the presence of the compound.

Aspect 10 In another aspect, the disclosure provides a method of identifying an anti-cancer compound, the method comprising a) providing a cell in which the β-catenin/E-cadherin complex is substantially dissociated, b) administering a candidate compound to the cell, c) measuring the dissociation state or the association state of the β-catenin/E-cadherin complex in the cell and d) determining that the candidate compound is an anti-cancer compound if the dissociation state of the β-catenin/E-cadherin complex is decreased or the association state of the β-catenin/E-cadherin complex is increased in the presence of the compound.

Aspect 11 In another aspect, the disclosure provides a method for reducing risk of developing cancer, a method for reducing cancer cell proliferation, a method for reversing tumor growth, a method for reducing cancer cell invasiveness, a method for reducing cancer cell motility, a method for reducing tumor cell angiogenesis, a method for treating a cell that expresses PTN, RPTPβ/ζ and ALK, a method for treating a cell that constitutively expresses PTN, a method for reducing the concentration of uncomplexed PTN in a cell, a method for increasing the concentration of uncomplexed monomeric RPTPβ/ζ in a cell, a method for decreasing the concentration of (PTN)2-(RPTP β/ζ)2 heterotetramer in a cell, a method for substantially reducing activity of ALK in a cell, a method for reducing the activity of ALK in a cell, a method for reducing the phosphorylation state of ALK in a cell, a method for inhibiting tumor growth, a method for increasing effectiveness of anticancer treatment of a cancer cell including in administering radiation therapy, a method for diminishing the likelihood of development of a cancer, a method for reducing phosphorylation of tyrosine-333 of β-catenin in a cell in a subject, comprising administering an effective amount of an antibody against PTN or a fragment thereof, or a medicament containing same, to a subject in need thereof.

Aspect 12 In another aspect, the disclosure provides a method of aspect 10 or 11 wherein when an effective amount of antibody against PTN is administered to the subject, the antibody substantially binds to PTN, and wherein RPTP β/ζ is consequently no longer substantially inactivated, and wherein ALK is consequently substantially dephosphorylated by RPTP β/ζ, and wherein dephosphorylated ALK fails to substantially phosphorylate tyrosine-333.

Aspect 13 In another aspect, the disclosure provides a method for increasing dephosphorylation of tyrosine-333 of β-catenin in a cell of a subject comprising administering a medicament including an effective amount of an antibody against PTN or a fragment thereof, to a subject in need thereof.

Aspect 14 In another aspect, the disclosure provides a method of aspect 13 wherein the cell expresses PTN and RPTP β/ζ.

Aspect 15 In another aspect, the disclosure provides a method of aspect 13 or 14 wherein when an effective amount of antibody against PTN or fragment thereof is administered to the subject, the antibody substantially binds to PTN, and wherein RPTP β/ζ is consequently no longer substantially inactivated, and wherein ALK is consequently substantially dephosphorylated by RPTP β/ζ, and wherein dephosphorylated ALK fails to substantially phosphorylate tyrosine-333, and wherein RPTP β/ζ consequently substantially dephosphorylates tyrosine-333.

Aspect 16 In another aspect, the disclosure provides a method for reducing the phosphorylation state of tyrosine-333 of β-catenin in a cell of a subject comprising administering a medicament including an effective amount of an antibody against PTN or a fragment thereof, to a subject in need thereof.

Aspect 17 In another aspect, the disclosure provides a method of aspect 16 wherein the cell expresses PTN and RPTP β/ζ.

Aspect 18 In another aspect, the disclosure provides a method of aspect 16 or 17 wherein when an effective amount of antibody against PTN or fragment thereof is administered to the subject, the antibody substantially binds to PTN, and wherein RPTP β/ζ is consequently no longer substantially inactivated, and wherein ALK is consequently substantially dephosphorylated by RPTP β/ζ, and wherein dephosphorylated ALK fails to substantially phosphorylate tyrosine-333, reducing the phosphorylation state of the tyrosine-333 of β-catenin.

Aspect 19. In another aspect, the disclosure provides a method for preventing disruption of cell-cell adhesion in a cell of a subject comprising administering a medicament including an effective amount of an antibody against PTN or a fragment thereof, to a subject in need thereof.

Aspect 20. In another aspect, the disclosure provides a method of aspect 19, wherein the cell expresses PTN and RPTP β/ζ.

Aspect 21. In another aspect, the disclosure provides a method of aspect 19 or 20, wherein E-cadherin is expressed at a higher level than any other cadherins in the cell.

Aspect 22. In another aspect, the disclosure provides a method for inhibiting the reduction of cell-cell adhesion in a cell of a subject comprising administering an effective amount of a medicament including an antibody against PTN or a fragment thereof, to a subject in need thereof.

Aspect 23. In another aspect, the disclosure provides a method of aspect 22, wherein the cell expresses PTN and RPTP β/ζ.

Aspect 24. In another aspect, the disclosure provides a method of aspect 22 or 23, wherein E-cadherin and N-cadherin are expressed at a higher level than any other cadherins in the cell.

Aspect 25. In another aspect, the disclosure provides a method for reducing the disruption of a bond between β-catenin and E-cadherin in a cell of a subject comprising administering an effective amount of an antibody against PTN or a fragment thereof, to a subject in need thereof.

Aspect 26. In another aspect, the disclosure provides a method of aspect 25, wherein the cell expresses PTN and RPTP β/ζ.

Aspect 27. In another aspect, the disclosure provides a method of aspect 25 or 26 wherein E-cadherin and N-cadherin are expressed at a higher level than any other cadherins in the cell.

Aspect 28. In another aspect, the disclosure provides a method for reducing E-cadherin degradation by a cell's ubiquitin pathway in a cell of a subject comprising administering an effective amount of an antibody against PTN or a fragment thereof, to a subject in need thereof.

Aspect 29. In another aspect, the disclosure provides a method of aspect 28, wherein the cell expresses PTN and RPTP β/ζ.

Aspect 30. In another aspect, the disclosure provides a method of aspect 28 or 29, wherein E-cadherin is expressed at a higher level than any other cadherins in the cell.

Aspect 31. In another aspect, the disclosure provides a method for inhibiting upregulation of E-cadherin or N-cadherin in a cell of a subject comprising administering a medicament including an effective amount of an antibody against PTN or a fragment thereof, to a subject in need thereof.

Aspect 32. In another aspect, the disclosure provides a method of aspect 31, wherein the cell expresses PTN and RPTP β/ζ.

Aspect 33. In another aspect, the disclosure provides a method for decreasing expression of E-cadherin or N-cadherin in a cell of a subject comprising administering an effective amount of an antibody against PTN or a fragment thereof, to a subject in need thereof.

Aspect 34. In another aspect, the disclosure provides a method of aspect 33, wherein the cell expresses PTN and RPTP β/ζ.

Aspect 35. In another aspect, the disclosure provides a method for decreasing the phosphorylation of tyrosine-333 of β-catenin in a cell of a subject comprising administering an effective amount of an antibody against PTN or a fragment thereof, to a subject in need thereof.

Aspect 36. In another aspect, the disclosure provides a method of aspect 35, wherein the cell expresses PTN and RPTP β/ζ.

Aspect 37. In another aspect, the disclosure provides a method for decreasing expression or levels of β-catenin in a cell of a subject comprising administering an effective amount of an antibody against PTN or a fragment thereof, to a subject in need thereof.

Aspect 38. In another aspect, the disclosure provides a method of aspect 37, wherein the cell expresses PTN and RPTP β/ζ.

Aspect 39. In another aspect, the disclosure provides a method for inhibiting upregulation of at least one of integrin α1, α2, α4, and α5 in a cell of a subject comprising administering an effective amount of an antibody against PTN or a fragment thereof, to a subject in need thereof.

Aspect 40. In another aspect, the disclosure provides a method for decreasing expression of at least one of integrin α1, α2, α4, and α5 in a cell of a subject comprising administering an effective amount of an antibody against PTN or a fragment thereof, to a subject in need thereof.

Aspect 41. In another aspect, the disclosure provides a method of aspect 40, wherein the cell expresses PTN and RPTP β/ζ.

Aspect 42. In another aspect, the disclosure provides a method for inhibiting downregulation of integrin α3 in a cell of a subject comprising administering an effective amount of an antibody against PTN or a fragment thereof, to a subject in need thereof.

Aspect 43. In another aspect, the disclosure provides a method of aspect 42, wherein the cell expresses PTN and RPTP β/ζ.

Aspect 44. In another aspect, the disclosure provides a method for increasing expression of integrin α3 in a cell of a subject comprising administering an effective amount of an antibody against PTN or a fragment thereof, to a subject in need thereof.

Aspect 45. In another aspect, the disclosure provides a method of aspect, 44 wherein the cell expresses PTN and RPTP β/ζ.

Aspect 46. In another aspect, the disclosure provides a method for inhibiting downregulation of keratin 20 in a cell of a subject comprising administering an effective amount of an antibody against PTN or a fragment thereof, to a subject in need thereof.

Aspect 47. In another aspect, the disclosure provides a method for increasing expression of keratin 20 in a cell of a subject comprising administering an effective amount of an antibody against PTN or a fragment thereof, to a subject in need thereof.

Aspect 48. In another aspect, the disclosure provides a method of aspect 47, wherein the cell expresses PTN and RPTP β/ζ.

Aspect 49. In another aspect, the disclosure provides a method for inhibiting upregulation of keratin 10 in a cell of a subject comprising administering an effective amount of an antibody against PTN or a fragment thereof, to a subject in need thereof.

Aspect 50. In another aspect, the disclosure provides a method of aspect 49, wherein the cell expresses PTN and RPTP β/ζ.

Aspect 51. In another aspect, the disclosure provides a method for decreasing expression of keratin 10 in a cell of a subject comprising administering an effective amount of an antibody against PTN or a fragment thereof, to a subject in need thereof.

Aspect 52. In another aspect, the disclosure provides a method of aspect 51, wherein the cell expresses PTN and RPTP β/ζ.

Aspect 53. In another aspect, the disclosure provides a method for reducing concentrations of β-catenin in the nucleus in a cell of a subject comprising administering an effective amount of an antibody against PTN or a fragment thereof, to a subject in need thereof.

Aspect 54. In another aspect, the disclosure provides a method of aspect 53, wherein the cell expresses PTN and RPTP β/ζ.

Aspect 55. In another aspect, the disclosure provides a method for reducing import of β-catenin into the nucleus in a cell of a subject comprising administering an effective amount of an antibody against PTN or a fragment thereof, to a subject in need thereof.

Aspect 56. In another aspect, the disclosure provides a method of aspect 55, wherein the cell expresses PTN and RPTP β/ζ.

Aspect 57. In another aspect, the disclosure provides a method for reducing activation of epidermal growth factor receptor (EGFR) in a cell of a subject comprising administering a medicament including an effective amount of an antibody against PTN or a fragment thereof, to a subject in need thereof.

Aspect 58. In another aspect, the disclosure provides a method of aspect 57, wherein the cell expresses PTN and RPTP β/ζ.

Aspect 59. In another aspect, the disclosure provides a method for increasing ubiquitination of β-catenin in a cell of a subject comprising administering an effective amount of an antibody against PTN or a fragment thereof, to a subject in need thereof.

Aspect 60. In another aspect, the disclosure provides a method of aspect 59, wherein the cell expresses PTN and RPTP β/ζ.

Aspect 61. In another aspect, the disclosure provides a method for modulating, for example reducing phosphorylation of EGFR in a cell of a subject comprising administering an effective amount of an antibody against PTN or a fragment thereof, to a subject in need thereof.

Aspect 62. In another aspect, the disclosure provides a method of aspect 61, wherein the cell expresses PTN and RPTP β/ζ.

Aspect 63. In another aspect, the disclosure provides a method for reducing the transcription of genes induced by Tcf/Lef family in a cell of a subject comprising administering an effective amount of an antibody against PTN or a fragment thereof, to a subject in need thereof.

Aspect 64. In another aspect, the disclosure provides a method of aspect 63, wherein the cell expresses PTN and RPTP β/ζ.

Aspect 65. In another aspect, the disclosure provides a method for reducing activation of Akt in a cell of a subject comprising administering an effective amount of an antibody against PTN or a fragment thereof, to a subject in need thereof.

Aspect 66. In another aspect, the disclosure provides a method of aspect 65, wherein the cell expresses PTN and RPTP β/ζ.

Aspect 67. In another aspect, the disclosure provides a method for reducing phosphorylation of Akt in a cell of a subject comprising administering an effective amount of an antibody against PTN or a fragment thereof, to a subject in need thereof.

Aspect 68. In another aspect, the disclosure provides a method of aspect 67 wherein, the cell expresses PTN and RPTP β/ζ.

Aspect 69. In another aspect, the disclosure provides a method for increasing activation of GSK3β (Glycogen Synthase Kinase 3β) in a cell of a subject comprising administering an effective amount of an antibody against PTN or a fragment thereof, to a subject in need thereof.

Aspect 70. In another aspect, the disclosure provides a method of aspect 69, wherein the cell expresses PTN and RPTP β/ζ.

Aspect 71. In another aspect, the disclosure provides a method for decreasing phosphorylation of GSK3β in a cell of a subject comprising administering an effective amount of an antibody against PTN or a fragment thereof, to a subject in need thereof.

Aspect 72. In another aspect, the disclosure provides a method of aspect 71, wherein the cell expresses PTN and RPTP β/ζ.

Aspect 73. In another aspect, the disclosure provides a method for reducing the concentration of γ-catenin in the nucleus in a cell of a subject comprising administering an effective amount of an antibody against PTN or a fragment thereof, to a subject in need thereof.

Aspect 74. In another aspect, the disclosure provides a method of aspect 73, wherein the cell expresses PTN and RPTP β/ζ.

Aspect 75. In another aspect, the disclosure provides a method for downregulating expression and/or decreasing levels of one or more downstream targets of the PTN/RPTPβ/ζ signaling pathway including at least MDR1, ZO1, uPAR, c-jun, survivin, DRCTNNB1A, PPAR ∝, Id2, TCF-1, Brachyury, NBL4, c-myc, and ITF-2 in a cell of a subject comprising administering an effective amount of an antibody against PTN or a fragment thereof, to a subject in need thereof.

Aspect 76. In another aspect, the disclosure provides a method of aspect 75, wherein the cell expresses PTN and RPTP β/ζ.

Aspect 77. In another aspect, the disclosure provides a method for upregulating expression and/or increasing levels of one or more of Cyclin D1, Fra-1 and Connexin-43 in a cell of a subject comprising administering an effective amount of an antibody against PTN or a fragment thereof, to a subject in need thereof.

Aspect 78. In another aspect, the disclosure provides a method of aspect 77, wherein the cell expresses PTN and RPTP β/ζ.

Aspect 79. In another aspect, the disclosure provides a method for reducing serine phosphorylation of β-adducin (adducin 2β) in a cell of a subject comprising administering an effective amount of an antibody against PTN or a fragment thereof, to a subject in need thereof.

Aspect 80. In another aspect, the disclosure provides a method of aspect 79, wherein the cell expresses PTN and RPTP β/ζ.

Aspect 81. In another aspect, the disclosure provides a method for reducing the phosphorylation of GIT1/Cat-1 in a cell of a subject comprising administering an effective amount of an antibody against PTN or a fragment thereof, to a subject in need thereof.

Aspect 82. In another aspect, the disclosure provides a method of aspect 81, the cell expresses PTN and RPTP β/ζ.

Aspect 83. In another aspect, the disclosure provides a method for reducing the phosphorylation of P190RhoGAP in a cell of a subject comprising administering an effective amount of an antibody against PTN or a fragment thereof, to a subject in need thereof.

Aspect 84. In another aspect, the disclosure provides a method of aspect 83, wherein the cell expresses PTN and RPTP β/ζ.

Aspect 85. In another aspect, the disclosure provides a method for reducing phosphorylation of HDAC-2 in a cell of a subject comprising administering an effective amount of an antibody against PTN or a fragment thereof, to a subject in need thereof.

Aspect 86. In another aspect, the disclosure provides a method of aspect 85, wherein the cell expresses PTN and RPTP β/ζ.

Aspect 87. In another aspect, the disclosure provides a method for reducing phosphorylation of FYN in a cell of a subject comprising administering an effective amount of an antibody against PTN or a fragment thereof, to a subject in need thereof.

Aspect 88. In another aspect, the disclosure provides a method of aspect 87, wherein the cell expresses PTN and RPTP β/ζ.

Aspect 89. In another aspect, the disclosure provides a method for decreasing steady-state phosphorylation levels of molecular targets of RPTP β/ζ in a cell of a subject comprising administering an effective amount of an antibody against PTN or a fragment thereof, to a subject in need thereof.

Aspect 90. In another aspect, the disclosure provides a method of aspect 89, wherein the cell expresses PTN and RPTP β/ζ.

Aspect 91. In another aspect, the disclosure provides a method for decreasing steady-state phosphorylation levels of molecular targets of ALK in a cell of a subject comprising administering an effective amount of an antibody against PTN or a fragment thereof, to a subject in need thereof.

Aspect 92. In another aspect, the disclosure provides a method of aspect 91, wherein the cell expresses PTN and RPTP β/ζ.

Aspect 93. In another aspect, the disclosure provides a method for preventing a cell of a subject from progressing to a malignant cancer cell comprising administering an effective amount a medicament including of an antibody against PTN or a fragment thereof, to a subject in need thereof.

Aspect 94. In another aspect, the disclosure provides a method of aspect 93, wherein the cell expresses PTN and RPTP β/ζ.

Aspect 95. In another aspect, the disclosure provides a method for reducing deposition of collagen by a tumor cell comprising administering an effective amount of a medicament including an antibody against PTN or a fragment thereof, to a subject in need thereof

Aspect 96. In another aspect, the disclosure provides a method of aspect 95, wherein the cell expresses PTN and RPTP β/ζ.

Aspect 97. In another aspect, the disclosure provides a method for reducing deposition of elastin by a tumor cell comprising administering an effective amount of a medicament including antibody against PTN or a fragment thereof, to a subject in need thereof

Aspect 98. In another aspect, the disclosure provides a method of aspect 97, wherein the cell expresses PTN and RPTP β/ζ.

Aspect 99. In another aspect, the disclosure provides a method for reducing secretion of collagen by a tumor cell in a subject comprising administering an effective amount a medicament including of an antibody against PTN or a fragment thereof, to a subject in need thereof

Aspect 100. In another aspect, the disclosure provides a method of aspect 99, wherein the cell expresses PTN and RPTP β/ζ.

Aspect 101. In another aspect, the disclosure provides a method for reducing secretion of elastin by a tumor cell in a subject comprising administering an effective amount of a medicament including an antibody against PTN or a fragment thereof, to a subject in need thereof

Aspect 102. In another aspect, the disclosure provides a method of aspect 101, wherein the cell expresses PTN and RPTP β/ζ.

Aspect 103. In another aspect, the disclosure provides a method for reducing the incidence of cancer in a subject comprising administering a medicament including an effective amount of an antibody against PTN or a fragment thereof, to a subject in need thereof.

Aspect 104. In another aspect, the disclosure provides a method of aspect 103, wherein the cell expresses PTN and RPTP β/ζ.

Aspect 105. In another aspect, the disclosure provides a method for reducing the malignancy of a cancer in a subject comprising administering an effective amount of a medicament including an antibody against PTN or a fragment thereof, to a subject in need thereof.

Aspect 106. The method of aspect 105 wherein the cancer is comprised of one or more cells that express PTN, RPTP β/ζ and ALK.

Aspect 107. In another aspect, the disclosure provides a method for reducing scirrhous patterned carcinoma type breast cancer in a subject comprising administering an effective amount of a medicament including an antibody against PTN or a fragment thereof, to a subject in need thereof.

Aspect 108. In another aspect, the disclosure provides a method of aspect 107, wherein the scirrhous patterned carcinoma type breast cancer is comprised of one or more cells that express PTN, RPTP β/ζ and ALK.

Aspect 109. In another aspect, the disclosure provides a method for reversing EMT in a cell of a subject comprising administering an effective amount of a medicament including an antibody against PTN or a fragment thereof, to a subject in need thereof.

Aspect 110. In another aspect, the disclosure provides a method of aspect 109, wherein the cell expresses PTN and RPTP β/ζ.

Aspect 111. In another aspect, the disclosure provides a method for preventing a cell from undergoing an EMT in a subject comprising administering an effective amount of a medicament including an antibody against PTN or a fragment thereof, to a subject in need thereof.

Aspect 112. In another aspect, the disclosure provides a method of aspect 111, wherein the cell expresses PTN and RPTP β/ζ.

Aspect 113. In another aspect, the disclosure provides a method for reducing activation of Insulin-like Growth Factor 1 Receptor (IGF-1 Receptor; IGFR-1) in a cell of a subject comprising administering an effective amount of an antibody against PTN or a fragment thereof, to a subject in need thereof.

Aspect 114. In another aspect, the disclosure provides a method of aspect 113, wherein the cell expresses PTN and RPTP β/ζ.

Aspect 115. In another aspect, the disclosure provides a method for reducing phosphorylation of Insulin-like Growth Factor 1 Receptor (IGF-1 Receptor; IGFR-1) in a cell of a subject comprising administering an effective amount of an antibody against PTN or a fragment thereof, to a subject in need thereof.

Aspect 116. In another aspect, the disclosure provides a method of aspect 115, wherein the cell expresses PTN and RPTP β/ζ.

Aspect 117. In another aspect, the disclosure provides a method for reducing activation of Human Epidermal Growth Factor Receptor 2 (HER2/neu, also known as ErbB-2) in a cell of a subject comprising administering an effective amount of an antibody against PTN or a fragment thereof, to a subject in need thereof.

Aspect 118. In another aspect, the disclosure provides a method of aspect 117, wherein the cell expresses PTN and RPTP β/ζ.

Aspect 119. In another aspect, the disclosure provides a method for reducing phosphorylation of Human Epidermal Growth Factor Receptor 2 (HER2/neu, also known as ErbB-2) in a cell of a subject comprising administering an effective amount of an antibody against PTN or a fragment thereof, to a subject in need thereof.

Aspect 120. In another aspect, the disclosure provides a method of aspect 119, wherein the cell expresses PTN and RPTP β/ζ.

Aspect 121. In another aspect, the disclosure provides a method for reducing activation of Vascular Endothelial Growth Factor Receptor 1 (VEGFR-1, Flt-1) in a cell of a subject comprising administering an effective amount of an antibody against PTN or a fragment thereof, to a subject in need thereof.

Aspect 122. In another aspect, the disclosure provides a method of aspect 121, wherein the cell expresses PTN and RPTP β/ζ.

Aspect 123. In another aspect, the disclosure provides a method for reducing phosphorylation of Vascular Endothelial Growth Factor Receptor 1 (VEGFR-1, Flt-1) in a cell of a subject comprising administering an effective amount of an antibody against PTN or a fragment thereof, to a subject in need thereof.

Aspect 124. In another aspect, the disclosure provides a method of aspect 123, wherein the cell expresses PTN and RPTP β/ζ.

Aspect 125. In another aspect, the disclosure provides a method for reducing activation of Vascular Endothelial Growth Factor Receptor 2 (VEGFR-2, KDR/Flk-1) in a cell of a subject comprising administering an effective amount of an antibody against PTN or a fragment thereof, to a subject in need thereof.

Aspect 126. In another aspect, the disclosure provides a method of aspect 125, wherein the cell expresses PTN and RPTP β/ζ.

Aspect 127. In another aspect, the disclosure provides a method for reducing phosphorylation of Vascular Endothelial Growth Factor Receptor 2 (VEGFR-2, KDR/Flk-1) in a cell of a subject comprising administering an effective amount of an antibody against PTN or a fragment thereof, to a subject in need thereof.

Aspect 128. In another aspect, the disclosure provides a method of aspect 127, wherein the cell expresses PTN and RPTP β/ζ.

Aspect 129. In another aspect, the disclosure provides a method for reducing activation of VEGFR-3 in a cell of a subject comprising administering an effective amount of an antibody against PTN or a fragment thereof, to a subject in need thereof.

Aspect 130. In another aspect, the disclosure provides a method of aspect 129, wherein the cell expresses PTN and RPTP β/ζ.

Aspect 131. In another aspect, the disclosure provides a method for reducing phosphorylation of VEGFR-3 in a cell of a subject comprising administering an effective amount of an antibody against PTN or a fragment thereof, to a subject in need thereof.

Aspect 132. In another aspect, the disclosure provides a method of aspect 131, wherein the cell expresses PTN and RPTP β/ζ.

Aspect 133. In another aspect, the disclosure provides a method for substantially deactivating Receptor Tyrosine-Protein Kinase erbB-4 (erbB-4) in a cell of a subject comprising administering an effective amount of an antibody against PTN or a fragment thereof, to a subject in need thereof.

Aspect 134. In another aspect, the disclosure provides a method for reducing the activity of Receptor Tyrosine-Protein Kinase erbB-4 (erbB-4) in a cell of a subject comprising administering an effective amount of an antibody against PTN or a fragment thereof, to a subject in need thereof.

Aspect 135. In another aspect, the disclosure provides a method for reducing the phosphorylation state of Receptor Tyrosine-Protein Kinase erbB-4 (erbB-4) in a cell of a subject comprising administering an effective amount of an antibody against PTN or a fragment thereof, to a subject in need thereof.

Aspect 136. In another aspect, the disclosure provides a method for substantially deactivating Protein Kinase C (PKC) in a cell of a subject comprising administering an effective amount of an antibody against PTN or a fragment thereof, to a subject in need thereof.

Aspect 137. In another aspect, the disclosure provides a method for reducing the activity of Protein Kinase C (PKC) in a cell of a subject comprising administering an effective amount of an antibody against PTN or a fragment thereof, to a subject in need thereof.

Aspect 138. In another aspect, the disclosure provides a method for reducing the phosphorylation state of Protein Kinase C (PKC) in a cell of a subject comprising administering an effective amount of an antibody against PTN or a fragment thereof, to a subject in need thereof.

Aspect 139. In another aspect, the disclosure provides a method for substantially deactivating Leukocyte Receptor Tyrosine Kinase (LTK) in a cell of a subject comprising administering an effective amount of an antibody against PTN or a fragment thereof, to a subject in need thereof.

Aspect 140. In another aspect, the disclosure provides a method for reducing the activity of Leukocyte Receptor Tyrosine Kinase (LTK) in a cell of a subject comprising administering an effective amount of an antibody against PTN or a fragment thereof, to a subject in need thereof.

Aspect 141. In another aspect, the disclosure provides a method for reducing the phosphorylation state of Leukocyte Receptor Tyrosine Kinase (LTK) in a cell of a subject comprising administering an effective amount of an antibody against PTN or a fragment thereof, to a subject in need thereof.

Aspect 142. In another aspect, the disclosure provides a method of any of aspects 1-141, wherein the cancer, tumor or cell is from a cancer selected from the group consisting of adrenocortical carcinoma, AIDS-related cancers, AIDS-related lymphoma, anal cancer, appendix cancer, astrocytoma (childhood), cerebellar or cerebral, basal cell carcinoma, bile duct cancer, extrahepatic (bile duct) cancer, bladder cancer, bone cancer, osteosarcoma/malignant fibrous histiocytoma, brainstem glioma, cerebellar astrocytoma, cerebral astrocytoma/malignant glioma, ependymoma, medulloblastoma, supratentorial primitive neuroectodermal tumors, visual pathway and hypothalamic glioma, breast cancer (e.g., DCIS (Ductal Carcinoma In Situ), LCIS (Lobular Carcinoma In Situ), IDC (Invasive Ductal Carcinoma), e.g., scirrhous carcinoma, less common subtypes of Invasive Ductal Carcinoma (e.g., tubular carcinoma of the breast, medullary carcinoma of the breast, mucinous carcinoma of the breast, papillary carcinoma of the breast, and cribriform carcinoma of the breast), ILC (Invasive Lobular Carcinoma), Paget's Disease of the Nipple, Inflammatory Breast Cancer, Male Breast Cancer, Recurrent and Metastatic Breast Cancer, and phyllodes tumors, e.g., cystosarcoma phyllodes), bronchial adenomas/carcinoids, Burkitt lymphoma, carcinoid tumor (childhood), carcinoid tumor, gastrointestinal, carcinoma of unknown primary, central nervous system lymphoma (primary), cerebellar astrocytoma (childhood), cerebral astrocytoma/Malignant glioma (childhood), cervical cancer, childhood cancers, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor, extragonadal Germ cell tumor, extrahepatic bile duct cancer, eye cancer, intraocular melanoma, eye cancer, retinoblastoma, gallbladder cancer, gastric (stomach) cancer, gastrointestinal carcinoid tumor, gastrointestinal stromal tumor (GIST), germ cell tumor (extracranial), germ cell tumor (extragonadal), germ cell tumor (ovarian), gestational trophoblastic tumor, glioma (adult), glioma (childhood), childhood cerebral astrocytoma, glioma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma (childhood), intraocular melanoma, islet cell carcinoma (endocrine pancreas), Kaposi sarcoma, laryngeal cancer, hairy cell, lip and oral cavity cancer, liver cancer (primary), lung cancer (non-small cell), lung cancer (small cell), lymphomas, lymphoma (AIDS-related), lymphoma (Burkitt), lymphoma (cutaneous T-cell), lymphoma (Hodgkin), lymphomas (non-Hodgkin), lymphoma (primary central nervous system), macroglobulinemia, Waldenström, malignant fibrous histiocytoma of bone/osteosarcoma, medulloblastoma (childhood), melanoma, melanoma (intraocular (eye)), merkel cell carcinoma, mesothelioma (adult), mesothelioma (childhood), metastatic squamous neck cancer with occult primary, mouth cancer, multiple endocrine neoplasia syndrome (childhood), mycosis fungoides, myelodysplastic syndromes, myelodysplastic/myeloproliferative diseases, myelogenous leukemia (chronic), myeloid leukemia (adult acute), myeloid leukemia (childhood acute), myeloma (multiple (cancer of the bone-marrow)), myeloproliferative disorders (chronic), nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma (childhood), salivary gland cancer, sarcoma (soft tissue), sarcoma (uterine), Sézary syndrome, skin cancer (nonmelanoma), skin cancer (melanoma), skin carcinoma, Merkel cell, small intestine cancer, soft tissue sarcoma, squamous cell carcinoma, squamous neck cancer with occult primary, stomach cancer, supratentorial primitive neuroectodermal tumor (childhood), T-cell lymphoma, cutaneous (e.g., mycosis fungoides and Sézary syndrome), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site carcinoma, ureter and renal pelvis, transitional cell cancer, urethral cancer, uterine cancer, endometrial, uterine sarcoma, vaginal cancer, visual pathway and hypothalamic glioma (childhood), vulvar cancer, Waldenström macroglobulinemia, and Wilms tumor (kidney cancer).

Aspect 143. In another aspect, the disclosure provides a method of aspect 142, wherein the cancer is breast cancer.

Aspect 144. In another aspect, the disclosure provides a method of aspect 143, wherein the breast cancer is selected from the group consisting of DCIS (Ductal Carcinoma In Situ), LCIS (Lobular Carcinoma In Situ), IDC (Invasive Ductal Carcinoma), tubular carcinoma of the breast, medullary carcinoma of the breast, mucinous carcinoma of the breast, papillary carcinoma of the breast, cribriform carcinoma of the breast, ILC (Invasive Lobular Carcinoma), Paget's Disease of the Nipple, Inflammatory Breast Cancer, Male Breast Cancer, Recurrent and Metastatic Breast Cancer, and cystosarcoma phyllodes.

Aspect 145. In another aspect, the disclosure provides a method of aspect 144, wherein the breast cancer is DCIS.

Aspect 146. In another aspect, the disclosure provides a method of aspect 144, wherein the breast cancer is LCIS.

Aspect 147. In another aspect, the disclosure provides a method of aspect 144, wherein the breast cancer is IDC.

Aspect 148. In another aspect, the disclosure provides a method of aspect 147, wherein the IDC is scirrhous carcinoma.

Aspect 149. In another aspect, the disclosure provides a method of aspect 144 wherein the breast cancer is tubular carcinoma of the breast.

Aspect 150. In another aspect, the disclosure provides a method of aspect 144, wherein the breast cancer is medullary carcinoma of the breast.

Aspect 151. In another aspect, the disclosure provides a method of aspect 144, wherein the breast cancer is mucinous carcinoma of the breast.

Aspect 152. In another aspect, the disclosure provides a method of aspect 144, wherein the breast cancer is papillary carcinoma of the breast.

Aspect 153. In another aspect, the disclosure provides a method of aspect 144, wherein the breast cancer is cribriform carcinoma of the breast.

Aspect 154. In another aspect, the disclosure provides a method of aspect 144, wherein the breast cancer is ILC (Invasive Lobular Carcinoma).

Aspect 155. In another aspect, the disclosure provides a method of aspect 144, wherein the breast cancer is Paget's Disease of the Nipple.

Aspect 156. In another aspect, the disclosure provides a method of aspect 144, wherein the breast cancer is Inflammatory Breast Cancer.

Aspect 157. In another aspect, the disclosure provides a method of aspect 144, wherein the breast cancer is Male Breast Cancer.

Aspect 158. In another aspect, the disclosure provides a method of aspect 144, wherein the breast cancer is Recurrent and Metastatic Breast Cancer.

Aspect 159. In another aspect, the disclosure provides a method of aspect 144, wherein the breast cancer is cystosarcoma phyllodes.

Aspect 160. In another aspect, the disclosure provides a method according to any of aspects 1-159, wherein the cancer, tumor or cell is comprised of at least one cell that expresses PTN and RPTP β/ζ.

Aspect 161. In another aspect, the disclosure provides a method of any of aspects 1-159, wherein the cancer, tumor or cell is comprised of at least one cell that expresses PTN, RPTP β/ζ and ALK.

Aspect 162. In another aspect, the disclosure provides a method according to any one of aspects 1-6 or 11-159, wherein the antibody is a monoclonal antibody.

Aspect 163. In another aspect, the disclosure provides a method according to any one of aspects 1-6 or 11-159, wherein the antibody is a polyclonal antibody.

Aspect 164. In another aspect, the disclosure provides a method according to aspect 162 or 163, wherein the antibody is a humanized antibody.

Aspect 165. In another aspect, the disclosure provides a method according to any one of aspects 1-6 or 11-159, wherein the method comprises administering an effective amount of decoy RPTP β/ζ rather than administering an effective amount of an antibody against PTN or a fragment thereof.

Aspect 166. In another aspect, the disclosure provides a method according to any one of aspects 1-6 or 11-159, wherein the method comprises administering an effective amount of negative PTN rather than administering an effective amount of an antibody against PTN or a fragment thereof.

Aspect 167. In another aspect, the disclosure provides a method according to any one of aspects 1-6 or 11-159, wherein the method comprises administering an effective amount of one or more of an antibody against PTN or a fragment thereof, negative PTN, and decoy RPTP β/ζ, or combinations thereof.

Aspect 168. In another aspect, the disclosure provides a method according to aspect 165, 166 or 167, wherein when an effective amount of an antibody against PTN or a fragment thereof, is administered in combination with one or more of the negative PTN, and decoy RPTP β/ζ, the antibody against PTN is a monoclonal antibody or fragment thereof.

Aspect 169. In another aspect, the disclosure provides a method according to aspect 165, 166 or 167, wherein when an effective amount of an antibody against PTN or a fragment thereof, is administered in combination with one or more of the negative PTN, and decoy RPTP β/ζ, the antibody against PTN is a polyclonal antibody or fragment thereof.

Aspect 170. In another aspect, the disclosure provides a method according to aspect 168, wherein when an effective amount of an antibody against PTN or a fragment thereof, is administered in combination with one or more of the negative PTN, decoy RPTP β/ζ, the antibody against PTN is a humanized antibody or fragment thereof.

Aspect 171. In another aspect, the disclosure provides a method of aspect 168, 169 or 170, wherein when an effective amount of antibody against PTN is administered to the subject, the antibody substantially binds to PTN and RPTP β/ζ is consequently no longer substantially inactivated, and wherein when an effective amount of negative PTN is administered to the subject, the negative PTN monomers substantially bind to endogenous PTN monomers to form substantially inactive heterodimers and RPTP β/ζ is consequently no longer substantially inactivated, and wherein when an effective amount of decoy RPTP β/ζ is administered to the subject, one or more dimers of endogenous PTN binds to the decoy RPTP β/ζ and RPTP β/ζ is consequently no longer substantially inactivated.

Aspect 172. In another aspect, the disclosure provides a method of aspect 168, 169 or 170, wherein when an effective amount of antibody against PTN is administered to the subject, the antibody substantially binds to PTN, RPTP β/ζ is consequently no longer substantially inactivated and ALK is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and ALK is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and ALK is consequently substantially dephosphorylated by RPTP β/ζ.

Aspect 173. In another aspect, the disclosure provides a method of aspect 160, wherein the cancer, tumor or cell is comprised of at least one cell that express PTN, RPTP β/ζ and one or more of EGFR, IGFR-1, erbB2, erbB4, PKC, LTK and VEGFR.

Aspect 174. In another aspect, the disclosure provides a method of any of the prior aspects, wherein the cancer, tumor or cell expresses PTN, RPTP β/ζ, ALK and one or more of EGFR, IGFR-1, erbB2, erbB4, PKC, LTK and VEGFR.

Aspect 175. In another aspect, the disclosure provides a method according to any one of aspect 173 or 174, wherein the antibody is a monoclonal antibody.

Aspect 176. In another aspect, the disclosure provides a method according to any one of aspect 173 or 174, wherein the antibody is a polyclonal antibody.

Aspect 177. In another aspect, the disclosure provides a method according to aspect 175 or 176. Wherein the antibody is a humanized antibody.

Aspect 178. In another aspect, the disclosure provides a method according to any one of aspect 173 or 174, wherein the method comprises administering an effective amount of decoy RPTP β/ζ rather than administering an effective amount of an antibody against PTN or a fragment thereof.

Aspect 179. In another aspect, the disclosure provides a method according to any one of aspects 173-174, wherein the method comprises administering an effective amount of negative PTN rather than administering an effective amount of an antibody against PTN or a fragment thereof.

Aspect 180. In another aspect, the disclosure provides a method according to any one of aspect 173 or 174 wherein the method comprises administering an effective amount of one or more of an antibody against PTN or a fragment thereof, negative PTN, and decoy RPTP β/ζ, or combinations thereof.

Aspect 181. In another aspect, the disclosure provides a method according to aspect 178, 179 or 180 wherein when an effective amount of an antibody against PTN or a fragment thereof, is administered in combination with one or more of the negative PTN, and decoy RPTP β/ζ, the antibody against PTN is a monoclonal antibody or fragment thereof.

Aspect 182. In another aspect, the disclosure provides a method according to aspect 178, 179 or 180 wherein when an effective amount of an antibody against PTN or a fragment thereof, is administered in combination with one or more of the negative PTN, and decoy RPTP β/ζ, the antibody against PTN is a polyclonal antibody or fragment thereof.

Aspect 183. In another aspect, the disclosure provides a method according to aspect 181 or 182 wherein when an effective amount of an antibody against PTN or a fragment thereof, is administered in combination with one or more of the negative PTN, decoy RPTP β/ζ, and the antibody against PTN is a humanized antibody or fragment thereof.

Aspect 184. In another aspect, the disclosure provides a method of any of aspect 173 or 174, wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

Aspect 185. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

Aspect 186. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

Aspect 187. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

Aspect 188. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Receptor Tyrosine-Protein Kinase erbB-4 (erbB4) is consequently substantially dephosphorylated by RPTP β/ζ.

Aspect 189. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Protein Kinase C (PKC) is consequently substantially dephosphorylated by RPTP β/ζ.

Aspect 190. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Leukocyte Tyrosine Kinase (LTK) is consequently substantially dephosphorylated by RPTP β/ζ.

Aspect 191. In another aspect, the disclosure provides a method of aspect 184, wherein when one or more of an effective amount of antibody against PTN is administered to the subject, an effective amount of negative PTN is administered to the subject, and an effective amount of decoy RPTP β/ζ is administered to the subject, Vascular Endothelial Growth Factor Receptor (VEGFR) is consequently substantially dephosphorylated by RPTP β/ζ.

The disclosure also provides various aspects of the invention including the following.

In an aspect, the disclosure provides a method for treating cancer in a subject comprising administering an effective amount of a medicament including an antibody against PTN or a fragment thereof wherein the cancer is comprised of one or more cells that express PTN and RPTP β/ζ.

In another aspect, the disclosure provides a method for reducing risk of developing cancer in a subject comprising administering an effective amount of a medicament including an antibody against PTN or a fragment thereof wherein the cancer is comprised of one or more cells that express PTN and RPTP β/ζ.

In another aspect, the disclosure provides a method for diminishing the likelihood of development of a cancer in a subject comprising administering an effective amount of a medicament including an antibody against PTN or a fragment thereof wherein the cancer is comprised of one or more cells that express PTN and RPTP β/ζ.

In another aspect, the disclosure provides a method for reversing tumor growth in a subject comprising administering an effective amount of a medicament including an antibody against PTN or a fragment thereof wherein the tumor is comprised of one or more cells that express PTN and RPTP β/ζ.

In another aspect, the disclosure provides a method for inhibiting tumor growth in a subject comprising administering an effective amount of a medicament including an antibody against PTN or a fragment thereof wherein the tumor is comprised of one or more cells that express PTN and RPTP β/ζ.

In another aspect, the disclosure provides a method for reducing cancer cell proliferation in a subject comprising administering an effective amount of a medicament including an antibody against PTN or a fragment thereof wherein the cancer cell expresses PTN and RPTP β/ζ.

In another aspect, the disclosure provides a method for reducing cancer cell invasiveness comprising administering an effective amount of a medicament including an antibody against PTN or a fragment thereof wherein the cancer cell expresses PTN and RPTP β/ζ.

In another aspect, the disclosure provides a method for reducing cancer cell motility comprising administering an effective amount of a medicament including an antibody against PTN or a fragment thereof wherein the cancer cell expresses PTN and RPTP β/ζ.

In another aspect, the disclosure provides a method for reducing cancer cell metastasis comprising administering an effective amount of a medicament including an antibody against PTN or a fragment thereof wherein the cancer cell expresses PTN and RPTP β/ζ.

In another aspect, the disclosure provides a method for increasing the effectiveness of anticancer treatment of a cancer cell in a subject comprising administering chemotherapy treatment and administering an effective amount of a medicament including an antibody against PTN or a fragment thereof to the subject wherein the cancer cell expresses PTN and RPTP β/ζ.

In another aspect, the disclosure provides a method for increasing the effectiveness of anticancer treatment of a cancer cell in a subject in a subject comprising administering radiation therapy and administering an effective amount of a medicament including an antibody against PTN or a fragment thereof to the subject wherein the cancer cell expresses PTN and RPTPβ/ζ.

In another aspect, the disclosure provides a method for reducing tumor cell angiogenesis in a subject comprising administering an effective amount of a medicament including an antibody against PTN or a fragment thereof wherein the tumor cell expresses PTN and RPTP β/ζ.

In another aspect, the disclosure provides a method for treating a cell that constitutively expresses PTN in a subject comprising administering an effective amount of a medicament including an antibody against PTN or a fragment thereof wherein the cell expresses PTN and RPTP β/ζ.

In another aspect, the disclosure provides a method for treating a cell that expresses PTN constitutively in a subject comprising administering an effective amount of a medicament including an antibody against PTN or a fragment thereof wherein the cell expresses PTN and RPTP β/ζ.

In another aspect, the disclosure provides a method for reducing the concentration of uncomplexed PTN in a cell of a subject comprising administering an effective amount of an antibody against PTN or a fragment thereof wherein the cell expresses PTN and RPTP β/ζ.

In another aspect, the disclosure provides a method for increasing the concentration uncomplexed monomeric RPTP β/ζ in a cell of a subject comprising administering an effective amount of an antibody against PTN or a fragment thereof wherein the cell expresses PTN and RPTP β/ζ.

In another aspect, the disclosure provides a method for decreasing the concentration of (PTN)2-(RPTP β/ζ)2 heterotetramer in a cell of a subject comprising administering an effective amount of an antibody against PTN or a fragment thereof wherein the cell expresses PTN and RPTP β/ζ.

In another aspect, the disclosure provides a method for substantially reducing activity of ALK in a cell of a subject comprising administering an effective amount of an antibody against PTN or a fragment thereof wherein the cell expresses PTN and RPTP β/ζ.

In another aspect, the disclosure provides a method for reducing the activity of ALK in a cell of a subject comprising administering an effective amount of an antibody against PTN or a fragment thereof wherein the cell expresses PTN and RPTP β/ζ.

In another aspect, the disclosure provides a method for reducing the phosphorylation state of ALK in a cell of a subject comprising administering an effective amount of an antibody against PTN or a fragment thereof wherein the cell expresses PTN and RPTP β/ζ.

In another aspect, the disclosure provides a method for substantially deactivating Receptor Tyrosine-Protein Kinase erbB-4 (erbB-4) in a cell of a subject comprising administering an effective amount of an antibody against PTN or a fragment thereof wherein the cell expresses PTN and RPTP β/ζ.

In another aspect, the disclosure provides a method for reducing the activity of Receptor Tyrosine-Protein Kinase erbB-4 (erbB-4) in a cell of a subject comprising administering an effective amount of an antibody against PTN or a fragment thereof wherein the cell expresses PTN and RPTP β/ζ.

In another aspect, the disclosure provides a method for reducing the phosphorylation state of Receptor Tyrosine-Protein Kinase erbB-4 (erbB-4) in a cell of a subject comprising administering an effective amount of an antibody against PTN or a fragment thereof wherein the cell expresses PTN and RPTP β/ζ.

In another aspect, the disclosure provides a method for substantially deactivating Protein Kinase C (PKC) in a cell of a subject comprising administering an effective amount of an antibody against PTN or a fragment thereof. wherein the cell expresses PTN and RPTP β/ζ.

In another aspect, the disclosure provides a method for reducing the activity of Protein Kinase C (PKC) in a cell of a subject comprising administering an effective amount of an antibody against PTN or a fragment thereof wherein the cell expresses PTN and RPTP β/ζ.

In another aspect, the disclosure provides a method for reducing the phosphorylation state of Protein Kinase C (PKC) in a cell of a subject comprising administering an effective amount of an antibody against PTN or a fragment thereof wherein the cell expresses PTN and RPTP β/ζ.

In another aspect, the disclosure provides a method for substantially deactivating Leukocyte Receptor Tyrosine Kinase (LTK) in a cell of a subject comprising administering an effective amount of an antibody against PTN or a fragment thereof wherein the cell expresses PTN and RPTP β/ζ.

In another aspect, the disclosure provides a method for reducing the activity of Leukocyte Receptor Tyrosine Kinase (LTK) in a cell of a subject comprising administering an effective amount of an antibody against PTN or a fragment thereof wherein the cell expresses PTN and RPTP β/ζ.

In another aspect, the disclosure provides a method for reducing the phosphorylation state of Leukocyte Receptor Tyrosine Kinase (LTK) in a cell of a subject comprising administering an effective amount of an antibody against PTN or a fragment thereof wherein the cell expresses PTN and RPTP β/ζ.

In another aspect, the disclosure provides a method for treating cancer in a subject comprising administering an effective amount of an antibody against PTN or a fragment thereof wherein the cancer is comprised of one or more cells that express PTN, RPTP β/ζ and ALK.

In another aspect, the disclosure provides a method for reducing risk of developing cancer in a subject comprising administering an effective amount of an antibody against PTN or a fragment thereof wherein the cancer is comprised of one or more cells that express PTN, RPTP β/ζ and ALK.

In another aspect, the disclosure provides a method for diminishing the likelihood of development of a cancer in a subject comprising administering an effective amount of a medicament including an antibody against PTN or a fragment thereof wherein the cancer is comprised of one or more cells that express PTN, RPTP β/ζ and ALK.

In another aspect, the disclosure provides a method for reversing tumor growth in a subject comprising administering an effective amount of a medicament including an antibody against PTN or a fragment thereof wherein the tumor is comprised of one or more cells that express PTN, RPTP β/ζ and ALK.

In another aspect, the disclosure provides a method for inhibiting tumor growth in a subject comprising administering an effective amount of a medicament including an antibody against PTN or a fragment thereof wherein the tumor is comprised of one or more cells that express PTN, RPTP β/ζ and ALK.

In another aspect, the disclosure provides a method for reducing cancer cell proliferation in a subject comprising administering an effective amount of a medicament including an antibody against PTN or a fragment thereof wherein the cancer cell expresses PTN, RPTP β/ζ and ALK.

In another aspect, the disclosure provides a method for reducing cancer cell invasiveness comprising administering an effective amount of a medicament including an antibody against PTN or a fragment thereof wherein the cancer cell expresses PTN, RPTP β/ζ and ALK.

In another aspect, the disclosure provides a method for reducing cancer cell motility comprising administering an effective amount of a medicament including an antibody against PTN or a fragment thereof wherein the cancer cell expresses PTN, RPTP β/ζ and ALK.

In another aspect, the disclosure provides a method for reducing cancer cell metastasis comprising administering an effective amount of a medicament including an antibody against PTN or a fragment thereof wherein the cancer cell expresses PTN, RPTP β/ζ and ALK.

In another aspect, the disclosure provides a method for increasing the effectiveness of anticancer treatment of a cancer cell in a subject comprising administering chemotherapy treatment and administering an effective amount of a medicament including an antibody against PTN or a fragment thereof to the subject wherein the cancer cell expresses PTN, RPTP β/ζ and ALK.

In another aspect, the disclosure provides a method for increasing the effectiveness of anticancer treatment of a cancer cell in a subject comprising administering radiation therapy and administering an effective amount of a medicament including an antibody against PTN or a fragment thereof to the subject wherein the cancer cell expresses PTN, RPTP β/ζ and ALK.

In another aspect, the disclosure provides a method for reducing tumor cell angiogenesis in a subject comprising administering an effective amount of a medicament including an antibody against PTN or a fragment thereof wherein the tumor cell expresses PTN, RPTP β/ζ and ALK.

In another aspect, the disclosure provides a method for treating a cell that constitutively expresses PTN in a subject comprising administering an effective amount of an antibody against PTN or a fragment thereof wherein the cell expresses PTN, RPTP β/ζ and ALK.

In another aspect, the disclosure provides a method for treating a cell that expresses PTN constitutively in a subject comprising administering an effective amount of an antibody against PTN or a fragment thereof wherein the cell expresses PTN, RPTP β/ζ and ALK.

In another aspect, the disclosure provides a method for reducing the concentration of uncomplexed PTN in a cell of a subject comprising administering an effective amount of an antibody against PTN or a fragment thereof wherein the cell expresses PTN, RPTP β/ζ and ALK.

In another aspect, the disclosure provides a method for increasing the concentration uncomplexed monomeric RPTPβ/ζ in a cell of a subject comprising administering an effective amount of an antibody against PTN or a fragment thereof wherein the cell expresses PTN, RPTP β/ζ and ALK.

In another aspect, the disclosure provides a method for decreasing the concentration of (PTN)2-(RPTP β/ζ)2 heterotetramer in a cell of a subject comprising administering an effective amount of an antibody against PTN or a fragment thereof wherein the cell expresses PTN, RPTP β/ζ and ALK.

In another aspect, the disclosure provides a method for substantially reducing activity of ALK in a cell of a subject comprising administering an effective amount of an antibody against PTN or a fragment thereof wherein the cell expresses PTN, RPTP β/ζ and ALK.

In another aspect, the disclosure provides a method for reducing the activity of ALK in a cell of a subject comprising administering an effective amount of an antibody against PTN or a fragment thereof wherein the cell expresses PTN, RPTP β/ζ and ALK.

In another aspect, the disclosure provides a method for reducing the phosphorylation state of ALK in a cell of a subject comprising administering an effective amount of an antibody against PTN or a fragment thereof wherein the cell expresses PTN, RPTPβ/ζ and ALK, phospo-β-catenin (TYR-333), and phospho-γ-catenin.(TYR-550).

In another aspect, the disclosure provides a method for substantially deactivating Receptor Tyrosine-Protein Kinase erbB-4 (erbB-4) in a cell of a subject comprising administering an effective amount of an antibody against PTN or a fragment thereof wherein the cell expresses PTN, RPTP β/ζ and ALK.

In another aspect, the disclosure provides a method for reducing the activity of Receptor Tyrosine-Protein Kinase erbB-4 (erbB-4) in a cell of a subject comprising administering an effective amount of an antibody against PTN or a fragment thereof wherein the cell expresses PTN, RPTP β/ζ and ALK. In another aspect, the disclosure provides a method for reducing the phosphorylation state of Receptor Tyrosine-Protein Kinase erbB-4 (erbB-4) in a cell of a subject comprising administering an effective amount of an antibody against PTN or a fragment thereof wherein the cell expresses PTN, RPTP β/ζ and ALK.

In another aspect, the disclosure provides a method for substantially deactivating Protein Kinase C (PKC) in a cell of a subject comprising administering an effective amount of an antibody against PTN or a fragment thereof. wherein the cell expresses PTN, RPTP β/ζ and ALK.

In another aspect, the disclosure provides a method for reducing the activity of Protein Kinase C (PKC) in a cell of a subject comprising administering an effective amount of an antibody against PTN or a fragment thereof wherein the cell expresses PTN, RPTP β/ζ and ALK.

In another aspect, the disclosure provides a method for reducing the phosphorylation state of Protein Kinase C (PKC) in a cell of a subject comprising administering an effective amount of an antibody against PTN or a fragment thereof wherein the cell expresses PTN, RPTP β/ζ and ALK.

In another aspect, the disclosure provides a method for substantially deactivating Leukocyte Receptor Tyrosine Kinase (LTK) in a cell of a subject comprising administering an effective amount of an antibody against PTN or a fragment thereof wherein the cell expresses PTN, RPTP β/ζ and ALK.

In another aspect, the disclosure provides a method for reducing the activity of Leukocyte Receptor Tyrosine Kinase (LTK) in a cell of a subject comprising administering an effective amount of an antibody against PTN or a fragment thereof wherein the cell expresses PTN, RPTP β/ζ and ALK.

In another aspect, the disclosure provides a method for reducing the phosphorylation state of Leukocyte Receptor Tyrosine Kinase (LTK) in a cell of a subject comprising administering an effective amount of an antibody against PTN or a fragment thereof wherein the cell expresses PTN, RPTP β/ζ and ALK.

In another aspect, the disclosure provides a method for reducing phosphorylation of tyrosine-333 of β-catenin in a cell in a subject comprising administering an effective amount of an antibody against PTN or a fragment thereof wherein the cell expresses PTN, RPTP β/ζ and ALK.

In another aspect, the disclosure provides a method for increasing dephosphorylation of tyrosine-333 of β-catenin in a cell of a subject comprising administering an effective amount of an antibody against PTN or a fragment thereof wherein the cell expresses PTN, RPTP β/ζ and ALK.

In another aspect, the disclosure provides a method for reducing the phosphorylation state of tyrosine-333 of β-catenin in a cell of a subject comprising administering an effective amount of an antibody against PTN or a fragment thereof wherein the cell expresses PTN, RPTP β/ζ and ALK.

In another aspect, the disclosure provides a method for preventing disruption of cell-cell adhesion in a cell of a subject comprising administering an effective amount of an antibody against PTN or a fragment thereof wherein the cell expresses PTN, RPTP β/ζ and ALK.

In another aspect, the disclosure provides a method for inhibiting the reduction of cell-cell adhesion in a cell of a subject comprising administering an effective amount of an antibody against PTN or a fragment thereof wherein the cell expresses PTN, RPTP β/ζ and ALK.

In another aspect, the disclosure provides a method for reducing the disruption of a hydrogen bond between β-catenin and E-cadherin in a cell of a subject comprising administering an effective amount of an antibody against PTN or a fragment thereof wherein the cell expresses PTN, RPTP β/ζ and ALK.

In another aspect, the disclosure provides a method for reducing E-cadherin degradation by a cell's ubiquitin pathway in a cell of a subject comprising administering an effective amount of an antibody against PTN or a fragment thereof wherein the cell expresses PTN, RPTP β/ζ and ALK.

In another aspect, the disclosure provides a method for inhibiting upregulation of E-cadherin or N-cadherin in a cell of a subject comprising administering an effective amount of an antibody against PTN or a fragment thereof wherein the cell expresses PTN, RPTP β/ζ and ALK.

In another aspect, the disclosure provides a method for decreasing expression of E-cadherin or N-cadherin in a cell of a subject comprising administering an effective amount of an antibody against PTN or a fragment thereof wherein the cell expresses PTN, RPTP β/ζ and ALK.

In another aspect, the disclosure provides a method for decreasing levels of phospho-β-catenin (TYR-333) in a cell of a subject comprising administering an effective amount of an antibody against PTN or a fragment thereof wherein the cell expresses PTN, RPTP β/ζ and ALK.

In another aspect, the disclosure provides a method for decreasing expression or levels of β-catenin in a cell of a subject comprising administering an effective amount of an antibody against PTN or a fragment thereof wherein the cell expresses PTN, RPTP β/ζ and ALK.

In another aspect, the disclosure provides a method for inhibiting upregulation of at least one of integrin α1, α2, α4, and α5 in a cell of a subject comprising administering an effective amount of an antibody against PTN or a fragment thereof wherein the cell expresses PTN, RPTP β/ζ and ALK.

In another aspect, the disclosure provides a method for decreasing expression of at least one of integrin α1, α2, α4, and α5 in a cell of a subject comprising administering an effective amount of an antibody against PTN or a fragment thereof wherein the cell expresses PTN, RPTP β/ζ and ALK.

In another aspect, the disclosure provides a method for inhibiting downregulation of integrin α3 in a cell of a subject comprising administering an effective amount of an antibody against PTN or a fragment thereof wherein the cell expresses PTN, RPTP β/ζ and ALK.

In another aspect, the disclosure provides a method for increasing expression of integrin α3 in a cell of a subject comprising administering an effective amount of an antibody against PTN or a fragment thereof wherein the cell expresses PTN, RPTP β/ζ and ALK.

In another aspect, the disclosure provides a method for inhibiting downregulation of keratin 20 in a cell of a subject comprising administering an effective amount of an antibody against PTN or a fragment thereof wherein the cell expresses PTN, RPTP β/ζ and ALK.

In another aspect, the disclosure provides a method for increasing expression of keratin 20 in a cell of a subject comprising administering an effective amount of an antibody against PTN or a fragment thereof wherein the cell expresses PTN, RPTP β/ζ and ALK.

In another aspect, the disclosure provides a method for inhibiting upregulation of keratin 10 in a cell of a subject comprising administering an effective amount of an antibody against PTN or a fragment thereof wherein the cell expresses PTN, RPTP β/ζ and ALK.

In another aspect, the disclosure provides a method for decreasing expression of keratin 10 in a cell of a subject comprising administering an effective amount of an antibody against PTN or a fragment thereof wherein the cell expresses PTN, RPTP β/ζ and ALK.

In another aspect, the disclosure provides a method for reducing concentrations of β-catenin in the nucleus in a cell of a subject comprising administering an effective amount of an antibody against PTN or a fragment thereof. wherein the cell expresses PTN, RPTP β/ζ and ALK.

In another aspect, the disclosure provides a method for reducing import of β-catenin into the nucleus in a cell of a subject comprising administering an effective amount of an antibody against PTN or a fragment thereof wherein the cell expresses PTN, RPTP β/ζ and ALK.

In another aspect, the disclosure provides a method for reducing activation of epidermal growth factor receptor (EGFR) in a cell of a subject comprising administering an effective amount of an antibody against PTN or a fragment thereof wherein the cell expresses PTN, RPTP β/ζ and ALK.

In another aspect, the disclosure provides a method for increasing ubiquitination of β-catenin in a cell of a subject comprising administering an effective amount of an antibody against PTN or a fragment thereof wherein the cell expresses PTN, RPTP β/ζ and ALK.

In another aspect, the disclosure provides a method for modulating, for example reducing phosphorylation of EGFR in a cell of a subject comprising administering an effective amount of an antibody against PTN or a fragment thereof wherein the cell expresses PTN, RPTP β/ζ and ALK.

In another aspect, the disclosure provides a method for reducing the transcription of genes induced by Tcf/Lef family in a cell of a subject comprising administering an effective amount of an antibody against PTN or a fragment thereof wherein the cell expresses PTN, RPTP β/ζ and ALK.

In another aspect, the disclosure provides a method for reducing activation of Akt in a cell of a subject comprising administering an effective amount of an antibody against PTN or a fragment thereof wherein the cell expresses PTN, RPTP β/ζ and ALK.

In another aspect, the disclosure provides a method for reducing phosphorylation of Akt in a cell of a subject comprising administering an effective amount of an antibody against PTN or a fragment thereof wherein the cell expresses PTN, RPTP β/ζ and ALK.

In another aspect, the disclosure provides a method for increasing activation of GSK3β (Glycogen Synthase Kinase 3β) in a cell of a subject comprising administering an effective amount of an antibody against PTN or a fragment thereof wherein the cell expresses PTN, RPTPβ/ζ and ALK.

In another aspect, the disclosure provides a method for decreasing phosphorylation of GSK3β in a cell of a subject comprising administering an effective amount of an antibody against PTN or a fragment thereof wherein the cell expresses PTN, RPTPβ/ζ and ALK.

In another aspect, the disclosure provides a method for increasing phosphorylation of one or more of serine 33, serine 37, and threonine 41 in β-catenin in a cell of a subject comprising administering an effective amount of an antibody against PTN or a fragment thereof wherein the cell expresses PTN, RPTP γ/ζ and ALK.

In another aspect, the disclosure provides a method for reducing the concentration of γ-catenin in the nucleus in a cell of a subject comprising administering an effective amount of an antibody against PTN or a fragment thereof wherein the cell expresses PTN, RPTP β/ζ and ALK.

In another aspect, the disclosure provides a method for downregulating expression and/or decreasing levels of one or more downstream targets of the PTN/RPTPβ/ζ signaling pathway including at least MDR1, ZO1, uPAR, c-jun, survivin, DRCTNNB1A, PPAR ∝, Id2, TCF-1, Brachyury, NBL4, c-myc, and ITF-2 in a cell of a subject comprising administering an effective amount of an antibody against PTN or a fragment thereof wherein the cell expresses PTN, RPTP β/ζ and ALK.

In another aspect, the disclosure provides a method for upregulating expression and/or increasing levels of one or more of Cyclin D1, Fra-1 and Connexin-43 in a cell of a subject comprising administering an effective amount of an antibody against PTN or a fragment thereof wherein the cell expresses PTN, RPTP β/ζ and ALK.

In another aspect, the disclosure provides a method for reducing serine phosphorylation of β-adducin (adducin 2β) in a cell of a subject comprising administering an effective amount of an antibody against PTN or a fragment thereof wherein the cell expresses PTN, RPTP β/ζ and ALK.

In another aspect, the disclosure provides a method for reducing the phosphorylation of GIT1/Cat-1 in a cell of a subject comprising administering an effective amount of an antibody against PTN or a fragment thereof wherein the cell expresses PTN, RPTP β/ζ and ALK.

In another aspect, the disclosure provides a method for reducing the phosphorylation of P190RhoGAP in a cell of a subject comprising administering an effective amount of an antibody against PTN or a fragment thereof wherein the cell expresses PTN, RPTP β/ζ and ALK.

In another aspect, the disclosure provides a method for reducing phosphorylation of HDAC-2 in a cell of a subject comprising administering an effective amount of an antibody against PTN or a fragment thereof wherein the cell expresses PTN, RPTP β/ζ and ALK.

In another aspect, the disclosure provides a method for reducing phosphorylation of FYN in a cell of a subject comprising administering an effective amount of an antibody against PTN or a fragment thereof wherein the cell expresses PTN, RPTP β/ζ and ALK.

In another aspect, the disclosure provides a method for modulating, for example decreasing steady-state phosphorylation levels of molecular targets of RPTP β/ζ in a cell of a subject comprising administering an effective amount of an antibody against PTN or a fragment thereof wherein the cell expresses PTN, RPTP β/ζ and ALK.

In another aspect, the disclosure provides a method for modulating, for example decreasing steady-state phosphorylation levels of molecular targets of ALK in a cell of a subject comprising administering an effective amount of a medicament including an antibody against PTN or a fragment thereof wherein the cell expresses PTN, RPTP β/ζ and ALK.

In another aspect, the disclosure provides a method for preventing a cell of a subject from progressing to a malignant cancer cell such as for example a breast cancer cell comprising administering an effective amount of a medicament including an antibody against PTN or a fragment thereof wherein the cell expresses PTN, RPTP β/ζ and ALK.

In another aspect, the disclosure provides a method for reducing deposition of collagen by a tumor cell or tumor fibroblast comprising administering an effective amount of a medicament including an antibodyagainst PTN or a fragment thereof wherein the cell expresses PTN, RPTP β/ζ and ALK.

In another aspect, the disclosure provides a method for reducing deposition of or tumor-associated fibroblast by a tumor cell comprising administering an effective amount of a medicament including an antibodyagainst PTN or a fragment thereof wherein the cell expresses PTN, RPTP β/ζ and ALK.

In another aspect, the disclosure provides a method for reducing secretion of collagen by a tumor cell or tumor-associated fibroblast comprising administering an effective amount of a medicament including an antibodyagainst PTN or a fragment thereof wherein the cell expresses PTN, RPTP β/ζ and ALK.

In another aspect, the disclosure provides a method for reducing secretion of elastin by a tumor cell or tumor-associated fibroblast comprising administering an effective amount of a medicament including an antibodyagainst PTN or a fragment thereof wherein the cell expresses PTN, RPTP β/ζ and ALK.

In another aspect, the disclosure provides a method for reducing the incidence of cancer in a subject comprising administering an effective amount of a medicament including an antibodyagainst PTN or a fragment thereof wherein the cell expresses PTN, RPTP β/ζ and ALK.

In another aspect, the disclosure provides a method for reducing the malignancy of a cancer in a subject comprising administering an effective amount of a medicament including an antibodyagainst PTN or a fragment thereof wherein the cell expresses PTN, RPTP β/ζ and ALK.

In another aspect, the disclosure provides a method for reducing scirrhous patterned carcinoma type breast cancer in a subject comprising administering an effective amount of a medicament including an antibodyagainst PTN or a fragment thereof wherein the cell expresses PTN, RPTP β/ζ and ALK.

In another aspect, the disclosure provides a method for reversing EMT in a cell of a subject comprising administering an effective amount of a medicament including an antibodyagainst PTN or a fragment thereof wherein the cell expresses PTN, RPTP β/ζ and ALK.

In another aspect, the disclosure provides a method for preventing a cell from undergoing an EMT in a subject comprising administering an effective amount of a medicament including an antibodyagainst PTN or a fragment thereof wherein the cell expresses PTN, RPTP β/ζ and ALK.

In an aspect the disclosure provides methods for reducing activation of Insulin-like Growth Factor 1 Receptor (IGF-1 Receptor; IGFR-1) in a cell of a subject comprising administering an effective amount of an antibody against PTN or a fragment thereof wherein the cell expresses PTN, RPTP β/ζ and ALK.

In an aspect the disclosure provides methods for reducing phosphorylation of Insulin-like Growth Factor 1 Receptor (IGF-1 Receptor; IGFR-1) in a cell of a subject comprising administering an effective amount of an antibody against PTN or a fragment thereof wherein the cell expresses PTN, RPTP β/ζ and ALK.

In an aspect the disclosure provides methods for reducing activation of Human Epidermal Growth Factor Receptor 2 (HER2/neu, also known as ErbB-2) in a cell of a subject comprising administering an effective amount of an antibody against PTN or a fragment thereof wherein the cell expresses PTN, RPTP β/ζ and ALK.

In an aspect the disclosure provides methods for reducing phosphorylation of Human Epidermal Growth Factor Receptor 2 (HER2/neu, also known as ErbB-2) in a cell of a subject comprising administering an effective amount of an antibody against PTN or a fragment thereof wherein the cell expresses PTN, RPTP β/ζ and ALK.

In an aspect the disclosure provides methods for reducing activation of Vascular Endothelial Growth Factor Receptor 1 (VEGFR-1, Flt-1) in a cell of a subject comprising administering an effective amount of an antibody against PTN or a fragment thereof wherein the cell expresses PTN, RPTP β/ζ and ALK.

In an aspect the disclosure provides methods for reducing phosphorylation of Vascular Endothelial Growth Factor Receptor 1 (VEGFR-1, Flt-1) in a cell of a subject comprising administering an effective amount of an antibody against PTN or a fragment thereof wherein the cell expresses PTN, RPTP β/ζ and ALK.

In an aspect the disclosure provides methods for reducing activation of Vascular Endothelial Growth Factor Receptor 2 (VEGFR-2, KDR/Flk-1) in a cell of a subject comprising administering an effective amount of an antibody against PTN or a fragment thereof wherein the cell expresses PTN, RPTPβ/ζ and ALK.

In an aspect the disclosure provides methods for reducing phosphorylation of Vascular Endothelial Growth Factor Receptor 2 (VEGFR-2, KDR/Flk-1) in a cell of a subject comprising administering an effective amount of an antibody against PTN or a fragment thereof wherein the cell expresses PTN, RPTP β/ζ and ALK.

In an aspect the disclosure provides methods for reducing activation of VEGFR-3 in a cell of a subject comprising administering an effective amount of an antibody against PTN or a fragment thereof wherein the cell expresses PTN, RPTP β/ζ and ALK.

In an aspect the disclosure provides methods for reducing phosphorylation of VEGFR-3 in a cell of a subject comprising administering an effective amount of an antibody against PTN or a fragment thereof wherein the cell expresses PTN, RPTP β/ζ and ALK.

The disclosure also provides for each of the methods herein that when an effective amount of an antibody against PTN or a fragment thereof is administered in combination with one or more of the negative PTN, and decoy RPTP β/ζ, the antibody against PTN is a monoclonal antibody or fragment thereof.

The disclosure also provides for each of the methods herein that wherein when an effective amount of an antibody against PTN or a fragment thereof is administered in combination with one or more of the negative PTN, and decoy RPTP β/ζ, the antibody against PTN is a polyclonal antibody or fragment thereof.

The disclosure also provides for each of the methods herein that when an effective amount of an antibody against PTN or a fragment thereof is administered in combination with one or more of the negative PTN, decoy RPTP β/ζ, the antibody against PTN is a humanized antibody or fragment thereof.

The disclosure also provides for each of the methods herein described herein that an effective amount decoy RPTP β/ζ or an effective amount of negative PTN may be used instead of an effective amount of an antibody against PTN or a fragment thereof.

The disclosure also provides for each of the methods herein that wherein when an effective amount of an antibody against PTN or a fragment thereof is administered in combination with one or more of the negative PTN, and decoy RPTP β/ζ, the antibody against PTN is a polyclonal antibody or fragment thereof.

The disclosure also provides for each of the methods herein that when an effective amount of an antibody against PTN or a fragment thereof is administered in combination with one or more of the negative PTN, decoy RPTP β/ζ, the antibody against PTN is a humanized antibody or fragment thereof.

The disclosure also provides for each of the methods herein that when an effective amount of an antibody against PTN or a fragment thereof is administered in combination with one or more of the negative PTN, and decoy RPTP β/ζ, the antibody against PTN is a monoclonal antibody or fragment thereof, and wherein when an effective amount of antibody against PTN is administered to the subject, the antibody substantially binds to PTN and RPTP β/ζ is consequently no longer substantially inactivated, and wherein when an effective amount of negative PTN is administered to the subject, the negative PTN monomers substantially bind to endogenous PTN monomers to form substantially inactive heterodimers and RPTP β/ζ is consequently no longer substantially inactivated, and wherein when an effective amount of decoy RPTP β/ζ is administered to the subject, one or more dimers of endogenous PTN binds to the decoy RPTP β/ζ and RPTP β/ζ is consequently no longer substantially inactivated.

The disclosure also provides for each of the methods herein that wherein when an effective amount of an antibody against PTN or a fragment thereof is administered in combination with one or more of the negative PTN, and decoy RPTP β/ζ, the antibody against PTN is a polyclonal antibody or fragment thereof, and wherein when an effective amount of antibody against PTN is administered to the subject, the antibody substantially binds to PTN and RPTP β/ζ is consequently no longer substantially inactivated, and wherein when an effective amount of negative PTN is administered to the subject, the negative PTN monomers substantially bind to endogenous PTN monomers to form substantially inactive heterodimers and RPTP β/ζ is consequently no longer substantially inactivated, and wherein when an effective amount of decoy RPTP β/ζ is administered to the subject, one or more dimers of endogenous PTN binds to the decoy RPTP β/ζ and RPTP β/ζ is consequently no longer substantially inactivated.

The disclosure also provides for each of the methods herein that when an effective amount of an antibody against PTN or a fragment thereof is administered in combination with one or more of the negative PTN, decoy RPTP β/ζ, the antibody against PTN is a humanized antibody or fragment thereof, and wherein when an effective amount of antibody against PTN is administered to the subject, the antibody substantially binds to PTN and RPTP β/ζ is consequently no longer substantially inactivated, and wherein when an effective amount of negative PTN is administered to the subject, the negative PTN monomers substantially bind to endogenous PTN monomers to form substantially inactive heterodimers and RPTP β/ζ is consequently no longer substantially inactivated, and wherein when an effective amount of decoy RPTP β/ζ is administered to the subject, one or more dimers of endogenous PTN binds to the decoy RPTP β/ζ and RPTP β/ζ is consequently no longer substantially inactivated.

The disclosure also provides for each of the methods herein that when an effective amount of an antibody against PTN or a fragment thereof is administered in combination with one or more of the negative PTN, and decoy RPTP β/ζ, the antibody against PTN is a monoclonal antibody or fragment thereof, and wherein when an effective amount of antibody against PTN is administered to the subject, the antibody substantially binds to PTN, RPTP β/ζ is consequently no longer substantially inactivated and ALK is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTPβ/ζ is consequently no longer substantially inactivated and ALK is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and ALK is consequently substantially dephosphorylated by RPTP β/ζ.

The disclosure also provides for each of the methods herein that wherein when an effective amount of an antibody against PTN or a fragment thereof is administered in combination with one or more of the negative PTN, and decoy RPTP β/ζ, the antibody against PTN is a polyclonal antibody or fragment thereof, and wherein when an effective amount of antibody against PTN is administered to the subject, the antibody substantially binds to PTN, RPTP β/ζ is consequently no longer substantially inactivated and ALK is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and ALK is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and ALK is consequently substantially dephosphorylated by RPTP β/ζ.

The disclosure also provides for each of the methods herein that when an effective amount of an antibody against PTN or a fragment thereof is administered in combination with one or more of the negative PTN, decoy RPTP β/ζ, the antibody against PTN is a humanized antibody or fragment thereof, and wherein when an effective amount of antibody against PTN is administered to the subject, the antibody substantially binds to PTN, RPTP β/ζ is consequently no longer substantially inactivated and ALK is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and ALK is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and ALK is consequently substantially dephosphorylated by RPTP β/ζ.

In another aspect, the disclosure provides a method for treating cancer in a subject comprising administering an effective amount of a medicament including an antibody against PTN or a fragment thereof wherein the cancer is comprised of one or more cells that express PTN, RPTP β/ζ and one or more of EGFR, IGFR-1, erbB2, erbB4, PKC, LTK and VEGFR.

In another aspect, the disclosure provides a method for reducing risk of developing cancer in a subject comprising administering an effective amount of an antibody against PTN or a fragment thereof wherein the cancer is comprised of one or more cells that express PTN, RPTP β/ζ and one or more of EGFR, IGFR-1, erbB2, erbB4, PKC, LTK and VEGFR.

In another aspect, the disclosure provides a method for diminishing the likelihood of development of a cancer in a subject comprising administering an effective amount of an antibody against PTN or a fragment thereof wherein the cancer is comprised of one or more cells that express PTN, RPTP β/ζ and one or more of EGFR, IGFR-1, erbB2, erbB4, PKC, LTK and VEGFR.

In another aspect, the disclosure provides a method for reversing tumor growth in a subject comprising administering an effective amount of an antibody against PTN or a fragment thereof wherein the tumor is comprised of one or more cells that express PTN, RPTP β/ζ and one or more of EGFR, IGFR-1, erbB2, erbB4, PKC, LTK and VEGFR.

In another aspect, the disclosure provides a method for inhibiting tumor growth in a subject comprising administering an effective amount of an antibody against PTN or a fragment thereof wherein the tumor is comprised of one or more cells that express PTN, RPTP β/ζ and one or more of EGFR, IGFR-1, erbB2, erbB4, PKC, LTK and VEGFR.

In another aspect, the disclosure provides a method for reducing cancer cell proliferation in a subject comprising administering an effective amount of an antibody against PTN or a fragment thereof wherein the cancer cell expresses PTN, RPTP β/ζ and one or more of EGFR, IGFR-1, erbB2, erbB4, PKC, LTK and VEGFR.

In another aspect, the disclosure provides a method for reducing cancer cell invasiveness comprising administering an effective amount of an antibody against PTN or a fragment thereof wherein the cancer cell expresses PTN, RPTP β/ζ and one or more of EGFR, IGFR-1, erbB2, erbB4, PKC, LTK and VEGFR.

In another aspect, the disclosure provides a method for reducing cancer cell motility comprising administering an effective amount of an antibody against PTN or a fragment thereof wherein the cancer cell expresses PTN, RPTP β/ζ and one or more of EGFR, IGFR-1, erbB2, erbB4, PKC, LTK and VEGFR.

In another aspect, the disclosure provides a method for reducing cancer cell metastasis comprising administering an effective amount of an antibody against PTN or a fragment thereof wherein the cancer cell expresses PTN, RPTP β/ζ and one or more of EGFR, IGFR-1, erbB2, erbB4, PKC, LTK and VEGFR.

In another aspect, the disclosure provides a method for increasing the effectiveness of anticancer treatment of a cancer cell in a subject comprising administering chemotherapy treatment and administering an effective amount of an antibody against PTN or a fragment thereof to the subject wherein the cancer cell expresses PTN, RPTP β/ζ and one or more of EGFR, IGFR-1, erbB2, erbB4, PKC, LTK and VEGFR.

In another aspect, the disclosure provides a method for increasing the effectiveness of anticancer treatment of a cancer cell in a subject comprising administering radiation therapy and administering an effective amount of an antibody against PTN or a fragment thereof to the subject wherein the cancer cell expresses PTN, RPTP β/ζ and one or more of EGFR, IGFR-1, erbB2, erbB4, PKC, LTK and VEGFR.

In another aspect, the disclosure provides a method for reducing tumor cell angiogenesis in a subject comprising administering an effective amount of a medicament including an antibody against PTN or a fragment thereof wherein the tumor cell expresses PTN, RPTP β/ζ and one or more of EGFR, IGFR-1, erbB2, erbB4, PKC, LTK and VEGFR.

In another aspect, the disclosure provides a method for treating a cell that constitutively expresses PTN in a subject comprising administering an effective amount of a medicament including an antibodyagainst PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for treating a cell that expresses PTN constitutively in a subject comprising administering an effective amount of a medicament including an antibodyagainst PTN or a fragment thereof wherein the cell expresses PTN, RPTPβ/ζ and one or more of EGFR, IGFR-1, erbB2, erbB4, PKC, LTK and VEGFR.

In another aspect, the disclosure provides a method for reducing the concentration of uncomplexed PTN in a cell of a subject comprising administering an effective amount of a medicament including an antibodyagainst PTN or a fragment thereof wherein the cell expresses PTN, RPTPβ/ζ and one or more of EGFR, IGFR-1, erbB2, erbB4, PKC, LTK and VEGFR.

In another aspect, the disclosure provides a method for increasing the concentration uncomplexed RPTPβ/ζ in a cell of a subject comprising administering an effective amount of a medicament including an antibodyagainst PTN or a fragment thereof wherein the cell expresses PTN, RPTPβ/ζ and one or more of EGFR, IGFR-1, erbB2, erbB4, PKC, LTK and VEGFR.

In another aspect, the disclosure provides a method for decreasing the concentration of (PTN)2-(RPTP β/ζ)2 heterotetramer in a cell of a subject comprising administering an effective amount of a medicament including an antibodyagainst PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for substantially reducing activity of ALK in a cell of a subject comprising administering an effective amount of a medicament including an antibodyagainst PTN or a fragment thereof wherein the cell expresses PTN, RPTPβ/ζ and one or more of EGFR, IGFR-1, erbB2, erbB4, PKC, LTK and VEGFR.

In another aspect, the disclosure provides a method for reducing the activity of ALK in a cell of a subject comprising administering an effective amount of a medicament including an antibodyagainst PTN or a fragment thereof wherein the cell expresses PTN, RPTPβ/ζ and one or more of EGFR, IGFR-1, erbB2, erbB4, PKC, LTK and VEGFR.

In another aspect, the disclosure provides a method for reducing the phosphorylation state of ALK in a cell of a subject comprising ragainst PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for substantially deactivating Receptor Tyrosine-Protein Kinase erbB-4 (erbB-4) in a cell of a subject comprising administering an effective amount of a medicament including an antibodyagainst PTN or a fragment thereof wherein cell expresses PTN, RPTP β/ζ and one or more of EGFR, IGFR-1, erbB2, erbB4, PKC, LTK and VEGFR.

In another aspect, the disclosure provides a method for reducing the activity of Receptor Tyrosine-Protein Kinase erbB-4 (erbB-4) in a cell of a subject comprising administering an effective amount of a medicament including an antibodyagainst PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing the phosphorylation state of Receptor Tyrosine-Protein Kinase erbB-4 (erbB-4) in a cell of a subject comprising administering an effective amount of a medicament including an antibodyagainst PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for substantially deactivating Protein Kinase C (PKC) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing the activity of Protein Kinase C (PKC) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing the phosphorylation state of Protein Kinase C (PKC) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for substantially deactivating Leukocyte Receptor Tyrosine Kinase (LTK) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing the activity of Leukocyte Receptor Tyrosine Kinase (LTK) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing the phosphorylation state of Leukocyte Receptor Tyrosine Kinase (LTK) in a cell of a subject comprising administering an effective amount of an antibody against PTN or a fragment thereof wherein the cell expresses PTN, RPTP β/ζ and one or more of EGFR, IGFR-1, erbB2, erbB4, PKC, LTK and VEGFR.

In another aspect, the disclosure provides a method for reducing phosphorylation of tyrosine-333 of β-catenin in a cell in a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing dephosphorylation of tyrosine-333 of β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the phosphorylation state of tyrosine-333 of β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for preventing disruption of cell-cell adhesion in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting the reduction of cell-cell adhesion in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the disruption of a hydrogen bond between β-catenin and E-cadherin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing E-cadherin degradation by a cell's ubiquitin pathway in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting upregulation of E-cadherin or N-cadherin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing expression of E-cadherin or N-cadherin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing the phosphorylation of tyrosine-333 of β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing expression or levels of β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting upregulation of at least one of integrin α1, α2, α4, and α5 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing expression of at least one of integrin α1, α2, α4, and α5 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting downregulation of integrin α3 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing expression of integrin α3 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting downregulation of keratin 20 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing expression of keratin 20 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for inhibiting upregulation of keratin 10 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing expression of keratin 10 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing concentrations of β-catenin in the nucleus in a cell of a subject comprising administering an effective amount of an antibody against PTN or a fragment thereof. wherein the cell expresses PTN, RPTP β/ζ, ALK and one or more of EGFR, IGFR-1, erbB2, erbB4, PKC, LTK and VEGFR.

In another aspect, the disclosure provides a method for reducing import of β-catenin into the nucleus in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing activation of epidermal growth factor receptor (EGFR) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing ubiquitination of β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for modulating, for example reducing phosphorylation of EGFR in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the transcription of genes induced by Tcf/Lef family in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing activation of Akt in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing phosphorylation of Akt in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing activation of GSK3β (Glycogen Synthase Kinase 3β) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for decreasing phosphorylation of GSK3β in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for increasing phosphorylation of one or more of serine 33, serine 37, and threonine 41 in β-catenin in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the concentration of γ-catenin in the nucleus in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for downregulating expression and/or decreasing levels of one or more downstream targets of the PTN/RPTPβ/ζ signaling pathway including at least MDR1, ZO1, uPAR, c-jun, survivin, DRCTNNB1A, PPAR ∝, Id2, TCF-1, Brachyury, NBL4, c-myc, and ITF-2 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for upregulating expression and/or increasing levels of one or more of Cyclin D1, Fra-1 and Connexin-43 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing serine phosphorylation of β-adducin (adducin 2β) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the phosphorylation of GIT1/Cat-1 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the phosphorylation of P190RhoGAP in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing phosphorylation of HDAC-2 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing phosphorylation of FYN in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for modulating, for example decreasing steady-state phosphorylation levels of molecular targets of RPTP β/ζ in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for modulating, for example decreasing steady-state phosphorylation levels of molecular targets of ALK in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for preventing a cell of a subject from progressing to a malignant cancer cell such as, for example a breast cancer cell comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing deposition of or tumor-associated fibroblast by a tumor cell comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing deposition of elastin by a tumor cell or tumor-associated fibroblast comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing secretion of collagen by a tumor cell or tumor-associated fibroblast comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing secretion of elastin by a tumor cell tumor-associated fibroblast comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the incidence of cancer in a subject comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing the malignancy of a cancer in a subject comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reducing scirrhous patterned carcinoma type breast cancer in a subject comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for reversing EMT in a cell of a subject comprising administering an effective amount of a medicament including an antibodyagainst PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for preventing a cell from undergoing an EMT in a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing activation of Insulin-like Growth Factor 1 Receptor (IGF-1 Receptor; IGFR-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing phosphorylation of Insulin-like Growth Factor 1 Receptor (IGF-1 Receptor; IGFR-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing activation of Human Epidermal Growth Factor Receptor 2 (HER2/neu, also known as ErbB-2) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing phosphorylation of Human Epidermal Growth Factor Receptor 2 (HER2/neu, also known as ErbB-2) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing activation of Vascular Endothelial Growth Factor Receptor 1 (VEGFR-1, Flt-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing phosphorylation of Vascular Endothelial Growth Factor Receptor 1 (VEGFR-1, Flt-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing activation of Vascular Endothelial Growth Factor Receptor 2 (VEGFR-2, KDR/Flk-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing phosphorylation of Vascular Endothelial Growth Factor Receptor 2 (VEGFR-2, KDR/Flk-1) in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing activation of VEGFR-3 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In an aspect the disclosure provides methods for reducing phosphorylation of VEGFR-3 in a cell of a subject comprising administering an effective amount of an antibody against PTN or a fragment thereof wherein the cell expresses PTN, RPTP β/ζ, ALK and one or more of EGFR, IGFR-1, erbB2, erbB4, PKC, LTK and VEGFR.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for increasing the effectiveness of anticancer treatment of a cancer cell in a subject comprising administering to the subject chemotherapy treatment and, in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTPβ/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTPβ/ζ and one or more of EGFR, IGFR-1, erbB2, erbB4, PKC, LTK and VEGFR and wherein when an effective amount of antibody against PTN is administered to the subject, the antibody substantially binds to PTN, RPTPβ/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTPβ/ζ is administered to the subject, one or more dimers of endogenous PTN binds to the decoy RPTP β/ζ, RPTPβ/ζ is consequently no longer substantially inactivated and one or more of EGFR, IGFR-1, erbB2, erbB4, PKC, LTK and VEGFR is consequently substantially dephosphorylated by RPTPβ/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for substantially deactivating Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the activity of Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the phosphorylation state of Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for substantially deactivating Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the activity of Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the phosphorylation state of Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for substantially deactivating Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the activity of Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for reducing the phosphorylation state of Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and one or more of EGFR, IGFR-1, erbB2, erbB4, PKC, LTK and VEGFR and wherein when an effective amount of antibody against PTN is administered to the subject, the antibody substantially binds to PTN, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of negative PTN is administered to the subject, the negative PTN monomers substantially bind to endogenous PTN monomers to form substantially inactive heterodimers, RPTP β/ζ is consequently no longer substantially inactivated and one or more of EGFR, IGFR-1, erbB2, erbB4, PKC, LTK and VEGFR is consequently substantially dephosphorylated by RPTP β/ζ, and wherein when an effective amount of decoy RPTP β/ζ is administered to the subject, one or more dimers of endogenous PTN binds to the decoy RPTP β/ζ, RPTP β/ζ is consequently no longer substantially inactivated and one or more of EGFR, IGFR-1, erbB2, erbB4, PKC, LTK and VEGFR is consequently substantially dephosphorylated by RPTP β/ζ.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering alone or in combination an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, Epidermal Growth Factor Receptor (EGFR) is consequently substantially dephosphorylated by RPTP β/ζ.

In another aspect, the disclosure provides a method for substantially deactivating EGFR in a cell of a subject comprising administering alone or in combination an effective amount of an antibody against PTN or a fragment thereof, an effective amount of negative PTN or decoy RPTPβ/ζ wherein cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, EGFR is consequently substantially dephosphorylated by RPTP β/ζ.

In another aspect, the disclosure provides a method for reducing the activity of EGFR in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, EGFR is consequently substantially dephosphorylated by RPTP β/ζ.

In another aspect, the disclosure provides a method for reducing the phosphorylation state of EGFR in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, EGFR is consequently substantially dephosphorylated by RPTP β/ζ.

In another aspect, the disclosure provides a method for treating cancer in a subject comprising administering alone or in combination an effective amount of an antibody against PTN or a fragment thereof, an effective amount of negative PTN or decoy RPTPβ/ζ wherein the cancer is comprised of one or more cells that express PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, IGFR-1 is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for increasing the effectiveness of anticancer treatment of a cancer cell in a subject comprising administering to the subject chemotherapy and in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for treating a cell that constitutively expresses PTN in a subject comprising administering a medicament including, alone, or in combination, an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for treating a cell that expresses PTN constitutively in a subject comprising administering a medicament including, alone, or in combination, an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTPβ/ζ is administered to the subject, Insulin-like Growth Factor 1 Receptor (IGFR-1) is consequently substantially dephosphorylated by RPTP β/ζ.

In another aspect, the disclosure provides a method for reducing the concentration of uncomplexed PTN in a cell of a subject comprising administering a medicament including, alone, or in combination, an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and IGFR-1 and wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and IGFR-land wherein when one or more of an effective amount of antibody against PTN is administered to the subject, an effective amount of negative PTN is administered to the subject, and an effective amount of decoy RPTP β/ζ is administered to the subject, Insulin-like Growth Factor 1 Receptor (IGFR-1) is consequently substantially dephosphorylated by RPTP β/ζ.

In another aspect, the disclosure provides a method for substantially deactivating IGFR-1 in a cell of a subject comprising administering alone or in combination an effective amount of an antibody against PTN or a fragment thereof, an effective amount of negative PTN or decoy RPTPβ/ζ wherein cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, IGFR-1 is consequently substantially dephosphorylated by RPTP β/ζ.

In another aspect, the disclosure provides a method for reducing the activity of IGFR-1 in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, IGFR-1 is consequently substantially dephosphorylated by RPTP β/ζ.

In another aspect, the disclosure provides a method for reducing the phosphorylation state of IGFR-1 in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, IGFR-1is consequently substantially dephosphorylated by RPTP β/ζ.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for treating a cell that constitutively expresses PTN in a subject comprising administering a medicament including, alone, or in combination, an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for substantially deactivating erbB2 in a cell of a subject comprising administering alone or in combination an effective amount of an antibody against PTN or a fragment thereof, an effective amount of negative PTN or decoy RPTPβ/ζ wherein cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing the activity of erbB2 in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for reducing the phosphorylation state of erbB2 in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB2 and wherein when one or more of an effective amount of antibody against PTN is administered to the subject, an effective amount of negative PTN is administered to the subject, and an effective amount of decoy RPTP β/ζ is administered to the subject, Human Epidermal Growth Factor Receptor 2 (erbB2; HER2/neu) is consequently substantially dephosphorylated by RPTP β/ζ.

In another aspect, the disclosure provides a method for treating cancer in a subject comprising administering alone or in combination an effective amount of an antibody against PTN or a fragment thereof, an effective amount of negative PTN or decoy RPTPβ/ζ wherein the cancer is comprised of one or more cells that express PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing risk of developing cancer in a subject comprising administering alone or in combination an effective amount of an antibody against PTN or a fragment thereof, an effective amount of negative PTN or decoy RPTPβ/ζ wherein the cancer is comprised of one or more cells that express PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for diminishing the likelihood of development of a cancer in a subject comprising administering alone or in combination an effective amount of an antibody against PTN or a fragment thereof, an effective amount of negative PTN or decoy RPTPβ/ζ wherein the cancer is comprised of one or more cells that express PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reversing tumor growth in a subject comprising administering alone or in combination an effective amount of an antibody against PTN or a fragment thereof, an effective amount of negative PTN or decoy RPTPβ/ζ wherein the tumor is comprised of one or more cells that express PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for inhibiting tumor growth in a subject comprising administering alone or in combination an effective amount of an antibody against PTN or a fragment thereof, an effective amount of negative PTN or decoy RPTPβ/ζ wherein the tumor is comprised of one or more cells that express PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing cancer cell proliferation in a subject comprising administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing cancer cell invasiveness comprising administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing cancer cell motility comprising administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing cancer cell metastasis comprising administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for increasing the susceptibility of a cancer cell to radiation therapy in a subject comprising administering alone or in combination an effective amount of an antibody against PTN or a fragment thereof, an effective amount of negative PTN or decoy RPTPβ/ζ wherein the cancer cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing tumor cell angiogenesis in a subject comprising administering alone or in combination an effective amount of an antibody against PTN or a fragment thereof, an effective amount of negative PTN or decoy RPTPβ/ζ wherein the tumor cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for increasing the concentration uncomplexed monomeric RPTPβ/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and EGFR and wherein when one or more of an effective amount of antibody against PTN is erbB4 to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for substantially deactivating Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein cell expresses PTN, RPTP β/ζ and EGFR and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing the activity of Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for reducing the phosphorylation state of Receptor Tyrosine-Protein Kinase erbB-4 (erbB-4) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and erbB4 and wherein when one or more of an effective amount of antibody against PTN is administered to the subject, an effective amount of negative PTN is administered to the subject, and an effective amount of decoy RPTP β/ζ is administered to the subject, erbB4 is consequently substantially dephosphorylated by RPTP β/ζ.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for substantially deactivating Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing the activity of Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for reducing the phosphorylation state of Protein Kinase C (PKC) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and PKC and wherein when one or more of an effective amount of antibody against PTN is administered to the subject, an effective amount of negative PTN is administered to the subject, and an effective amount of decoy RPTP β/ζ is administered to the subject, PKC is consequently substantially dephosphorylated by RPTP β/ζ.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTPβ/ζ is administered to the subject, LTK is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for substantially deactivating Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing the activity of Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for reducing the phosphorylation state of Leukocyte Receptor Tyrosine Kinase (LTK) in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and LTK and wherein when one or more of an effective amount of antibody against PTN is administered to the subject, an effective amount of negative PTN is administered to the subject, and an effective amount of decoy RPTP β/ζ is administered to the subject, LTK is consequently substantially dephosphorylated by RPTP β/ζ.

In another aspect, the disclosure provides a method for treating cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing risk of developing cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for diminishing the likelihood of development of a cancer in a subject comprising administering a medicament including, alone, or in combination, an effective amount of an antibody against PTN or a fragment thereof, an effective amount of negative PTN or decoy RPTPβ/ζ wherein the cancer is comprised of one or more cells that express PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reversing tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for inhibiting tumor growth in a subject comprising administering a medicament including, alone, or in combination, an effective amount of an antibody against PTN or a fragment thereof, an effective amount of negative PTN or decoy RPTPβ/ζ wherein the tumor is comprised of one or more cells that express PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing cancer cell proliferation in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing cancer cell invasiveness comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing cancer cell motility comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing cancer cell metastasis comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for increasing the effectiveness of anticancer treatment of a cancer cell in a subject comprising administering to the subject chemotherapy treatment and in addition, administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for increasing the susceptibility of a cancer cell to radiation therapy in a subject comprising administering a medicament including, alone, or in combination, an effective amount of an antibody against PTN or a fragment thereof, an effective amount of negative PTN or decoy RPTPβ/ζ wherein the cancer cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing tumor cell angiogenesis in a subject comprising administering a medicament including, alone, or in combination, an effective amount of an antibody against PTN or a fragment thereof, an effective amount of negative PTN or decoy RPTPβ/ζ wherein the tumor cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for treating a cell that constitutively expresses PTN in a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for treating a cell that expresses PTN constitutively in a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing the concentration of uncomplexed PTN in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for increasing the concentration uncomplexed monomeric RPTP β/ζ in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for decreasing the concentration of (PTN)2-(RPTP β/ζ)2 heterotetramer in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for substantially reducing activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing the activity of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing the phosphorylation state of ALK in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for substantially deactivating VEGFR in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing the activity of VEGFR in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

In another aspect, the disclosure provides a method for reducing the phosphorylation state of VEGFR in a cell of a subject comprising administering alone or in combination an effective amount of an antibody against PTN or a fragment thereof, an effective amount of negative PTN or decoy RPTPβ/ζ wherein the cell expresses PTN, RPTP β/ζ and VEGFR and wherein when one or more of an effective amount of antibody against PTN is administered to the subject, an effective amount of negative PTN is administered to the subject, and an effective amount of decoy RPTP β/ζ is administered to the subject, VEGFR is consequently substantially dephosphorylated by RPTP β/ζ.

The disclosure also provides for each of the methods herein that the antibody against PTN or fragment thereof can be a monoclonal antibody, a polyclonal antibody or a humanized monoclonal antibody or a humanized polyclonal antibody.

The disclosure also provides for each of the embodiments herein that the methods comprise administering an effective amount of decoy RPTP β/ζ rather than administering an effective amount of an antibody against PTN or a fragment thereof.

The disclosure also provides for each of the embodiments herein that the methods comprise administering an effective amount of negative PTN rather than administering an effective amount of an antibody against PTN or a fragment thereof.

The disclosure also provides for each of the embodiments herein that the methods comprise administering an effective amount of one or more of an antibody against PTN or a fragment thereof, negative PTN, and decoy RPTP β/ζ or combinations thereof.

### EXAMPLES

### Example 1

### A Novel Oncogenic Mechanism Regulated by ALK Controlling Cell-Cell Adhesion Through “Clashing Phosphates” in the β-Catenin/Cadherin Interface:

Regulation of cell-cell adhesion is a highly dynamic process of fundamental importance needed to maintain the structural integrity of tissues during normal development as well as in the progression of malignant cancers. Example 1 shows the cytokine pleiotrophin (PTN) stimulates phosphorylation of β-catenin tyrosine 333, loss of association of β-catenin with cadherin, and loss of cell-cell adhesion dependent upon activation of Anaplastic Lymphoma Kinase (ALK) through the pleiotrophin/Receptor Protein Tyrosine Phosphatase beta/zeta (RPTPβ/ζ) pathway. The crystal structure of the β-catenin/E-cadherin interface reveals E-cadherin phosphoserine 692 hydrogen bonds with β-catenin tyrosine 333 and establishes charge complementarity with β-catenin lysines 292 and 335. Depending upon whether E-cadherin serine 692, β-catenin tyrosine 333 or both are phosphorylated, cell adhesion either is stabilized or disrupted. The juxtaposition of these alternate phosphorylation sites thus produces a novel molecular switch deemed herein “mutually exclusive reciprocating phosphates” regulated by the pleiotrophin/RPTPβ/ζ pathway. Our finding support a mechanism by which PTN induces a epithelial-mesenchymal transition (EMT) during differentiation, and constitutive expression of PTN in malignant cancers triggers a loss of cell-cell adhesion and induces an EMT-like phenotype.

Adherent junction complexes function to establish reversible interfaces with adjacent cells that are responsive to signals that direct cell movement or cell shape. A loss of epithelial cell-cell junctions and the polarized epithelial phenotype occurs in the process of an epithelial-mesenchymal transition, which, in turn, leads to a more motile fibroblast-like cellular phenotype. Regulation of cell-cell adhesion thus has a central role in embryogenesis, tissue patterning, wound healing, and differentiation (Thiery, J. P. (2002), Nat Rev Cancer 2, 442-454; Thiery, J. P. (2003), Curr Opin Cell Biol 15, 740-746.). Loss of epithelial cell-cell adhesions and the development of an EMT through different mechanisms also is a characteristic hallmark of invasive cancer cells (Thiery, supra).

The present example demonstrates that ALK kinase, activated through the PTN/RPTPβ/ζ signaling pathway, (1) phosphorylates β-catenin tyrosine 333, (2) prevents the association of β-catenin with cadherin, (3) induces loss of cell-cell adhesion through a mechanism we term “mutually exclusive reciprocating phosphates”, and (4) inducing an EMT. We show through analysis of the β-catenin/E-cadherin crystal structure that β-catenin tyrosine 333 occupies a critical position in the interface of β-catenin with cadherin. Our analysis demonstrates phosphoserine 692 in E-cadherin hydrogen bonds with β-catenin tyrosine 333 and establishes charge complementarity with two surrounding tyrosine residues to establish a high affinity association of β-catenin with E-cadherin. Phosphorylation of tyrosine 333 by ALK destroys the interactions of β-catenin tyrosine 333 with cadherin phosphoserine 692 and thus disrupts the high affinity association of β-catenin with cadherin. The data of Example 1 support a novel molecular mechanism through which ALK, activated through the PTN/RPTPβ/ζ signaling pathway, is a significant regulator of cell-cell adhesion and cytoskeletal function. The Example thus identifies a novel pathogenic mechanisms of activated ALK and a new rationale for designing new therapies to target ALK signaling in tumor progression, either directly or indirectly through its interaction with PTN.

The PTN/RPTPβ/ζ pathway and ALK are required for PTN-stimulated tyrosine phosphorylation of β-catenin:

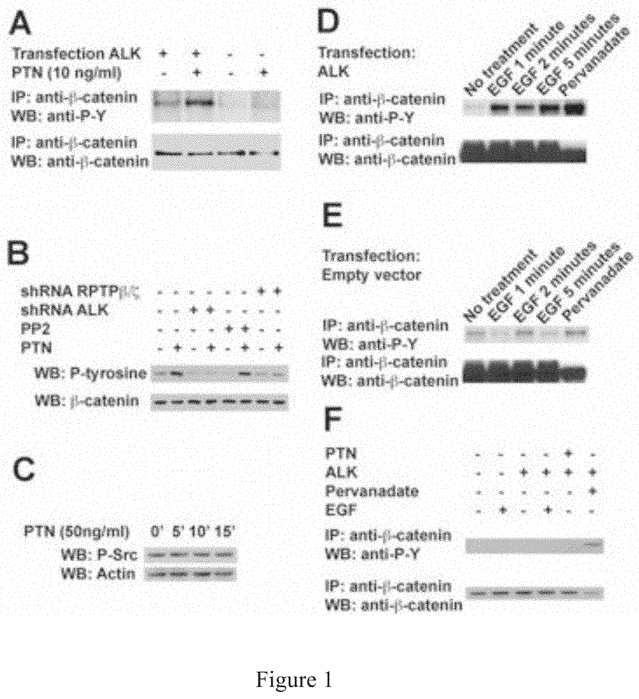

To first indentify the pathway that establishes phosphorylation of β-catenin in PTN-stimulated cells, we transiently transfected lysates from PTN-stimulated COS-7 cells with both RPTPβ/ζ and ALK, immunoprecipitated with anti-β-catenin antibodies, and analyzed the immunoprecipitates in Western blots probed with anti-phosphotyrosine antibodies. We found that PTN stimulates increased tyrosine phosphorylation of β-catenin in COS-7 cells that express both ALK and RPTPβ/ζ by about 3-fold. In contrast, PTN failed to stimulate tyrosine phosphorylation of β-catenin in cells that express RPTPβ/ζ alone as it did in PTN-stimulated COS-7 cells that express both RPTPβ/ζ and ALK (FIG. 1A). Phosphorylation of β-catenin in PTN-stimulated cells thus requires ALK and RPTPβ/ζ.

We then transfected U87MG (human glioblastoma) cells that express endogenous RPTPβ/ζ and ALK with shRNAs to “knock down” either ALK or RPTPβ/ζ; we immunoprecipitated lysates from control and shRNA expressing U87MG cells with anti-β-catenin antibodies and analyzed in Western blots probed with anti-phosphotyrosine antibodies (FIG. 1B). In the control study, phosphorylation of β-catenin was sharply increased in PTN-stimulated cells. In contrast, when either ALK or RPTPβ/ζ were knocked down, PTN failed to stimulate tyrosine phosphorylation of β-catenin (FIG. 1 B). Thus, again, the data indicate both RPTPβ/ζ and ALK are required in the pathway used by PTN to stimulate increased tyrosine phosphorylation of β-catenin (FIG. 1 B).

Since activated Src also is known to phosphorylate β-catenin (Hamaguchi, M., et al., (1993), EMBO J 12, 307-314), the chemical inhibitor of Src activity, PP2, was therefore tested. PP2 failed to inhibit PTN-stimulated tyrosine phosphorylation of β-catenin in U87MG cells (FIG. 1B). Furthermore, when control U87MG cells or U87MG cells stimulated with PTN were lysed and analyzed in Western blots probed with anti-phospho-Src tyrosine 416 antibodies, which specifically recognize activated Src, tyrosine phosphorylation of Src tyrosine 416 was not increased in PTN-stimulated U87MG cells (FIG. 1C). Src activity thus does not stimulate tyrosine phosphorylation of β-catenin in PTN-stimulated cells nor is Src activated by PTN in PTN-stimulated cells.

To verify the requirement for both RPTPβ/ζ and ALK to phosphorylate β-catenin in PTN-stimulated cells, MCF-7 cells that express chimeric epidermal growth factor (EGF) receptor (R)/RPTPβ/ζ were tested. EGFR/RPTPβ/ζ consists of the extracellular domain of EGFR fused with the transmembrane and intracellular domains of RPTPβ/ζ. EGF-stimulated EGFR/RPTPβ/ζ in MCF-7 cells effectively mimics PTN-signaling through the PTN/RPTPβ/ζ signaling pathway (Perez-Pinera, (2007) J Biol Chem 282, 28683-28690). ALK was therefore introduced into MCF-7-EGFR/RPTPβ/ζ cells, the cells were stimulated with EGF, and lysates were prepared. The lysates were immunoprecipitated with anti-β-catenin antibodies and analyzed in Western blots probed with anti-phosphotyrosine antibodies. EGF-stimulated cells increased tyrosine phosphorylation of β-catenin about 11-fold 1 minute after stimulation with EGF. Even higher levels of tyrosine phosphorylation of β-catenin were found at 5 minutes. The apparent lesser increase in tyrosine phosphorylation at 2 minutes was not explained and believed to be an artifact. MCF-7-EGFR/RPTPβ/ζ cells stimulated with the non-specific tyrosine phosphatase inhibitor sodium pervanadate increased tyrosine phosphorylation of β-catenin about 17-fold. (FIG. 1D). In contrast, EGF-stimulated MCF-7-EGFR/RPTPβ/ζ cells that did not express ALK failed to increase tyrosine phosphorylation of β-catenin to the same level, if at all, to that observed in cells that expressed EGFR/RPTPβ/ζ and ALK (FIG. 1E). EGF-stimulated MCF-7 cells that lack the EGFR/RPTPβ/ζ chimeric receptor did not increase tyrosine phosphorylation of β-catenin when transfected with ALK or when stimulated by PTN; but, when stimulated with pervanadate, β-catenin was phosphorylated in tyrosine (FIG. 1F). The data strongly support that the pathway to increase tyrosine phosphorylation of β-catenin in MCF-7-EGFR/RPTPβ/ζ cells stimulated by EGF requires both RPTPβ/ζ and ALK.

### β-Catenin is a Substrate of ALK:

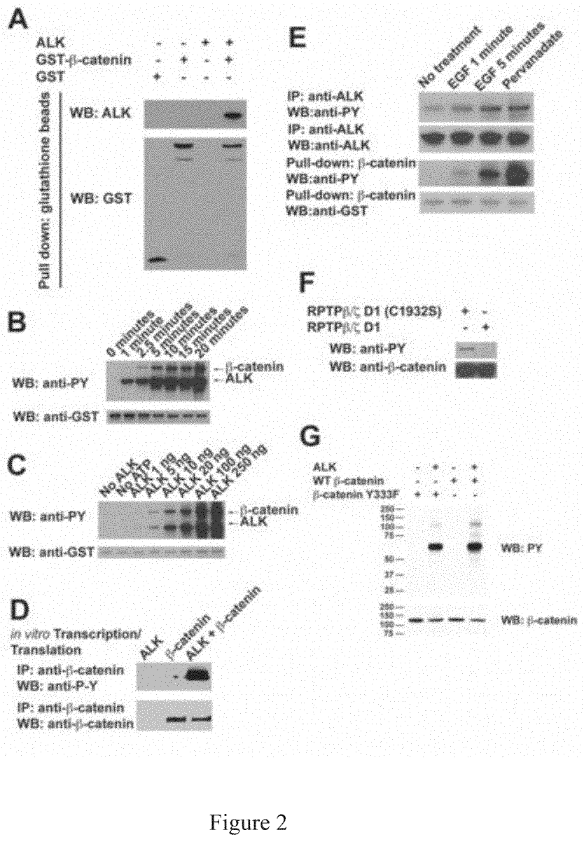

To verify whether β-catenin is a substrate of ALK and that ALK is the kinase that phosphorylates β-catenin in PTN-stimulated cells, recombinant ALK alone, glutathione S-transferase (GST)-β-catenin alone, or ALK plus GST-β-catenin together were incubated with glutathione-Agarose beads. The eluates from the beads were analyzed in Western blots probed with anti-ALK antibodies and probed again with anti-GST antibodies. It was found that only when ALK was incubated together with GST-β-catenin were both ALK and GST-β-catenin identified in Western blots (FIG. 2A), indicating β-catenin and ALK associate together in vitro. To confirm this result in vivo, we show that β-catenin and ALK also associate together in vivo when analyzed with Immunohistochemistry (Supplemental FIG. 2).

ALK auto-phosphorylates through autoactivation in vitro, and auto-phosphorylation of ALK is a marker of activated ALK; GST fused to β-catenin (GST-β-catenin) and ALK were then incubated together with ATP in the in vitro kinase assay previously described (Perez-Pinera et al., 2007, supra). Tyrosine phosphorylation of ALK was seen at 1 minute, and it sharply increased as the time of incubation increased (FIG. 2B). Tyrosine phosphorylation of β-catenin was first detected at 2.5 minutes and sharply increased as the time of incubation with ALK increased. Tyrosine phosphorylation of β-catenin also was ALK concentration dependent (FIG. 2C). These data establish directly that β-catenin is a substrate of ALK. For further confirmation, cDNAs to encode full-length ALK and β-catenin were generated and used for in vitro transcription/translation, incubated together in the in vitro kinase assay and analyzed. β-catenin was phosphorylated only when it was incubated together with ALK in the in vitro kinase reaction (FIG. 2D).

### ALK Phosphorylates β-Catenin in EGF-Stimulated MCF-7-EGFR/RPTPβ/ζ Cells:

The EGF-stimulated MCF-7-EGFR/RPTPβ/ζ cell model described above was then used to activate ALK. Lysates prepared from EGF- or sodium pervanadate (control)-stimulated MCF-7-EGFR/RPTPβ/ζ cells into which ALK had been introduced were immunoprecipitated with anti-ALK antibodies and analyzed in Western blots probed with anti-phosphotyrosine antibodies. A marked increase in tyrosine phosphorylation of ALK was found in MCF-7-EGFR/RPTPβ/ζ cells stimulated with EGF at 1 minute and at 5 minutes (FIG. 2E); thus, ALK is activated under these conditions.

ALK activated in vivo was then immunoprecipitated from lysates of EGF-stimulated MCF-7-EGFR/RPTPβ/ζ cells and tested with β-catenin in the in vitro kinase assay. ALK catalyzed an increase in tyrosine phosphorylation of β-catenin at 1 minute (FIG. 2E), and, at 5 minutes, a striking increase in tyrosine phosphorylation of β-catenin was seen; even higher levels of tyrosine phosphorylation of β-catenin were found when it was incubated with ALK immunoprecipitated from cells stimulated with sodium pervanadate (FIG. 2E). ALK immunoprecipitated with anti-ALK antibodies from MCF-7-EGFR/RPTPβ/ζ cells that had been not stimulated with EGF failed to stimulate tyrosine phosphorylation of β-catenin (FIG. 2E). Again, we show that β-catenin is a substrate of activated ALK.

### The Site in β-Catenin Phosphorylated by ALK is Regulated by RPTPβ/ζ:

Lysates from EGF-stimulated MCF-7-EGFR/RPTPβ/ζ cells that express ALK were then immunoprecipitated with anti-β-catenin antibodies, and the immunoprecipitates were incubated with (1) glutathione S-transferase (GST), fused with the catalytic (active) site D1 domain of RPTPβ/ζ (GST-RPTPβ/ζ D1), or with (2) GST fused with the catalytically inactive site D1 domain of RPTPβ/ζ (GST-RPTPβ/ζ D1 C1932S) and analyzed in Western blots probed with anti-phosphotyrosine antibodies as previously described (Pariser et al., Biochem Biophys Res Commun 335, 232-239). Tyrosine phosphorylation of β-catenin was markedly reduced when β-catenin was incubated with GST-RPTPβ/ζ D1 (FIG. 2F). We predicted that the site in β-catenin phosphorylated by ALK kinase and the site phosphorylated in β-catenin in PTN-stimulated cells previously described (Meng et al., Proc Natl Acad Sci USA 97, 2603-2608) are dephosphorylated by RPTPβ/ζ, may be the same and regulated by RPTPβ/ζ; it was heretofore unknown which specific site that may be and its implications for therapeutic intervention.

### ALK Phosphorylates Tyrosine 333 in β-Catenin:

To then identify the specific residue in β-catenin phosphorylated by activated ALK and in PTN-stimulated cells, we incubated β-catenin with recombinant ALK and ATP in the in vitro kinase assay. The phosphorylated proteins were immunoprecipitated with anti-phosphotyrosine antibodies and the immunoprecipitates were analyzed in SDS polyacrylamide gels stained with Coomassie Blue. The Coomassie Blue stained band corresponding to β-catenin was then eluted and analyzed by mass spectrometry as described in Methods. The residue in β-catenin phosphorylated by ALK was identified as tyrosine 333 (see Supplement FIG. 1).

To confirm these results, we mutated tyrosine 333 in GST-β-catenin to phenylalanine and incubated wild type (wt) GST-β-catenin and GST-β-catenin Y333F with ALK in an in vitro kinase assay. The results confirm ALK phosphorylates β-catenin. However, ALK failed to phosphorylate GST-β-catenin Y333F to the nearly the same extent as wt β-catenin, supporting Tyr333 is the principle tyrosine phosphorylated by ALK in β-catenin and likely the only site in β-catenin phosphorylated by ALK (FIG. 2G).

Phosphorylation of β-Catenin in Tyrosine 333 Activates a Novel Molecular Switch we Term “Clashing Phosphates”, which Ablates Critical Interactions Required for the Association of β-Catenin with Cadherin:

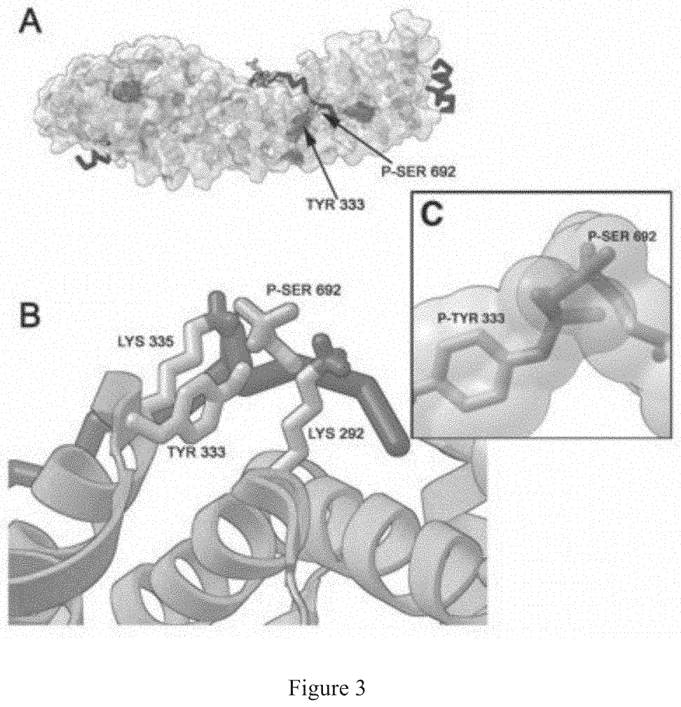

The crystal structure of the β-catenin interface with E-cadherin (Huber and Weis, 2001, Cell 105, 391-402), obtained from the Protein Data Bank file 1I7W (http//www.rcsb.org), was then analyzed. It was seen tyrosine 333 in β-catenin is positioned to potentially have a role in forming the molecular interface of the β-catenin/E-cadherin complex; the other tyrosine residues in β-catenin identified in the model are not positioned to influence the β-catenin/E-cadherin interface through interaction with phosphorylated serines (FIG. 3A). There are two copies of the β-catenin/E-cadherin complex in the crystallographic asymmetric unit. The intermolecular interface of these two complexes is very similar; however, the phosphorylation states of E-cadherin differ at two of the three serines; only serine 692 in E-cadherin is phosphorylated in both independent copies of the molecular complex. Phosphorylation of serine 692 may be more important than the other serines in E-cadherin in the formation of the interface with β-catenin.

The phosphate group of phosphoserine 692 of E-cadherin participates directly in a hydrogen bond with the hydroxyl group of tyrosine 333. It also appears to be involved in charge interactions with lysines 292 and 335 of β-catenin (FIG. 3B). The additional charge complementarity established by phosphoserine 692 in E-cadherin with lysines 292 and 335 of β-catenin likely strengthens the hydrogen bond phosphoserine 692 establishes with β-catenin tyrosine 333. The other serine residues on E-cadherin in the vicinity of this interface do not appear to interact directly with tyrosine residues on the β-catenin.

We surmised that the juxtaposition of the negatively charged phosphates of E-cadherin phosphoserine 692 and β-catenin phosphotyrosine 333 would destroy the β-catenin-cadherin association necessary for cell-cell adhesion; that is, depending upon whether β-catenin tyrosine 333 is or is not phosphorylated, the association of β-catenin with E-cadherin is disrupted or established, respectively. Phosphorylation of tyrosine 333 in β-catenin by activated ALK thus positions this novel molecular switch (FIG. 3C), we term “clashing phosphates”, to disrupt association of β-catenin with E-cadherin and thus to disrupt cell-cell adhesion.

Phosphorylation of β-Catenin in Tyrosine 333 Reduces Association of β-Catenin with Cadherins:

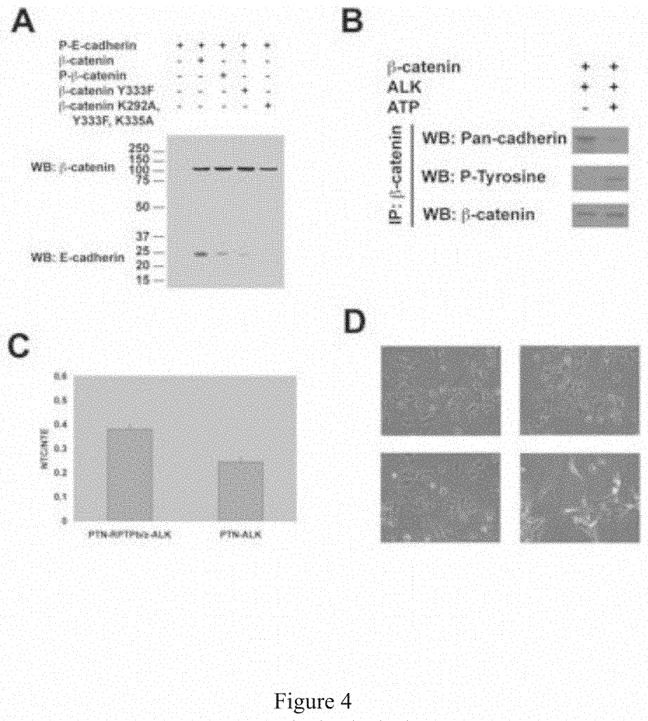

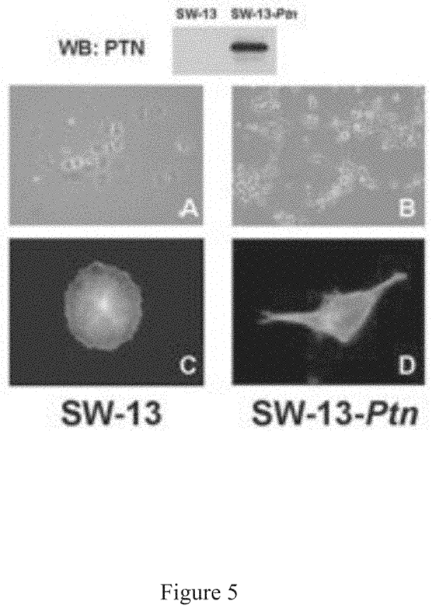

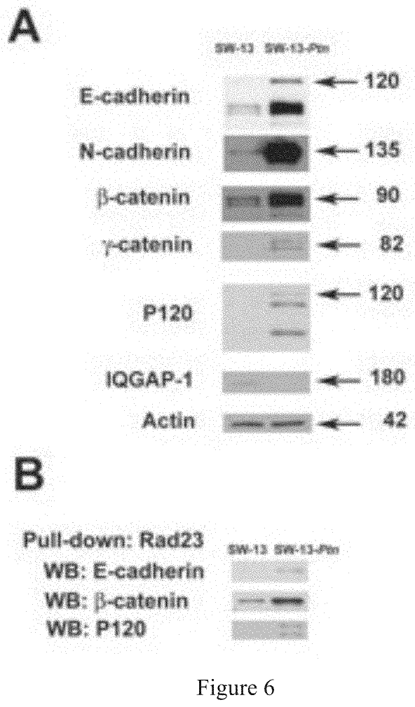

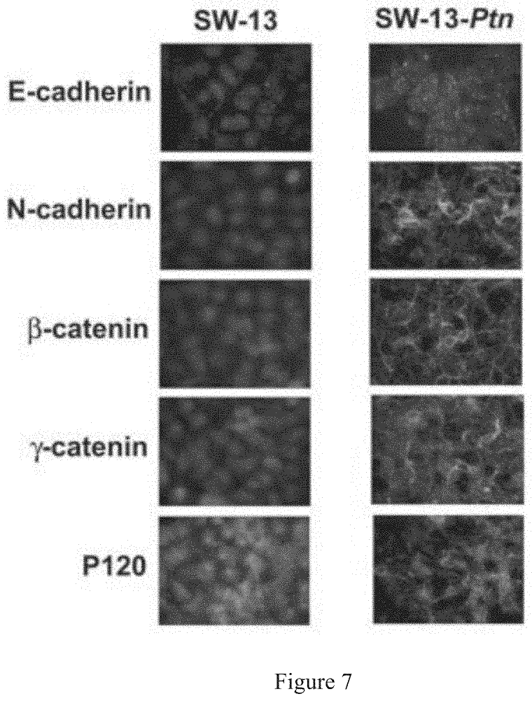

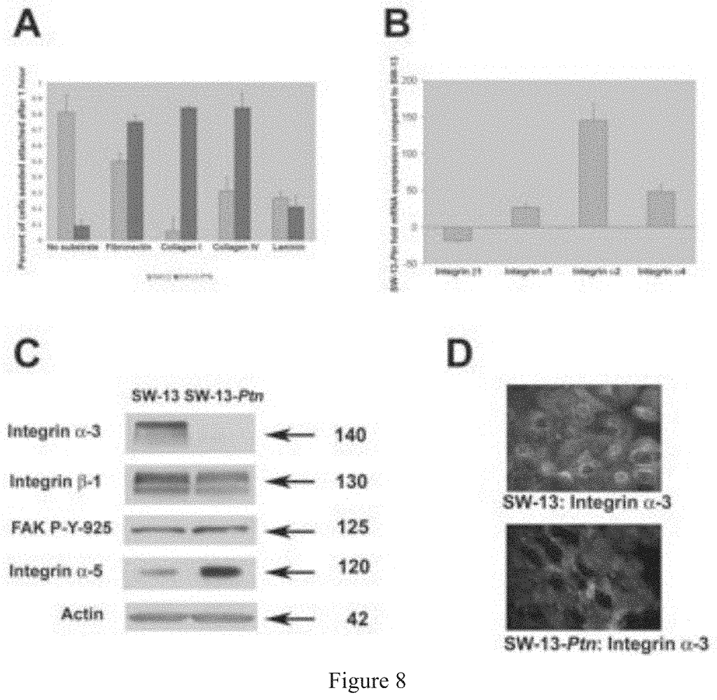

To test our clashing phosphates hypothesis, where the phosphorylation state of β-catenin tyrosine 333 directly affects the association of β-catenin with E-cadherin, we phosphorylated recombinant E-cadherin serine 692 with casein kinase (CK) 2 and Glycogen Synthase Kinase (GSK) 3β as described by Lickert et al. (J Biol Chem 275, 5090-5095) and incubated it with (1) recombinant GST-β-catenin, with (2) GST-β-catenin phosphorylated by ALK in vitro, with (3) GST-β-catenin single mutant Y333F, or with (4) GST-β-catenin triple mutant K292A, Y333F, K335A. The GST-conjugated proteins were captured with glutathione beads and the phospho-Ser692-E-cadherin that associated with the GST-conjugated proteins was eluted and analyzed in Western blots probed with anti-β-catenin and anti-E-cadherin antibodies. In the control, recombinant E-cadherin (non-phosphorylated) did not associate with β-catenin. However, phospho-Ser692-E-cadherin associates readily with non-phosphorylated β-catenin—the levels of phospho-Ser692-E-cadherin that associate with GST-β-catenin phosphorylated in vitro by ALK decreased by about 4.2-fold. The levels of phospho-Ser692-E-cadherin that associated with the GST-β-catenin single mutant Y333F were reduced about 11.6-fold, and the levels of phospho-Ser692-E-cadherin that associated with GST-β-catenin triple mutant K292A, Y333F, K335A were essentially not detectable (FIG. 4A).

These results support the importance of the hydrogen bond between phospho-Ser692-E-cadherin and β-catenin tyrosine 333 in defining the association of E-cadherin and β-catenin. The results further support clashing phosphates is an important mechanism regulating the association β-catenin to E-cadherin in vitro, and they also support the contribution of charge complementarity between E-cadherin phospho-Ser 692 and β-catenin lysines 292 and 335 in the pocket they form with tyrosine 333 in β-catenin, which strengthens the hydrogen bond phosphoserine 692 shares with β-catenin tyrosine 333.

Activated ALK Stimulates Loss of Association of β-Catenin, Loss of Cell-Cell Adhesion and Induces a Morphological Epithelial to Mesenchymal Transition (EMT):

To further test the consequences of the unique molecular switch induced by phosphorylation of tyrosine 333, tyrosine 333 in GST-β-catenin was phosphorylated in vitro by ALK and incubated with lysates of U373 cells to analyze its association with cadherins in cells. GST-β-catenin phosphotyrosine 333 was then captured with glutathione-S-Sepharose and proteins associated with GST-β-catenin phosphotyrosine 333 were analyzed in Western blots probed with anti-cadherin antibodies, anti-β-catenin antibodies, or anti-phosphotyrosine antibodies. The association of cadherin with GST-β-catenin in this assay was sharply lowered when GST-β-catenin was phosphorylated by ALK (FIG. 4B); whereas, in contrast, cadherin readily associated with GST-β-catenin if tyrosine 333 in β-catenin was not phosphorylated by ALK. Thus, phosphorylation of tyrosine 333 in β-catenin by ALK decreases the affinity of the association of β-catenin with cadherin.

Homophilic cell-cell adhesion was then analyzed in MCF-7 cells transfected with RPTPβ/ζ and ALK compared with homophilic cell-cell adhesion of MCF-7 cells transfected with ALK alone, using an assay which measures the ratio of cells dissociated from each other in calcium-containing media (NTC) and the cells dissociated from each other in calcium-free media (NTE) NTC/NTE. The ratio NTC/NTE measured with PTN-stimulated MCF7 cells that express both RPTPβ/ζ and ALK was about 0.38, but the ratio NTC/NTE was about 0.25 in PTN-stimulated MCF-7 cells transfected with ALK alone (FIG. 4C), demonstrating in this assay of homophilic cell-cell adhesion that β-catenin phosphorylated by ALK through the PTN/RPTPβ/ζ signaling pathway significantly reduces homophilic cell-cell adhesion.

Furthermore, we knocked down ALK expression in MDA-MB-231 human breast cancer cells using a specific ALK shRNA and measured calcium-dependent homophilic cell-cell adhesion. The ratio NTC/NTE in MDA-MB-231 cells transfected with an empty vector was about 0.27, whereas the ratio NTC/NTE in MDA-MB-231 cells in which ALK expression was knocked down was about 0.17. Thus, both RPTPβ/ζ, activation of ALK and phosphorylation of tyrosine 333 in β-catenin are required to effectively decrease cell-cell adhesion, which is consistent with the reduced affinity of β-catenin phosphorylated at tyrosine 333 for E-cadherin phosphoserine 692. These results demonstrate that inactivation of ALK in a tumor cell line where ALK is aberrantly activated partially restores cell-cell adhesion.

The EMT is believed to require loss of cell-cell adhesion (Thiery, 2002 supra); in our earlier studies (Perez-Pinera et al., 2006, Proc Natl Acad Sci USA 103, 17795-17800) it was found that PTN stimulated an EMT with loss of cadherin function due to its ubiquitination and degradation. However, whether loss of cell-cell adhesion stimulated through activation of the PTN/RPTPβ/ζ signaling pathway is sufficient to induce an EMT was not known. We tested the potential that ALK activation and phosphorylation of β-catenin tyrosine 333 are sufficient to induce an EMT. MCF10A cells were transfected with both RPTPβ/ζ and ALK and stimulated with PTN as above; the cells became more elongated, developed two or more filopodia, and assumed a mesenchymal fibroblast-like shape characteristic of the motile, invasive cells undergoing an EMT. In contrast, the MCF-10A cells transfected with a control vector that lacked ALK effectively established cell-cell adhesion and retained the flat, round morphological appearance of epithelial cells (FIG. 4D). These data support the conclusion that ALK, activated through the PTN/RPTPβ/ζ signaling pathway, and phosphorylation of β-catenin tyrosine 333 have an essential role and potentially may be sufficient in the induction an EMT in PTN-stimulated cells.

We transfected MDA-MB-231 breast cancer epithelial cells, which express ALK activated through the PTN/RPTPβ/ζ signaling pathway, with an ALK-specific shRNA or with an empty vector and measured the ratio NTC/NTE. The ratio NTC/NTE in MDA-MB-231 cells transfected with an empty vector was about 0.27; whereas, the ratio NTC/NTE in MDA-MB-231 cells in which ALK expression was knocked down was about 0.17. The studies thus indicate in MDA-MB-231 cells that ALK, activated through the PTN/RPTPβ/ζ signaling pathway, and phosphorylation of tyrosine 333 in β-catenin are required to effectively decrease cell-cell adhesion in these malignant cells. This is consistent with the reduced affinity of β-catenin, when phosphorylated at tyrosine 333, for E-cadherin phosphoserine 692, reduced association of β-catenin with E-cadherin and reduced cell-cell adhesion.

### Discussion

Loss of cell-cell adhesion is a required step in the reversible process of epithelial-mesenchymal transitions needed for cells to migrate during normal development and for the malignant cancer cells to advance to the more motile, fibroblast-like phenotype characteristic of highly malignant cancer cell (Thiery 2002, supra). Dissecting the molecular mechanisms that regulate cell-cell adhesions is thus of fundamental importance to better appreciate the critical transitions needed for migration and differentiation in normal development and their roles in the pathogenesis of malignant cell progression to higher states of malignancy. We believe we have done that in the present Example.

In the present Example, a new pathway that regulates cell-cell adhesion has been identified; the Example data indicate cell-cell adhesion is regulated through phosphorylation of β-catenin tyrosine 333 by ALK, which itself is activated through an alternative mechanism of activation of receptor tyrosine kinases induced by PTN through the PTN/RPTPβ/ζ signaling pathway (Perez-Pinera et al., 2007, supra). The data of this Example support the conclusion that phosphorylation of tyrosine 333 of β-catenin disrupts the bond with E-cadherin serine 692 and thus β-catenin tyrosine 333 is a novel molecular switch based upon “mutually exclusive reciprocating phosphates” between β-catenin tyrosine 333 and cadherin 692. The data in the present Example support the hypothesis that this previously unknown switch is a significant mechanism regulating cell-cell adhesion. Depending upon whether E-cadherin serine 692 or β-catenin tyrosine 333 are phosphorylated, cell-cell adhesion is established or disrupted. The data also support the conclusion that PTN, through regulating the PTN/RPTPβ/ζ signaling pathway and thus controlling the levels of activation of ALK, is an important determinant that establishes the balance of phosphorylation levels of β-catenin tyrosine 333. Through regulation of phosphorylation of β-catenin tyrosine 333, PTN functions to regulate the molecular switch that regulates cell-cell adhesion. This mechanism, which we have termed “mutually exclusive reciprocating phosphates”, has not been previously reported and provides a powerful means of regulating β-catenin phosphorylation state and cell-cell adhesion. Phosphorylation of β-catenin tyrosine 333 by ALK alone appears sufficient to be a significant mechanism that induces malignant cells to assume the aggressive invasive phenotype characteristic of the EMT. Thus the instant Example indicates that activation of this new pathway incrementally stimulates tumor progression and is a significant contributor to the oncogenic impact of ALK in perhaps many malignant cancers, and that targeting ALK or other members of the pathway, like PTN, may reverse or prevent cellular EMT, slow, halt or reverse cancer progression. In other words, the present Example describes a novel pathway and unique new mechanism through which ALK activated through the PTN/RPTPβ/ζ signaling pathway in malignant cells may be oncogenic. We demonstrate that activated ALK phosphorylates β-catenin, a previously unknown substrate of ALK, and that the site in β-catenin phosphorylated by ALK is tyrosine 333, which effectively disrupts the association of β-catenin with E-cadherin. The result is a loss of cell-cell association and a morphological transition of cells to an invasive, mesenchymal phenotype. The Example thus describes a novel substrate of ALK and both a unique oncogenic pathway and new mechanism for ALK to stimulate the EMT and tumor progression through the PTN/RPTPβ/ζ signaling pathway.

### Experimental Procedures

### E-Cadherin Nomenclature:

Mouse E-cadherin (GeneID12550, NM_009864.2) was used by Huber and Weiss (Cell. 2001 May 4; 105(3):391-402) to determine the crystal structure of the β-catenin/phosphorylated E-cadherin complex (PDB 1I7W), which was used to discover the consequences of phosphorylation of tyrosine 333 in β-catenin in β-catenin/E-cadherin association. The phosphorylated serine that interacts with tyrosine 333 in PDB 1I7W is serine 692, which corresponds to Ser848 in other databases.

### ALK Phosphorylated in PTN-Stimulated Cells is Dephosphorylated by RPTPβ/ζ:

Three 100 mm culture dishes of MCF7 cells that express Epidermal Growth Factor (EGF) Receptor (R) (EGFR)/RPTPβ/ζ were grown to about 70% confluence in DMEM media with 10% FBS, serum starved for 24 hours, and stimulated with EGF at 150 ng/ml (R&D Systems, Minneapolis, Minn.) for 1 minute or with sodium pervanadate at 20 μg/ml for about 20 minutes. Cell lysates were prepared and immunoprecipitated with anti-ALK antibodies. The immunoprecipitates were incubated with GST-RPTPβ/ζ D1 or GST-RPTP6/D1 (C19325) as previously described (Pariser et al., 2005, Biochem Biophys Res Commun 335, 232-239). The D1 (domain) is the cytoplasmic domain that contains the active site of RPTPβ/ζ. GST-RPTPβ/ζ D1 is an N-terminal GST-coupled to the RPTPβ/ζ D1 domain; GST-RPTPβ/ζ D1 (C19325) is an N-terminal GST-coupled to the active site-inactivated D1 domain mutant. After incubation, the levels of tyrosine phosphorylation of β-catenin were then analyzed by scanning densitometry of Western blots probed with anti-phosphotyrosine antibodies.

### Kinase Assays:

ALK alone, or together with one microgram of β-catenin, was incubated with 1 mM ATP and a buffer containing 25 mM Tris-HCl (pH 7.5), 5 mM β-glycerophosphate, 2 mM dithiothreitol (DTT), 0.1 mM Na3VO4, 10 mM MgCl2 for at 37° C. The samples were analyzed in Western blots probed with anti-phosphotyrosine antibodies and reprobed with anti-GST antibodies or anti-ALK antibodies as appropriate. As negative control, samples without ALK or without ATP were analyzed.

### Molecular Structure Analysis:

The structure analysis of the β-catenin interface with E-cadherin was carried out on Protein Data Bank file 1I7W (http://rcsb.org), which contains two copies of the complex in the crystallographic asymmetric unit. The Python Molecular Viewer (PMV) (Sanner, 2005, Structure 13, 447-462) was used for interactive visualization, measurement, and visual analysis of the molecular complex.

### Example 2

### Pleiotrophin Regulates Expression of Key Genes in Development and Tumor Progression Through Activation of Epidermal Growth Factor Receptor and Nuclear Import of β-Catenin

Pleiotrophin (PTN, Ptn) is a developmentally regulated cytokine with key roles in differentiation during late embryogenesis and in progression to more malignant phenotypes. The cytokine pleiotrophin inactivates the catalytic activity of its receptor, the receptor protein tyrosine phosphatase (RPTP)β/ζ, which interacts with key cytoplasmic proteins and transmembrane receptor tyrosine kinases to regulate steady state levels of tyrosine phosphorylation. Previous studies have demonstrated PTN regulates tyrosine phosphorylation of β-catenin in PTN-stimulated cells, mediates loss of cell-cell adhesion and a more motile cellular phenotype. We now demonstrate PTN stimulates import of β-catenin into nuclei in PTN-stimulated cells through activation of the epidermal growth factor receptor and the alternative mechanism of receptor tyrosine kinase activation. It is furthermore demonstrated levels of β-catenin in nuclei in malignant cells with high level expression of Ptn. It is shown β-catenin in nuclei associates with members of the Tcf/Lef family and genes known to be regulated by the Tcf/Lef family are differentially transcribed in PTN-stimulated cells. The data indicate PTN coordinately regulates key proteins in different critical cellular systems through the PTN/RPTPβ/ζ signaling pathway to now include transcription activation of diverse signaling pathways critical during embryonic development and in transformed cell growth.

Pleiotrophin (PTN, Ptn) is an 18 kDa heparin binding cytokine that shares about 50% amino acid sequence identity and significant functional overlap with midkine (MK, Mk), the only members of this small family of developmentally regulated cytokines (Milner, Li et al., 1989, Biochem Biophys Res Commun 165(3): 1096-103; Herradon, Ezquerra et al., 2005, Biochem Biophys Res Commun 333(3): 714-21). Pleiotrophin signals through the Receptor Protein Tyrosine Phosphatase (RPTP)β/ζ (Meng, Rodriguez-Pena et al., 2000, Proc Natl Acad Sci USA 97(6): 2603-8); it induces a conformational change in RPTPβ/ζ that inactivates its catalytic activity, permitting activated tyrosine kinases to increase tyrosine phosphorylation of the downstream targets of the PTN/RPTPβ/ζ signaling pathway at sites otherwise dephosphorylated by RPTPβ/ζ (Meng, Rodriguez-Pena et al. 2000, supra).

The first target of the PTN/RPTPβ/ζ signaling pathway was β-catenin (Meng, Rodriguez-Pena et al. 2000, supra). Recently, we found that Anaplastic Lymphoma Kinase (ALK) is activated through the PTN/RPTPβ/ζ signaling pathway (Perez-Pinera, Zhang et al., 2007, supra), through a unique new alternative mechanism to activate receptor tyrosine kinases (RTKs) that disrupted the normal balance of autoactivation and autophosphorylation of ALK maintained by RPTPβ/ζ independent of a direct interaction of PTN with ALK. Our finding was important, as it uncovered a possible mechanism to maintain steady state levels of ALK activation. We hypothesized that perhaps other RTKs may be regulated through the PTN/RPTPβ/ζ signaling pathway as well. Our discovery was also important since we then showed ALK, activated through the PTN/RPTPβ/ζ signaling pathway, is the kinase that phosphorylates β-catenin in PTN-stimulated cells, leading to loss of association of β-catenin with cadherin and cell-cell adhesion, and to induction of an EMT.

Our previous studies demonstrated PTN phosphorylates β-catenin in PTN-stimulated cells (Meng et al. 2000, Proc Natl Acad Sci USA, March 14; 97(6):2603-8). Subsequent studies confirmed phosphorylation of β-catenin in PTN-stimulated cells and furthermore demonstrated β-catenin no longer associates with cadherin; there was loss of cell-cell adhesion and the cells underwent an epithelial-mesenchymal transition (EMT) with loss of E-cadherin function through degradation through the ubiquitin proteasome proteolytic pathway (Perez-Pinera et al 2006, supra). The loss of β-catenin association with cadherin raised the possibility the β-catenin pool in cytoplasm may increase and potentially translocate to nuclei.

We show in the present example that β-catenin is translocated to the nucleus in PTN-stimulated cells through a pathway that shares features with translocation of β-catenin into nucleus in Wnt stimulated cells. β-catenin associates with members of the TCF/LEF transcription factor family in nuclei and, in PTN-stimulated cells, induces a transcription profile of genes some of which are known to be activated during embryonic development, wound healing, tumor progression, and regulation of the cell cycle.

### Experimental Procedures:

### Cell Lines:

The human umbilical vein stromal cell line (American Type Culture Collection, Manassas, Va.) was grown in Ham's F12 supplemented with 0.1 mg/ml heparin, 0.03 mg/ml endothelial cell growth supplement, 10% FBS and 1% penicillin/streptomycin. We designated SW-13 cells (American Type Culture Collection, Manassas, Va.) expressing the “empty” vector as “SW-13” and SW-13 cells expressing the full length Ptn (GenBank accession number NM_002825) as “SW-13-Ptn” cells. The SW-13 cell line is derived from a slow growing adrenal adenocarcinoma and was grown in DMEM supplemented with 10% FBS and 1% penicillin/streptomycin. Both cell lines were maintained in a 37° C. atmosphere with 5% CO2.

### Plasmids and Transfections:

Human full-length ALK cDNA, human full-length RPTPβ/ζ cDNA, and truncated ALK cDNA to encode the 8 membrane proximal extracellular amino acids and the intact transmembrane and intracellular domain of ALK (amino acids 1027 to 1620) with the IgGκ signal peptide sequences were inserted in the pcDNA3.1 expression vector. Transfections were performed using the Fugene 6 transfection reagent (Roche Diagnostics, Alameda, Calif.) following manufacturer's instructions.

Vectors containing shRNA targeted RPTPβ/ζ and a control vector were obtained from Open Biosystems (Huntsville, Ala.) and delivered to the cells using Arrest-In transfection reagent following manufacturer's recommendations. Ninety-six hours after transfection, the levels of expression of RPTPβ/ζ were analyzed using Western blots and real time RT-PCR. The vector pC4-Fv1 E was a kind gift from ARIAD Pharmaceuticals, Inc. (Cambridge, Mass.). The cDNA sequence encoding the intracellular domains of RPTPβ/ζ was inserted in frame with the Fv fragment of FKBP12.

RPTPβ/ζ D1 and D1 (C1932S) GST “Capture” of ALK from Cell Lysates:

Proteins interactive with GST-RPTPβ/ζ D1, GST-RPTPβ/ζ D1 (C1932S), and GST-RPTPβ/ζ D1 (D1900A) were captured as described before (Pariser, Perez-Pinera et al. 2005) and analyzed in Western blots probed with anti-ALK antibodies and separately with anti-GST antibodies.

### Antibodies:

Anti-EGFR antibodies and anti-phosphotyrosine antibodies were obtained from Upstate (Waltham, Mass.), anti-RPTPβ/ζ antibodies were obtained from R&D Systems (Minneapolis, Minn.), anti-β-catenin, anti-γ-catenin, anti-P120, and anti-Orc2 antibodies were obtained from BD Biosciences, San Diego, Calif. Anti-phosphoserine 473 Akt, anti-phosphothreonine 308 Akt, anti-phosphoserine 9 GSK3β, and anti-phosphoserine 33, 37 and threonine 41 β-catenin antibodies were obtained from Cell Signaling Technology (Beverly, Mass.). Anti-Tcf1 and anti-Tcf4 antibodies were obtained from Exalpha, Watertown, Mass. Anti-Tcf2/Lef antibodies were obtained from Upstate, Waltham, Mass. Anti-mouse IgG FITC-conjugated and anti-actin antibodies were obtained from Sigma-Aldrich Co. Ltd, Dorset, UK. Anti-ubiquitin antibodies were obtained from Chemicon, Temecula, Calif. Anti-mouse IgG HRP-conjugated and anti-rabbit IgG HRP-conjugated antibodies were obtained from Santa Cruz Biotechnology, Santa Cruz, Calif.

### Isolation of Nuclei:

Cells at confluence were washed with PBS ×2 and incubated for 10 minutes in a lysis buffer containing 100 mM HEPES pH 7.9, 100 mM KCl, 100 mM EDTA, 1 mM DTT, Complete EDTA-free Protease Inhibitor Cocktail (Roche, Indianapolis, Ind.) and 0.04% IGEPAL (Sigma-Aldrich Co. Ltd, Poole, Dorset, UK). The cells were harvested and centrifuged to pellet the nuclear fractions that were lysed by incubation in a buffer containing 100 mM HEPES, 2 M NaCl, 5 mM EDTA, 50% glycerol, Complete EDTA-free Protease Inhibitor Cocktail and 10 mM DTT. Samples were centrifuged and the supernatant nuclear extract was collected and analyzed in Western-blots as described below. Orc-2, a protein that controls DNA replication, was used as a marker of the nuclear fraction. It was detected with anti-Orc2 antibodies; tubulin was used to mark cytosolic fractions and detected with anti-tubulin antibodies. Samples of the nuclear fractions were used only if tubulin was not detected.

### Immunoprecipitation:

Cell lysates prepared in a lysis buffer containing 50 mM Tris, 150 mM NaCl pH 7.5, 1% NP40, 0.25% sodium deoxycholate, 0.1% SDS, Complete EDTA-free Protease Inhibitor Cocktail (Roche, Indianapolis, Ind.), and 2 mM sodium orthovanadate were pre-cleared by incubation with 2 μg antibodies followed by one hour incubation with protein G-agarose beads. The beads were washed 4 times in the same lysis buffer, eluted in loading buffer containing 240 mM Tris pH 6.8, 40% glycerol, 20% SDS, 2.5% β-mercaptoethanol and 0.01% bromophenol blue and analyzed in Western blots probed with different antibodies as individually described.

### Akt Kinase Assay:

Akt kinase assays were performed using the Akt Kinase Assay Kit obtained from Cell Signaling Technology, Beverly, MA, following manufacturer's recommendations. Cells were harvested under non-denaturing conditions with the lysis buffer provided and lysates were used to immunoprecipitate Akt with anti-Akt specific antibodies. The immunoprecipitate was incubated with kinase buffer, containing 10 μM ATP, and 1 μg of GSK3β fusion protein for 30 minutes. The reaction was terminated by addition of loading buffer and samples were analyzed in Western-blots.

### Ubiquitination Assay:

Human umbilical vein stromal cells were lysed in a buffer prepared with 50 mM Tris, pH 7.2, 150 mM NaCl pH 7.5, 1% NP40, 0.25% sodium deoxicholate, Complete EDTA-free Protease Inhibitor Cocktail and 2 mM sodium orthovanadate. The cell lysates were incubated overnight with 50 μl of agarose beads conjugated with Rad23 (Calbiochem, La Jolla. Calif.), a protein that binds specifically to poly-ubiquitinated proteins (Chen and Madura 2002). The beads were washed 4 times with lysis buffer and analyzed using Western blots.

### Western-Blots:

Samples were loaded on to polyacrylamide gels ranging from 8-15% as appropriate. The resulting gels were transferred to nitrocellulose membranes that were blocked with 50 mM Tris, 150 mM NaCl, 0.1% Tween-20 (TBS-T) and 5% non-fat milk for 1 hour and incubated with primary antibodies in TBS-T supplemented with 5% bovine serum albumin. The membranes were washed three times in TBS-T, incubated for one hour with donkey anti-mouse secondary antibodies conjugated with horseradish peroxidase in TBS-T with 5% non-fat milk, washed 3 times in TBS-T. The immunoreactive proteins were visualized using the ECL Enhanced Method (Amersham, San Francisco, Calif.).

### Immunofluorescence Microscopy:

Cells grown on coverslips were fixed using 4% paraformaldehyde in 0.12 M phosphate buffer pH 7.2 for 30 minutes and washed three times with PBS. Non-specific antibody binding was reduced by incubation in PBS with 1% BSA. The cells were permeabilized using 0.5% Triton X-100 in PBS for 1 hour at room temperature, incubated overnight with the primary antibodies in PBS with 1% BSA and 0.5% Triton X-100, washed three times for 5 minutes with PBS, and incubated in the secondary antibodies conjugated with fluorescein diluted in PBS with 1% BSA and 0.5% Triton X-100. The slides were washed in PBS, mounted using ProLong Antifade Kit (Molecular Probes, Eugene, Oreg.) according to manufacturer's instructions and observed in a Nikon Eclipse E600 microscope.

### Luciferase Reporter Assay:

SW-13 and SW-13-Ptn cells were grown in 96 well plates. Fugene 6 Transfection Reagent (Roche, Indianapolis, Ind.) was used to deliver Top-Flash or Fop-flash plasmids into the cells (Upstate, Waltham, Mass.) together with the pSV-β-gal vector containing the DNA sequence encoding β-galactosidase. The plasmids were obtained from Promega (Madison, Mich.). Cell lysates were used to measure luciferase activity using the Luciferase Reporter Gene Assay constant light signal (Roche, Indianapolis, Ind.) and to measure β-galactosidase activity using the β-Gal Reporter Gene Assay (Roche, Indianapolis, Ind.), according to manufacturer's instructions. Luciferase activity was determined using a Trilux luminescence counter (PerkinElmer, Wellesley, Mass.). The results were expressed as Relative Luciferase Units after subtraction of the Fop-flash background luminescence signal and normalized according to β-galactosidase activity that was measured using a pQuant spectrophotometer (Biotek, Winooski, Vt.).

### Quantitative Real Time RT-PCR:

RNA from SW-13 and SW-13-Ptn cells was extracted using the RNeasy Mini kit (Qiagen, Valencia, Calif., USA), following the manufacturer's recommendations. The integrity of RNA was checked using agarose gel electrophoresis. Preparations were treated with DNase obtained from Ambion (Austin, Tex., USA) and reverse transcription performed using the SuperScript First Strand Synthesis System (Invitrogen, La Jolla, Calif., USA) with random hexamers. The cDNA was treated with Ribonuclease A (Invitrogen, Carlsbad, Calif.) and RT-PCR amplification was performed in a 50 μL reaction containing SYBR® Green I, 30 U/ml Platinum® Taq DNA polymerase, 20 mM Tris-HCl, pH 8.4, 50 mM KCl, 0.2 mM dNTP mixture, 1.5 mM MgCl2, 0.2 μM primer forward, 0.2 μM primer reverse and 2 μl of cDNA from the previous reaction. Samples were analyzed using the iCycler iQ™ Real Time PCR detection system (BioRad, Hercules, Calif.).

The results presented are the average with standard deviation of three independent experiments using triplicates of each sample relative to the expression of the “housekeeping” gene cyclophilin A.

### Results

Pleiotrophin Stimulates β-Catenin Import into Nuclei:

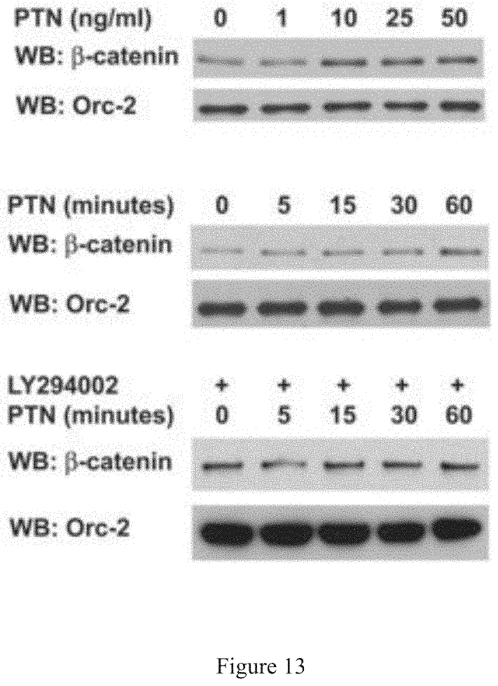

To test the possibility β-catenin is translocated into nuclei of PTN-stimulated HUVS cells, nuclear extracts prepared from PTN-stimulated and non-stimulated HUVS cells were analyzed in Western blots probed with anti-β-catenin antibodies (FIG. 13); we found that PTN stimulated a PTN concentration and time-dependent increase in β-catenin into nuclei of HUVS cells (FIG. 13A). The increase in β-catenin in nuclei of PTN-stimulated cells was seen at 10 ng/ml and more so at 25 and 50 ng/ml PTN. The increase was rapid; it was seen within five minutes of stimulation and continued to increase over the 60 minutes of the experiment. The instant Example thus establish β-catenin is rapidly translocated into nuclei of HUVS cells.

Low levels of β-catenin were readily identified in nuclei of HUVS cells that were not stimulated with PTN, consistent with the possibility that low level expression of endogenous PTN may stimulate low level import of β-catenin needed for steady-state functions of critical transcription factors known to be regulated through nuclear β-catenin.

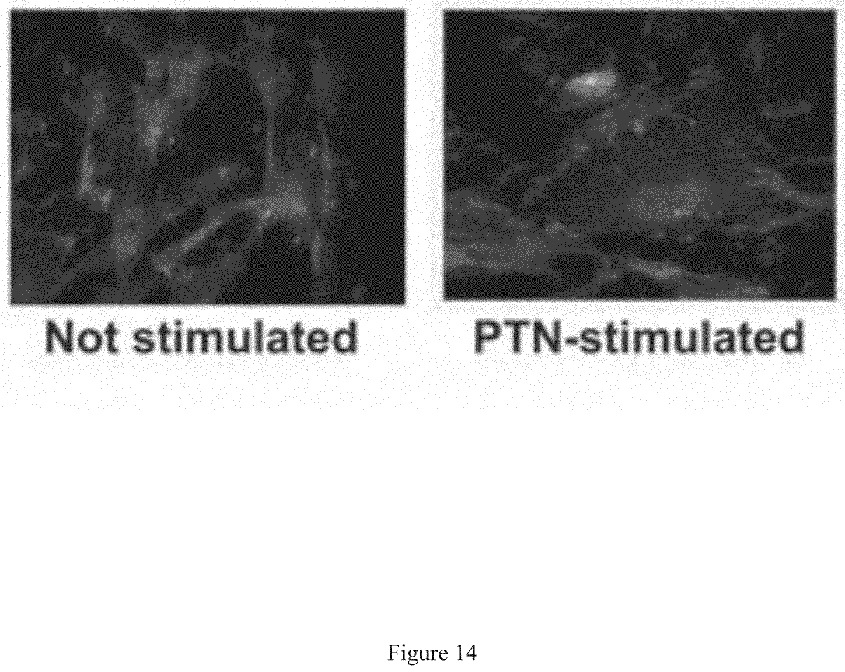

HUVS cells stained using anti-β-catenin antibodies were then analyzed with confocal microscopy (FIG. 14). In HUVS cells not treated with PTN, β-catenin was localized in adherent junction complexes at sites of cell-cell contact. In contrast, in HUVS cells stimulated with PTN, β-catenin was identified in perinuclear regions and in nuclei of many HUVS cells (FIG. 13B), confirming β-catenin is translocated into nuclei of PTN-stimulated cells.

### Ubiquitination of β-Catenin is Decreased in PTN-Stimulated Cells:

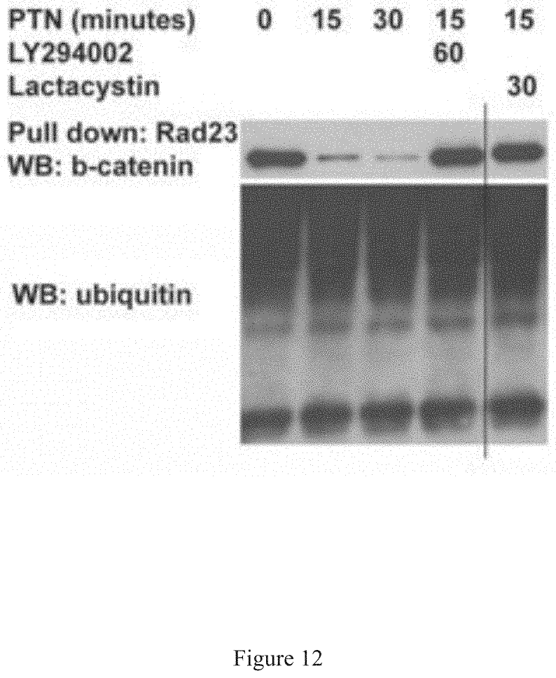

β-catenin is rapidly degraded through the ubiquitin proteasome proteolytic pathway, thereby setting steady state levels of β-catenin by balancing its degradation with new synthesis of this key regulatory protein. This regulatory system is disrupted when the Wnt signaling pathway is activated; that is, β-catenin no longer is substantially ubiquitinated and degraded. As a consequence, the cytoplasmic pool of β-catenin is increased, leading to import of β-catenin into nuclei of Wtn-stimulated cells. We hypothesized that PTN would disrupt ubiquitination of β-catenin, so lysates from PTN-stimulated cells were incubated with Rad23, a protein known to bind ubiquitinated proteins, coupled with Sepharose. Proteins captured with Rad23 Sepharose were eluted and analyzed in Western blots probed with anti-β-catenin antibodies (FIG. 12, upper panel) and the blots were reprobed with anti-ubiquitin antibodies (FIG. 12, lower panel). We found that ubiquitinated β-catenin decreased sharply 15 and 30 minutes after the cells were stimulated with PTN. At 60 minutes, levels of ubiquitinated β-catenin had returned to levels of non-stimulated cells. In the control study, when PTN-stimulated cells were pre-incubated with lactacystin to inhibit proteasome proteolytic activity, ubiquitinated β-catenin levels remained at the level in HUVS cells not stimulated with PTN (FIG. 12, lanes 7 and 8).

Pleiotrophin thus effectively blocks ubiquitination of β-catenin in PTN stimulated cells. The effect is transient; ubiquitinated β-catenin returned to levels of non-treated cells within one hour. Our data support PTN, like Wnt, effectively blocks ubiquitination and presumably degradation of β-catenin in PTN-stimulated cells; β-catenin may be imported into nuclei of PTN-stimulated cells into through mechanisms similar to those used in Wnt-stimulated cells. Pleiotrophin stimulates import of β-catenin into nuclei of PTN-stimulated cells through mechanisms previously described in Wnt-stimulated cells:

Activated Glycogen Synthase Kinase (GSK)3β phosphorylates β-catenin thus enabling β-catenin ubiquitination and the initiation of its degradation through the ubiquitin proteasome proteolysis pathway. In the canonical Wnt pathway, GSK3β is inactivated by Akt (and activated by phosphatidyl inositol 3-kinase (PI3K)), thereby preventing ubiquitination of β-catenin, stabilizing cytosolic β-catenin, and initiating its nuclear import.

PI3K is a downstream target of the PTN/RPTPβ/ζ signaling pathway. To resolve the possibility PTN may stabilize β-catenin through PI3K in PTN-stimulated cells, we measured the levels of the ubiquitinated β-catenin in lysates from PTN-stimulated HUVS cells pretreated for 30 minutes with LY294002, an inhibitor of PI3K; it was found levels of ubiquitinated β-catenin were sharply reduced (FIG. 12, lanes 5, 6), indicating PI3K is required for PTN to effectively block ubiquitination of β-catenin in PTN-stimulated cells.

Pleiotrophin Activates EGFR through Inactivation of RPTPβ/ζ:

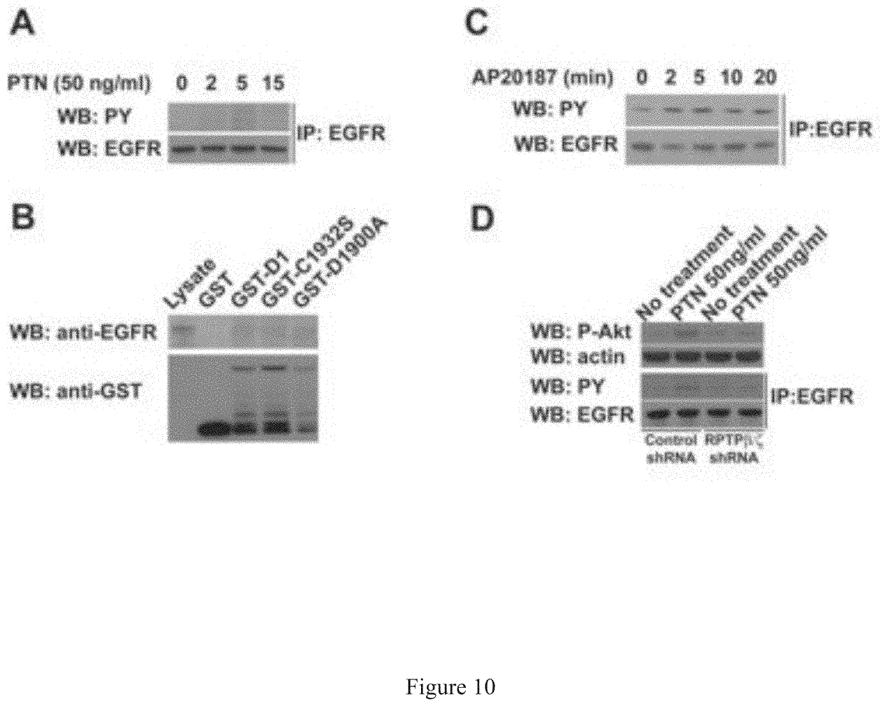

PI3K also is activated by an activated Epidermal Growth Factor Receptor (EGFR). We therefore investigated the possibility EGFR is implicated in the translocation of β-catenin to nucleus in PTN-stimulated cells. HUVS were stimulated with PTN for 2, 5, and 15 minutes. Lysates from these cells were immunoprecipitated with anti-EGFR antibodies and the immunoprecipitates were analyzed in Western blots probed with anti-phosphotyrosine antibodies. Tyrosine phosphorylation of EGFR was increased about 5.8-fold at two minutes after HUVS cells were stimulated with PTN, about 11-fold at 5 minutes, and about 5-fold 15 minutes after stimulation with PTN (FIG. 10A). The data demonstrate that EGFR is rapidly phosphorylated by PTN in PTN-stimulate cells; phosphorylation of EGFR is maximal at 5 minutes, and, it appears to be reduced subsequently.

To establish whether phosphorylation of EGFR is dependent on enforced dimerization of RPTPβ/ζ, which is the consequence of the interaction of PTN with RPTPβ/ζ, HUVS cells were then co-transfected with a full-length EGFR and the vector pC4-Fv1 E. The vector pC4-Fv1 E encodes the Fv domain of FKBP12 in frame with the intracellular domain of RPTPβ/ζ; it effectively enforces dimerization of RPTPβ/ζ when cells are stimulated with AP20187. AP20187 (2 μM) was added to HUVS cells that expressed full-length EGFR and pC4-Fv1 E as above.

AP20187 stimulated increases in tyrosine phosphorylation of EGFR about 4-fold at 2 minutes, about 3-fold at 5 minutes, about 2-fold at 10 minutes, and about 3-fold at 20 minutes after the addition of AP20187 (FIG. 10C), supporting enforced dimerization of RPTPβ/ζ either stimulated by PTN or through the chemically enforced dimerization of its intracellular domain alone sufficient to stimulate increased tyrosine phosphorylation of EGFR. To further confirm this conclusion, a retroviral vector (pSM2) encoding an shRNA to “knock down” RPTPβ/ζ was tested in HUVS cells that express endogenous RPTPβ/ζ and EGFR; in the control experiments, PTN stimulated about a 5-fold increase in the levels of tyrosine phosphorylation of EGFR in cells transfected with a pSM2 control vector. In contrast, in HUVS cells in which RPTPβ/ζ was “knocked down” with the pSM2 vector containing the RPTPβ/ζ shRNA, PTN failed to stimulate increased tyrosine phosphorylation of EGFR. Our data thus indicate PTN stimulates tyrosine phosphorylation of EGFR through the PTN/RPTPβ/ζ signaling pathway (FIG. 10D). The data furthermore indicate PTN regulates steady state levels of tyrosine phosphorylation of EGFR through regulation of the activity of RPTPβ/ζ.

The “substrate trap” mutant captures the substrate phosphoryl-intermediate in the active site of RPTPβ/ζ with high affinity and specificity (Dewang, Hsu et al. 2005, Curr Med Chem 12(1): 1-22). To test the potential EGFR is a substrate of RPTPP/, lysates from PTN-stimulated HUVS cells that express EGFR were incubated with GST-RPTPβ/ζ D1, the “active” active site domain of RPTPβ/ζ, GST-RPTPβ/ζ D1 (C1932S), the “inactive” active site domain of RPTPβ/ζ, and GST-RPTPβ/ζ D1 (D1900A), the “substrate trap” mutant, that captures the substrate phosphoryl-intermediate in the active site of RPTPβ/ζ with high affinity and specificity (Dewang, Hsu et al. 2005, supra). Each of the D1 domain fragments of RPTPβ/ζ “captured” EGFR (FIG. 10B), supporting the thought that EGFR is a substrate of RPTPβ/ζ. However, since GST-RPTPβ/ζ D1 (D1900A) “captured” EGFR, the data strongly support EGFR is a substrate of RPTPβ/ζ. The data suggest EGFR is regulated through the alternative mechanism of RTK activation that regulates activation of Anaplastic Lymphoma Kinase (ALK) as we previously described (Perez-Pinera et al. 2007, supra).

### PTN Activates Akt, Inactivates GSK3β, and Decreases Steady State Levels of Serine 33, 37 and Threonine 41 Phosphorylation in β-Catenin in PTN Stimulated Cells:

EGF stimulates β-catenin transcriptional activity through EGFR (Fang, Hawke et al. 2007, J Biol Chem 282(15): 11221-9). As mentioned above, EGFR activated by EGF activates PI3K, which, in turn, mediates phosphorylation of Akt threonine 308 and serine 473, leading to activation of Akt. Activated Akt, in turn, phosphorylates glycogen synthase kinase 3β (GSK3β) at serine 9 and inactivates its catalytic activity. The pathway of β-catenin import into nuclei through inactivation of GSK3β prevents phosphorylation of serines 33 and 37 and threonine 41 in β-catenin, and β-catenin is not ubiquitinated; it accumulates in cytosol and is imported into nuclei (Henderson and Fagotto 2002, EMBO Rep 3(9): 834-9). When not inactivated by phosphorylation of serine 9, GSK3β prevents phosphorylation of serines 33 and 37 and threonine 41 which target β-catenin for ubiquitination and its degradation through the ubiquitin proteosome proteolysis pathway.

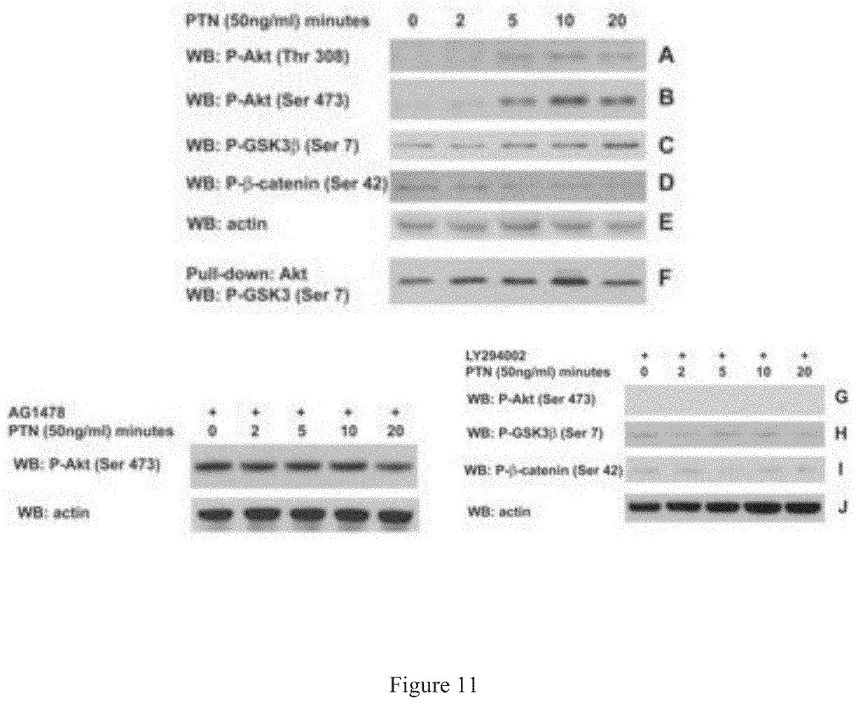

To pursue this pathway of nuclear import of β-catenin, HUVS cells were stimulated with PTN (50 ng/ml), lysed, and analyzed in Western blots probed with anti-phosphothreonine 308 Akt or anti-phosphoserine 473 Akt antibodies (FIG. 11, panel A and B). Phosphorylation of threonine 308 and serine 473 in Akt activate Akt. A rapid increase in phosphorylation of Akt threonine 308 and serine 473 was found 5 minutes after HUVS cells were stimulated with PTN and remained elevated at 20 minutes, indicating Akt is activated in PTN-stimulated cells.

Lysates from HUVS cells stimulated with PTN as above were then analyzed in Western blots probed with anti-phosphoserine 9 GSK3β antibodies. GSK3β is known to be phosphorylated by Akt at serine 9 and to inactivate its catalytic activity. A striking increase in GSK3β serine 9 phosphorylation was observed 5, 10, and 20 minutes after the cells were stimulated with PTN (FIG. 11, panel C), suggesting phosphorylation of Akt threonine 308 and serine 473 has activated Akt, which, in turn, has phosphorylated GSK3β serine 9. Lysates were then prepared from HUVS cells stimulated with PTN for 2, 5, 10 and 20 minutes and immunoprecipitated with anti-Akt antibodies. The immunoprecipitates were incubated with a recombinant peptide of GSK3β amino acid residues 1-22 and ATP. Phosphorylation of the recombinant GSK3β peptide 1-22 was readily detected in an in vitro kinase assay using Akt immunoprecipitated from lysates of cells stimulated with PTN for 2 minutes; peak phosphorylation levels were seen with immunoprecipitates from cells stimulated with PTN for 10 minutes (FIG. 10, panel F). The data provide direct support Akt activated in PTN-stimulated cells phosphorylate GSK3β serine 9.

A key function of activated GSK3β in the pathway of β-catenin translocation into the nuclei is to phosphorylate β-catenin serines 33, 37 and threonine 41. Phosphorylation of serine 33, 37, and threonine 41 in β-catenin targets β-catenin for ubiquitination and degradation through the ubiquitin proteasome proteolytic pathway. In contrast, when GSK3β is inactivated, β-catenin no longer is targeted for ubiquitination and proteolysis. The levels of phosphorylation of serine 33, 37 and threonine 41 in β-catenin were therefore measured in Western blots of lysates from PTN-stimulated cells probed with anti-β-catenin antibodies that recognize phosphoserines 33, 37 and threonine 41. We found levels of phosphorylation of serines 33, 37 and phospho-threonine 41 in β-catenin were sharply decreased 2 minutes after cells were stimulated with PTN; furthermore, phosphorylated serines 33, 37 and threonine 41 in β-catenin were effectively imperceptible during the remaining course of the experiment. In the control study, β-catenin phosphorylated in serines 33, 37 and threonine 41 was readily detected when the cells are not stimulated with PTN. The data thus are consistent that GSK3β is inactivated in PTN-stimulated cells and thus β-catenin no longer is phosphorylated at serines 33, 37 and threonine 41.

To then determine the role of activation of EGFR in the PTN-stimulated activation of Akt, HUVS cells were preincubated with AG1478, a potent and specific EGFR inhibitor, and HUVS cells treated with AG1478 were then stimulated with PTN for 2, 5, 10, and 20 minutes to measure levels of phosphorylation of Akt in Western blots probed with anti-phospho-Akt serine 473 (FIG. 11 B). Inhibition of the EGFR kinase effectively prevented the PTN-stimulated activation of Akt, linking the PTN activation of and activation of EGFR.

The association of adaptor proteins that associate with specific phosphorylated sites in activated EGFR are responsible for the activation of phosphatidyl-inositol 3-kinase (PI3K). HUVS cells were then incubated with the PI3K inhibitor LY294002 for one hour. The cells were stimulated with PTN for 2, 5, 10, and 20 minutes, cell lysates were prepared, and the lysates were analyzed in Western blots probed with anti-phospho-Akt (Ser473), anti-phospho-GSKβ (Ser9), and anti-phospho-β-catenin (Thr42) antibodies. The results demonstrate inhibition of PI3K by LY294002 prevents the PTN-stimulated phosphorylation of Ser473 in Akt, Ser9 in GSK3β, and threonine 41 in β-catenin. This experiment, coupled with the experiments presented above, indicate a unique new PTN-stimulated pathway. Together, the data demonstrate EGFR is activated through the PTN/RPTPβ/ζ “alternative” pathway of RTK activation, which, in turn, leads to activation of Akt, inactivation of GSK3β, and stabilization of β-catenin through failure of phosphorylation of serines 33 and 37 and threonine 41 (FIG. 11C).

SW-13-Ptn Cell Nuclei have Increased Levels of β-Catenin and γ-Catenin:

As described above, the PTN-stimulated pathway to block ubiquitination of β-catenin is rapid; it is highly effective 15 and 30 minutes after cells are stimulated with PTN, but levels of ubiquitinated β-catenin had returned to levels found in unstimulated cells when cells were stimulated with PTN for 60 minutes. We thought the transcriptional profile upregulated in PTN-stimulated cells likely also was transient and likely consistent with the EMT induced during development by PTN. However, ectopic Ptn in SW-13-Ptn cells induces phenotypic progression of the SW-13 cells to the aggressive phenotype characteristic of the EMT, a phenotype characteristic of highly aggressive malignant cells (Zhang, Zhong et al. 1999, supra). Thus constitutive PTN signaling in SW-13-Ptn cells led to an “arrested EMT”, and the transcriptional profile of the SW-13-Ptn cells is a reasonable approximation of the transcriptional profile of the PTN-stimulated cells. For this reason, the following studies were done with SW-13-Ptn cells and not with HUVS cells stimulated with PTN.

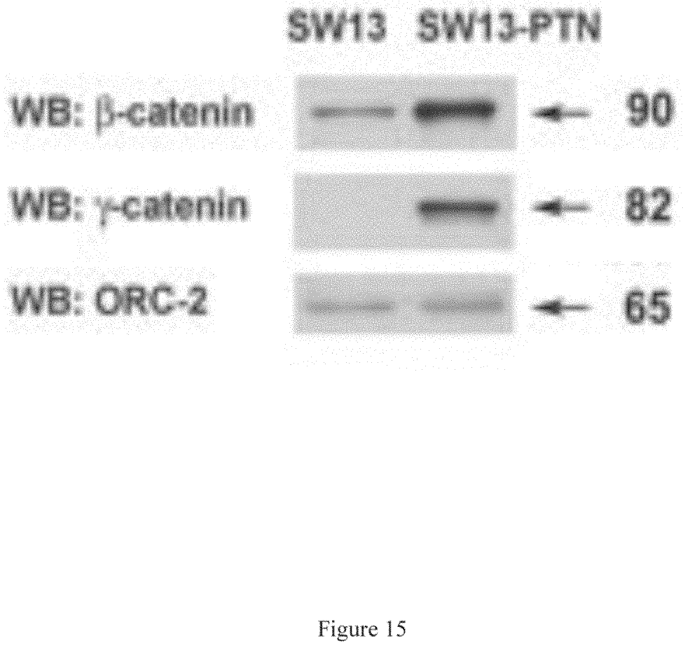

To determine if constitutive PTN signaling in SW-13-Ptn cells also stimulates import of β-catenin into nuclei of SW-13-Ptn cells, lysates of nuclei isolated from SW-13 and SW-13-Ptn cells were analyzed in Western-blots probed with anti-β-catenin antibodies. The levels of β-catenin in nuclei of SW-13-Ptn cells were markedly increased (FIG. 15). In a control study, the levels of ORC-2, a protein found only in nuclei and not known to be influenced by PTN, were compared. The levels of ORC-2 were essentially identical in lysates of SW-13 and SW-13-Ptn cell nuclei.

The levels of γ-catenin also were compared in nuclear extracts from SW-13 cells and SW-13-Ptn cells; Western-blots of SW-13-Ptn cells nuclear extracts probed with anti-γ-catenin antibodies readily identified γ-catenin in nuclei of SW-13-Ptn cells. Gamma-catenin was not detected in nuclei of SW-13 cells that do not express Ptn.

Our data indicate that nuclei from the more malignant SW-13-Ptn cell with inappropriate expression of Ptn accumulate significantly more β-catenin and γ-catenin. The data furthermore indicate the SW-13-Ptn cell was a reasonable approximation of the EMT known to be induced by PTN in PTN-stimulated cells. In this context, we believe the motile, invasive phenotype of malignant cells with aberrant Ptn have a significant overlap with the EMT induced by PTN in PTN-stimulated cells.

β-Catenin in Nuclei of SW-13-Ptn Cells Binds Differentially to Members of the Tcf/Lef Family than SW-13 Cells:

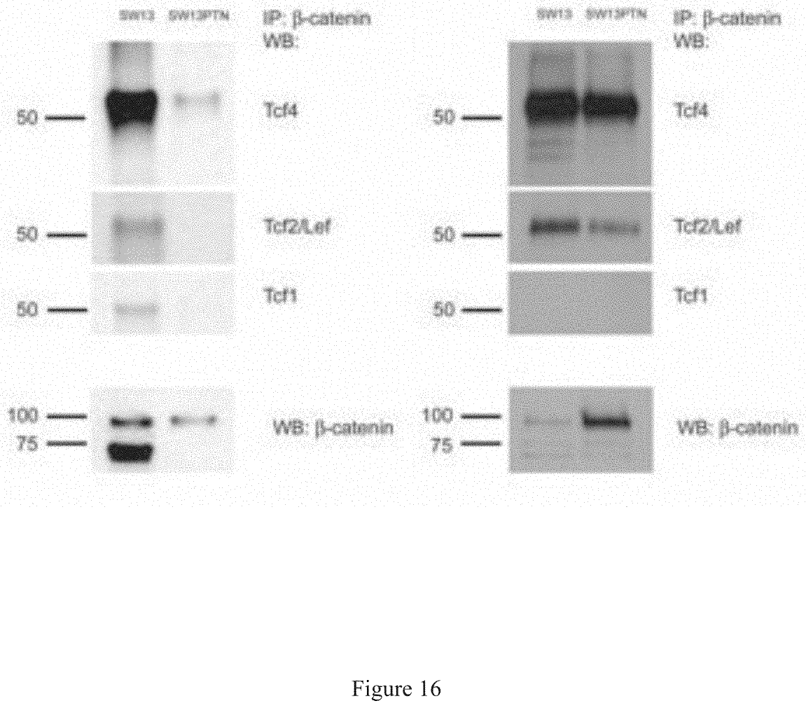

β-catenin in nuclei is known to interact with transcription factors of the Tcf/Lef family and through these interactions stimulate transcriptional activation of genes implicated in development, differentiation, and malignant transformation. To test whether β-catenin in nuclei of SW-13-Ptn cells interacts with Tcf/Lef proteins, β-catenin was immunoprecipitated from nuclear lysates of SW-13 and SW-13-Ptn cells with anti-β-catenin antibodies. The immunoprecipitates were analyzed in Western-blots probed with anti-Tcf1, anti-Tcf2/Lef, anti-Tcf4 antibodies, and anti-β-catenin antibodies (loading control) (FIG. 16). Surprisingly, β-catenin in nuclei of SW-13 and SW-13-Ptn cells migrated at estimated molecular weights of 92 kDa, 75 kDa, and 67 kDa. The three isoforms were immunoprecipitated in roughly equally levels in SW-13 cells; however, in SW-13-Ptn cells, the full-length 92 kDa β-catenin was predominant over the other isoforms and strikingly higher in concentration than in SW-13 cells. Levels of the 75 kDa and 67 kDa isoforms immunoprecipitated in equal levels in SW-13 and SW-13-Ptn cells. Densitometric analysis of the bands obtained in Western-blots of β-catenin immunoprecipitates probed with anti-Tcf4 antibodies and corrected according to the β-catenin loading control demonstrated the level of Tcf4 associated with β-catenin is about 5.5-fold higher in nuclei of SW-13 cells than in nuclei of SW-13-Ptn cells. Similar analysis of Tcf2/Lef binding to β-catenin demonstrated Tcf2/Lef associated with β-catenin is about 11.5-fold higher in SW-13 than in SW-13-Ptn cells. When amounts of lysates were adjusted to apply equal quantities of full-length β-catenin and Western blots were probed with anti-Tcf1, anti-Tcf2/Lef or anti-Tcf4 antibodies, we confirmed that equal amount of full-length β-catenin co-immunoprecipitates with more Tcf1, more Tcf2/Lef and more Tcf4 in SW-13 cells than co-immunoprecipitates in SW-13-Ptn cells. Although others have described alternative splice forms of β-catenin in malignant cells, the basis of the differential association of β-catenin with Tcf/Lef family was not explained and not pursued further.

Tcf/Lef Induced Transcriptional Activity is Higher in SW-13-Ptn Cells than in SW-13 Cells:

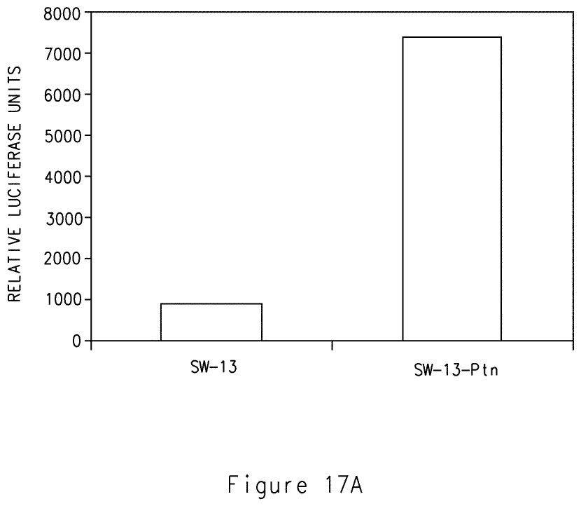

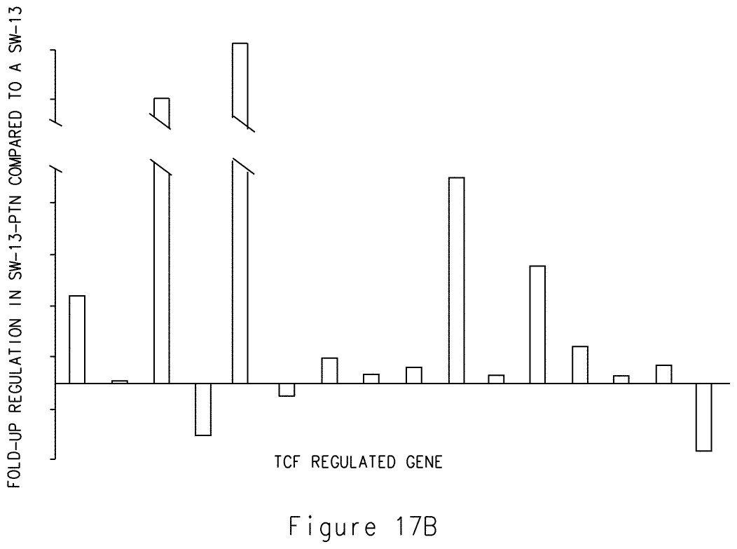

β-catenin import into nuclei leads to up-regulation of the transcriptional activity of the Tcf2/Lef family (Mulholland, Dedhar et al. 2005, Endocr Rev 26(7): 898-915). The association of β-catenin with the Tcf/Lef family members “switches” the Tcf/Lef family from transcriptional repression to transcriptional activation (Mulholland, Dedhar et al. 2005, supra). To test the possibility transcription initiated by members of the Tcf/Lef family is increased in SW-13-Ptn cells, the vector “Top-Flash”, which contains the fire-fly luciferase gene downstream of the Tcf/Lef DNA recognition sequences, was introduced into SW-13 and in SW-13-Ptn cells together with the pSV-β-gal vector to encode β-galactosidase (FIG. 17). The luciferase activity in SW-13-Ptn cells is about 5-fold higher than the luciferase activity in SW-13 cells; stable expression of an activated Ptn gene not only imports β-catenin into nuclei of SW-13-Ptn cells but it induces higher levels of β-catenin mediated transcriptional activity mediated through the Tcf/Lef DNA recognition sequences.

Tcf/Lef Regulated Gene mRNA Levels are Up-Regulated in SW-13-Ptn Cells:

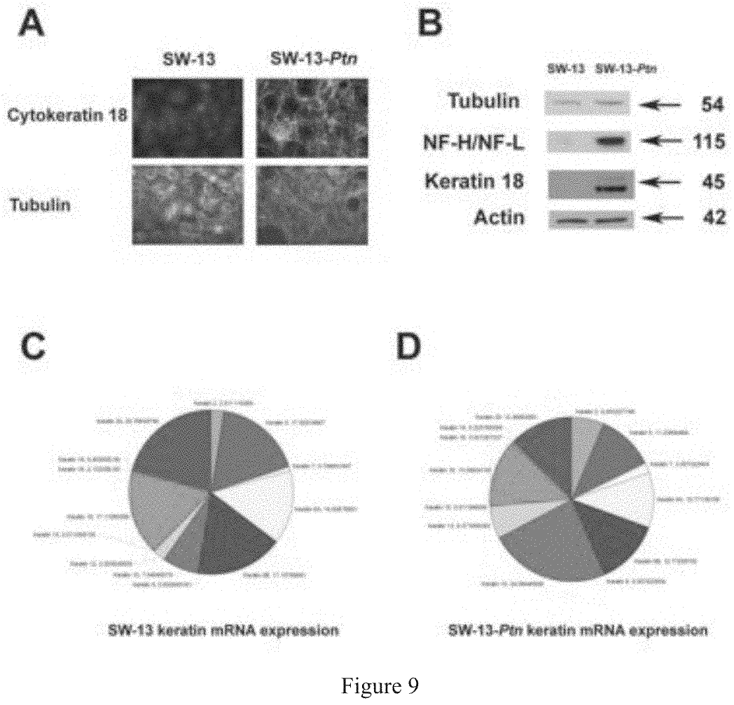

The transcriptional activity induced by the association of β-catenin with the Tcf/Lef family members regulates the activity of different genes, many of which have been implicated in epithelial to mesenchymal transitions during development and in malignant transformation. To investigate whether accumulation of β-catenin in nuclei of SW-13-Ptn cells and its association with Tcf/Lef proteins cited above contributes to transcription of known Tcf/Lef regulated genes and, potentially also to the highly malignant phenotype previously described in SW-13-Ptn cells (Zhang, Zhong et al. 1999, supra), quantitative real time RT-PCR was used to measure the mRNA levels of Tcf/Lef regulated genes. The results were compared with the respective mRNA levels in SW-13 cells. We found an up-regulation of the mRNA encoding MDR1 (about 40-fold), ZO1 (about 1.8-fold), uPAR (about 83-fold), c-jun (about 17-fold), survivin (about 1.8-fold), DRCTNNB1A (about 1.7-fold), peroxisome proliferator activated-receptor δ (about 1.1-fold), Id2 (about 5-fold), TCF-1 (about 7-fold), Brachyury (about 3.7-fold), NBL4 (about 23-fold), c-myc (about 3.4-fold), and ITF-2 (about 65-fold) in SW-13-Ptn cells compared with SW-13 cells. Cyclin D1 (about 13-fold), Fra-1 (about 2-fold) and connexin-43 (about 10-fold) mRNA levels were down-regulated in SW-13-Ptn cells compared with SW-13 cells (FIG. 9). MMP-7 and gastrin mRNAs also were tested; neither gene was detected either in SW-13 or in SW-13-Ptn cells. Since a number of these genes have major roles in differentiation during embryonic and early neonatal development and in the progression of different human malignancies, we think differential expression of these is likely to contribute to the more aggressive phenotype observed in SW-13-Ptn cells both in vitro and in vivo.

### Discussion

The Effect of the PTN Block in Ubiquitination of β-Catenin is Transient, Consistent with a Limited Time Frame for Influence of β-Catenin Functions within Nuclei of PTN-Stimulated Cells:

Pleiotrophin signals through inactivation of the endogenous protein tyrosine phosphatase activity of RPTPβ/ζ, a unique mechanism of signal transduction. As a consequence, PTN increases tyrosine phosphorylation of the different substrates of RPTPβ/ζ; it does so since tyrosine kinases continue to phosphorylate at the same sites that are dephosphorylated when RPTPβ/ζ is still active in PTN-stimulated cells. Pleiotrophin was the first natural ligand to be discovered for this class of receptor type transmembrane tyrosine phosphatases and, thus, the PTN/RPTPβ/ζ signaling pathway triggered by PTN is unique. Thus, the PTN/RPTPβ/ζ signaling pathway regulates tyrosine phosphorylation of key proteins in different complex cellular systems. The first target of the of RPTPβ/ζ discovered was β-catenin (Meng, Rodriguez-Pena et al. 2000, supra); subsequently, β-adducin, histone deacetylase (HDAC)-2 (see below), p190Rho-GAP, GIT1-Cat, and Fyn were identified; the steady state levels of tyrosine phosphorylation of these downstream targets are regulated by the activity of RPTPβ/ζ and sharply increased when RPTPβ/ζ is inactivated in PTN stimulated cells).

Recently, we showed that ALK (Perez-Pinera, Zhang et al. 2007, supra) is activated through the PTN/RPTPβ/ζ signaling pathway; ALK was activated without an interaction of ALK with a known ligand. We found that RPTPβ/ζ maintains the steady state levels of tyrosine phosphorylation of the tyrosine in the activation loop of ALK and thus regulates the catalytic activity of ALK. The EGF receptor family is comprised of four homologous receptors: the epidermal growth factor receptor (ErbB1/EGFR/HER1), ErbB2 (HER2/neu), ErbB3 (HER3), and ErbB4 (HER4). The ErbB receptors become activated by dimerization between two identical receptors (homodimerization) or between different receptors of the same family (heterodimerization) (Lemmon and Schlessinger, 1994, Trends Biochem Sci 19(11): 459-63). The mechanisms that promote the formation of receptor dimers include ligand binding and high receptor density due to overexpression (Lemmon and Schlessinger 1994, supra). In this Example, EGFR is shown to be activated through the PTN/RPTPβ/ζ signaling pathway. We hypothesize that EGFR is activated through the alternative mechanism of RTK activation using the same mechanism through which ALK is activated. This potentially is an important finding that we think applies to other members of the EGFR family, i.e., they are likely also are activated through this mechanism. This finding also may be important since activation of EGFR leads to recruitment and phosphorylation of several intracellular substrates, stimulating to mitogenic signaling and other cellular activities. A major signaling pathway of the ErbB family is the Ras-Raf-MAP-kinase pathway. Another important pathway in erbB receptor signaling is the one constituted by PI-3-kinase and the downstream protein kinase Akt (Muthuswamy, Gilman et al. 1999, Mol Cell Biol 19(10): 6845-57). After its activation, Akt transduces signals that regulate multiple biological processes including apoptosis, gene expression, and cellular proliferation. EGFR signaling is also regulated by phosphatases that maintain steady state levels of tyrosine phosphorylation and activation of EGFR such as PTP-1 B, RPTPκ, or Cdc25A.

In this Example, we describe an entirely new basis for the diversity of cellular responses to PTN signaling; PTN was found to activate Akt and, in turn, Akt was subsequently found to inactivate GSK3β. These findings are important, since, GSK3β is very important in the turnover of β-catenin. The cytosolic tail of β-catenin associates with the APC/Axin/GSK3β complex. β-catenin is phosphorylated by GSK3β within its N-terminal domain, it is targeted for ubiquitination and degradation through the ubiquitin proteasome proteolytic pathway. However, when GSK3β is inactivated, β-catenin no longer is ubiquitinated and no longer is β-catenin targeted for degradation; thus, inactivation of GSK3β lead to accumulation of β-catenin in the cytosol. β-catenin shares homology with importins and, like the importins, it is translocated to the nucleus when it no longer is targeted for proteolysis. In the nucleus, β-catenin regulates the transcriptional activity of the Tcf/Lef family of transcriptional regulators (Brantjes, Barker et al. 2002, Biol Chem 383(2): 255-61; Hatsell, Rowlands et al. 2003, J Mammary Gland Biol Neoplasia 8(2): 145-58; Perez-Pinera, Zhang et al. 2007, supra).

In this Example, we also show that in PTN-stimulated cells β-catenin no longer is ubiquitinated, and it accumulates in the nucleus when GSK3β was inactivated in PTN-stimulated cells. SW-13-Ptn cells that inappropriately express Ptn were found to have higher levels of β-catenin in nuclei than do SW-13 cells. Furthermore, γ-catenin was not found in nuclei of SW-13 cells but readily detectable levels of γ-catenin were found in nuclei of SW-13-Ptn cells. Gamma-catenin also has been shown to be imported into nuclei where, like β-catenin, it regulates Tcf/Lef transcriptional activity, albeit differently than β-catenin (Kolligs, Kolligs et al. 2000, Genes Dev 14(11): 1319-31) but, due to the scope of the Example, roles of γ-catenin in nucleus of SW-13-Ptn cells was not pursued further.

These findings together support that PTN, through the PTN/RPTPβ/ζ signaling pathway, stimulates the import of β-catenin into the nucleus, activates Tcf/Lef transcriptional activity and modifies the transcription levels of genes initiated by Tcf/Lef. These findings support a sensitive model for PTN to initiate differentiation of different cells in development and the different pre-malignant cells with mutations that activate the Ptn gene.

Tyrosine phosphorylation of β-catenin is known to be regulated by different protein tyrosine kinases and phosphatases (eg, Roura, Miravet et al. 1999, J Biol Chem 274(51): 36734-40). Phosphorylation of tyrosines 86, 194, and 654 in β-catenin is known to be related to the loss of the association of β-catenin with cadherins (Piedra, Martinez et al. 2001, J Biol Chem 276(23): 20436-43). The results in the present Example are thus are consistent with the known ability of increased tyrosine phosphorylation of β-catenin to destabilize cytoskeletal structures and to ablate homophilic cell-cell adhesion through decreased affinity of phosphorylated β-catenin for cadherins.

### Example 3

### Activated ALK in Human Breast Cancer

Activating mutations in Anaplastic Lymphoma Kinase (ALK) are causative in human cancers. We describe in the instant Example activated ALK in human breast cancers and cell lines derived from human breast cancers. Limited sequencing of ALK in breast cancers failed to detect known activating mutations, raising the possibility ALK may be activated through our “alternative mechanism” that is independent of known mutations; that is, ALK can be activated as the consequence of the interaction of Pleiotrophin (PTN) with its receptor, RPTPβ/ζ (Perez-Pinera et al. 2007, supra; new data herein). Since earlier studies demonstrated ectopic expression of a dominant negative PTN reversed the malignant phenotype of MDA-MB-231 human breast cancer cells and prevented MDA-MB-231 cell xenograft growth in nude mice (Zhang, Zhong et al. 1997, supra), and ALK itself is expressed in human breast cancers (Perez-Pinera et al. 2007, supra), the data raised the possibility that ALK, activated through constitutive expression of PTN, may be oncogenic in human breast cancer cells. In pursuit of these questions, we identified a unique, previously unrecognized alternative mechanism of ALK activation (Perez-Pinera et el. 2007, supra) and present evidence in the instant Example that ALK is indeed activated in human breast cancers through this alternative mechanism and functions as an essential oncoprotein for growth of human breast cancer cells. Breast cancer is the second most common type of cancer (World Health Organization International Agency for Research on Cancer (June 2003), World Cancer Report) and the fifth most common cause of cancer death worldwide (World Health Organization, February 2006, Fact sheet No. 297-Cancer), so the instant Example, which supports a new and potentially critically important new role for ALK in human breast cancers, may provide important therapeutic targets to reduce the impact of breast cancer.

### Results:

### ALK is Activated in Human Breast Cancers:

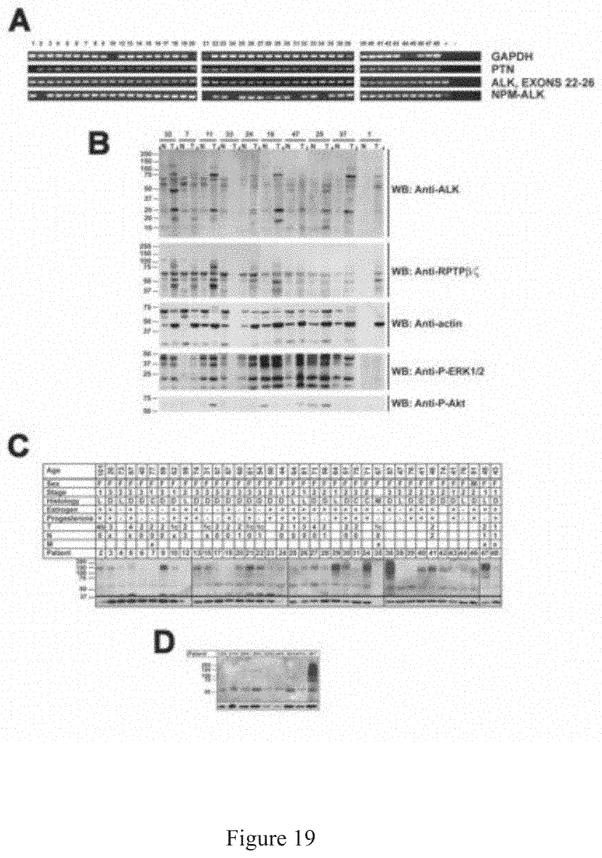

Expression of ALK, PTN, and RPTPβ/ζ were examined using RT-PCR in specimens taken from 46 human breast cancers representative of different pathological stages, both estrogen receptor and progesterone receptor positive and estrogen receptor and progesterone receptor negative. ALK was detected in each of the 46 human breast cancers. Pleiotrophin was detected in 42 of the 46 (FIG. 19A). In previous studies, RPTPβ/ζ had been identified in nearly every human breast cancer analyzed from the same cohort of human breast cancers (Perez-Pinera, Garcia-Suarez et al. 2007, Biochem Biophys Res Commun 362(1): 5-10) and thus was not tested further in this Example.

Lysates prepared from human breast cancers were then analyzed in Western blots probed with anti-ALK antibodies to confirm ALK protein also is expressed in these breast cancer samples. ALK protein was detected in nine of ten samples (FIG. 19B). The Western blots contained a faint band at about 200 kDa, consistent with full-length ALK, and four prominent bands at about 25, about 50, about 80, and about 120 kDa. These patterns of immunoreactive ALK is Western blots were consistent with patterns of immunoreactive ALK reported by others. Activated ALK is known to be degraded through the ubiquitin proteasome proteolytic pathway (Bonvini, Dalla Rosa et al. 2004, Cancer Res 64(9): 3256-64); the results indicate extensive proteolysis may be a feature of ALK in human breast cancers and are consistent with the possibility ALK is activated in these samples of human breast cancer.

To then directly test if ALK is activated in human breast cancers, lysates from 38 human breast cancers were analyzed in Western blots probed with anti-ALK phosphotyrosines 1586/1604 antibodies, antibodies specific for activated ALK. A faint about 200 kDa band consistent with full-length ALK was detected in only few of the 38 samples tested (FIG. 19C); more intensely stained immunoreactive bands were detected at about 140 kDa, about 100 kDa, about 75 kDa, and about 50 kDa. Our data support the human breast cancer cells contain activated ALK; our data also are consistent with extensive proteolysis as anticipated when ALK is activated. Faintly visualized immunoreactive bands at about 200kDa, about 150 kDa, and about 80 kDa were seen in some but not all normal tissues (FIG. 19D), consistent that non-malignant breast tissues also may activate ALK in low levels.

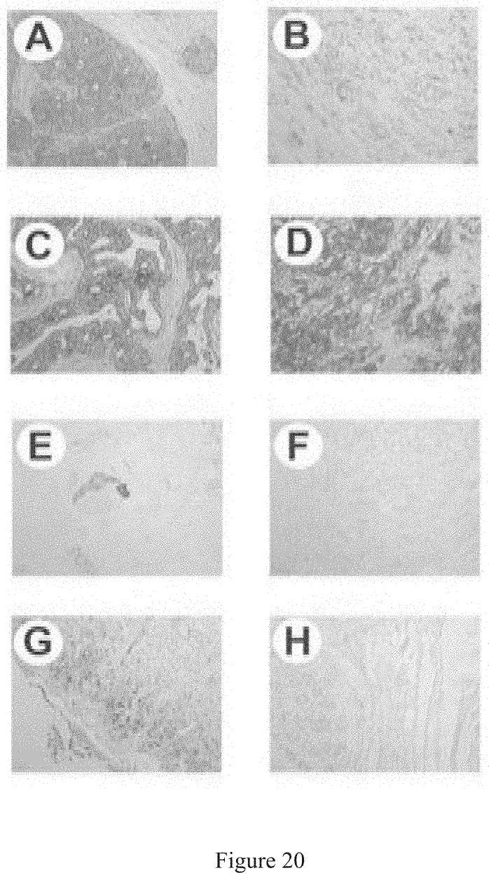

To confirm, we then used a separate sampling of human breast cancers. Anti-ALK phosphotyrosine 1586/1604 antibodies were used to probe a tissue array of 63 samples from 22 subjects with different pathological subclasses of human breast cancers (FIG. 20); activated ALK was recognized in pathologically classified infiltrating lobular carcinoma (B), medullary carcinoma (D), papillary carcinoma (C), mucinous carcinoma (E), and Paget's disease of the breast (G). More limited immunoreactivity was present in ductal carcinomas (A) and, to a much lesser extent, in intraductal carcinomas (F). The distribution of ALK phosphotyrosines 1586/1604 immunoreactivity within individual breast cancer cells differed; a homogeneous distribution was detected in some cells, whereas, in others, a “dot-like” pattern was prominent, and, in yet other cells, it was prominent in nuclei. Importantly, activated ALK also was readily identified in the stromal fibroblasts surrounding the breast cancer cells, albeit at a significantly lesser level, in ductal epithelial cells in normal breast tissue (H).

### Activated ALK is Degraded in Human Breast Cancers:

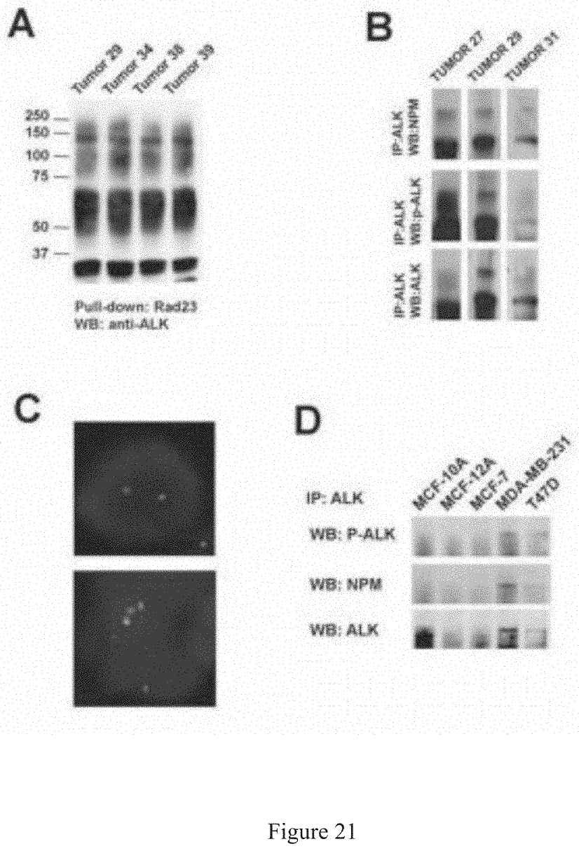

The multiple bands identified with anti-ALK phosphotyrosine 1586/1604 antibodies showed extensive proteolysis of activated ALK, consistent with the known degradation of activated ALK through the ubiquitin proteasome proteolytic pathway (Bonvini, Dalla Rosa et al. 2004, Cancer Res 64(9): 3256-64). Agarose-conjugated Rad-23 was incubated with lysates of human breast cancers to capture mono- and poly-ubiquitinated proteins, which then were analyzed in Western blots probed with anti-ALK antibodies. Rad23 captured multiple proteins that migrated at different mobilities that were recognized by anti-ALK antibodies in Western blots from lysates from each breast cancer sample examined. These protein bands corresponded in part with bands identified in Western blots probed with anti-phospho-ALK tyrosines 1586/1604 antibodies described above (FIG. 21A). The data provide additional support ALK is not only activated but extensively degraded through the ubiquitin proteasome proteolysis pathway in human breast cancers.

### ALK is Activated Through the PTN/RPTPβ/ζ Signaling Pathway in MDA-MB-231 Cells:

To test whether ALK is activated through the PTN/RPTPβ/ζ signaling pathway in MDA-MB-231 cells, lysates from MDA-MB-231 cells, stably transfected with dominant negative PTN, were first analyzed in Western blots probed with anti-ALK and with anti-ALK phosphotyrosine 1586/1604 antibodies specific for activated ALK (Bai, Dieter et al. 1998, Mol Cell Biol 18(12): 6951-61). Anti-ALK phosphotyrosines 1586/1604 immunoreactive proteins were only identified in lysates from MDA-MB-231 empty vector cells, consistent with results above and supporting ALK is activated in cells with an activated PTN/RPTPβ/ζ signaling pathway. Anti-ALK phosphotyrosines 1586/1604 immunoreactive proteins were essentially non-identifiable in Western blots of lysates from aberrant PTN signaling and an activated PTN/RPTPβ/ζ signaling pathway with activation of ALK expressing dominant negative PTN, indicating ALK is not activated when the PTN/RPTPβ/ζ signaling pathway is blocked by dominant negative PTN. However, ALK and multiple lower molecular weight proteolytic immunoreactive forms were seen in lysates from both MDA-MB-231 cells stably transfected with the dominant negative PTN and MDA-MB-231 cells with the empty vector control cells in Western blots probed with anti-ALK antibodies.

Lysates from these human breast cancers also were tested in Western blots with antibodies to detect RPTPβ/ζ, and to detect phospho-ERK1/2, and phospho-Akt known downstream targets of activated ALK (Stoica, Kuo et al. 2001, J Biol Chem 276(20): 16772-9). Consistent with previous studies, multiple bands, likely to be proteolytic fragments of RPTPβ/ζ, were detected. Multiple bands identified as phospho-ERK1/2 and P-Akt also were detected, consistent but not specific that ALK is activated in human breast cancers.

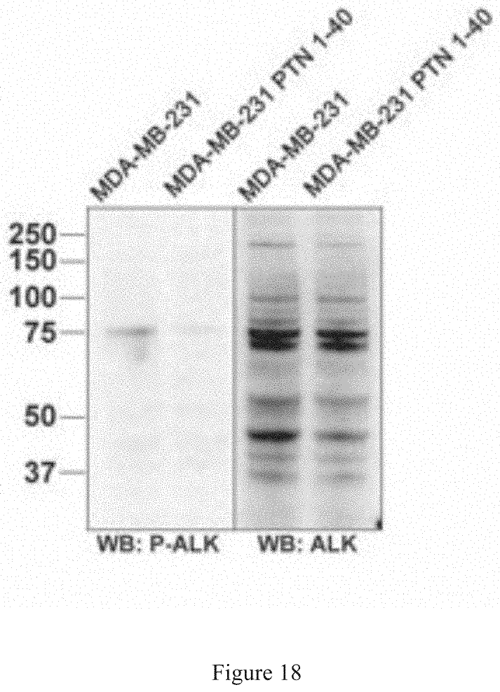

Our data thus indicate ALK is activated in MDA-MB-231 cells with an intact PTN/RPTPβ/ζ signaling pathway (FIG. 18) but not when the PTN/RPTPβ/ζ signaling pathway is disrupted by the dominant negative PTN. The data are consistent that ALK is activated in human breast cancer cells through the alternative mechanism dependent on the PTN/RPTPβ/ζ signaling pathway and independent of mutations in ALK itself. Furthermore, since dominant negative PTN not only blocks activation of ALK but reverts the transformed of MDA-MB-231 cells to the non-transformed phenotype, the data support the probability that ALK activated through the PTN/RPTPβ/ζ signaling pathway is an important driver of the transformed phenotype of the human MDA-MB-231 cells.

The c-met/ALK Dual Inhibitor PF2341066 Blocks Growth of MDA-MB-231 and T47D Breast Cancer Cells:

The data above support that ALK, activated through the PTN/RPTPβ/ζ signaling pathway, may be an important driver of the transformed phenotype of MDA-MB-231 cells. To determine whether activated ALK is essential for proliferation of human breast cancer cells, cDNAs prepared from MCF10A, MCF12A, MCF7, MDA-MB-231, and T47D human breast cancer cells were analyzed with RT-PCR using primers to specifically identify ALK. The transcripts of ALK were identified in each of the 5 cell lines. Lysates from each cell line were then immunoprecipitated with anti-ALK antibodies and analyzed in Western blots probed with anti-phosphotyrosine antibodies or with anti-ALK antibodies.

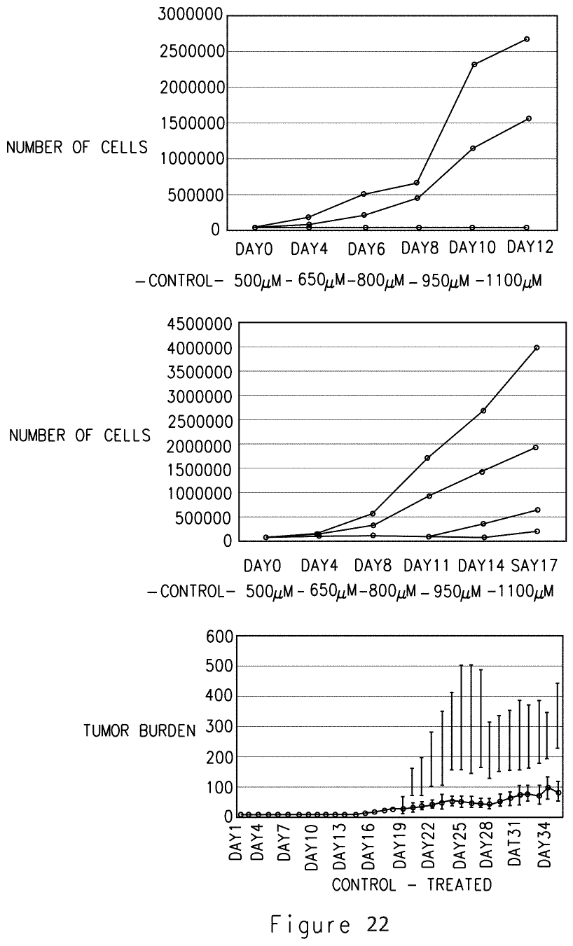

The MDA-MB-231 (FIG. 22A) and T47D (FIG. 22B) cells were then treated with the ALK inhibitor PF2341066. PF2341066 induced a dose-dependent block in proliferation of both MDA-MB-231 cells and T47D cells in culture (FIG. 22A). At 1 μM, PF2341066 effectively prevented growth of MDA-MB-231 cells and reduced T47D cell growth about 13-fold.

### Inhibition of Activated ALK Reduces MDA-MB-231 Xenograft Growth in Flanks of Nude Mice:

To then test whether ALK is a driver of human breast cancer cell growth in vivo, MDA-MB-231 cells were injected into flanks of nude mice and MDA-MB-231 cell xenograft growth observed in nude mice treated with the ALK inhibitor PF2341066 or, in control mice, treated with the vehicle alone. Both groups developed palpable tumors; however, the mice treated with PF2341066 had an about 3.5-fold reduction of tumor burden at day 22 and an about 3-fold reduction at the end of the experiment (FIG. 22C). The data support activated ALK is an important driver of human breast cancer cell xenograft growth in vivo as well as that inhibition of activated ALK, or regulators of ALK like PTN and RPTPβ/ζ, are potential therapy targets for human breast cancer.

### Discussion

The instant Example directly demonstrates that the tyrosine kinase, ALK, is activated in a large number of human breast cancers. They also demonstrate the chemical inhibitor PF2341066 effectively blocks growth of human breast cancer cells in which ALK is activated, both in culture and in human breast cancer cell xenografts in nude mice. These studies furthermore demonstrate both activation of ALK and the malignant phenotype of breast cancer cells require activation of the PTN/RPTPβ/ζ signaling pathway; loss of activation of ALK and loss of the malignant phenotype of breast cancer cells result when the PTN/RPTPβ/ζ signaling pathway is blocked through dominant negative PTN. The data thus demonstrate that ALK is an essential driver of the breast cancer phenotype in breast cancer cells in which it is activated; the data also support the conclusion ALK is activated in the breast cancer cells through our alternative mechanism of ALK activation mediated by the PTN/RPTPβ/ζ signaling pathway (Perez-Pinera et al. 2007, supra). These studies thus support the potentially highly significant new breakthrough that ALK may be activated in human breast cancers through alternative mechanisms that do not depend on activating mutations in ALK itself. ALK is an essential driver of growth and the malignant phenotype of some breast cancer cells and activated ALK is likely an important biomarker in human breast cancers.

A potentially important additional observation was activated ALK was identified in the stromal fibroblasts that surround the breast cancer cells in the different breast cancers examined. In earlier studies, PTN secreted from breast cancer cells was found sufficient to activate stromal fibroblasts, perhaps in part through activation of ALK (Chang, Zuka et al. 2007, Proc Natl Acad Sci USA, 104(26): 10888-93). Activated ALK transforms fibroblasts (Soda, Choi et al. 2007, Nature 448(7153): 561-6), the tumor promoting functions of the activated fibroblast may be the result of pathways downstream of ALK in those breast cancers.

The different downstream pathways activated by ALK provide a strong basis to conclude activated ALK when detected in breast cancers may have high impact in the progression of human breast cancers. ALK is potently oncogenic; activated pathways downstream of ALK stimulate deregulated cell growth, escape from cell death, and other hallmarks of the cancer cell essential for invasion and metastasis. Pathways stimulated by ALK include phospholipase C-y (Bai, Dieter et al. 1998, Mol Cell Biol 18(12): 6951-61), phosphatidylinositol 3-kinase (Stoica, Kuo et al. 2002, J Biol Chem 277(39): 35990-8), the ERK pathway (Powers, Aigner et al. 2002, J Biol Chem 277(16): 14153-8), insulin receptor substrate 1 (Kuo, Stoica et al. 2007, Oncogene 26(6): 859-69), and diacyl glycerol kinase (Bacchiocchi, Baldanzi et al. 2005, Blood 106(6): 2175-82). ALK is known to form stable complexes with hyperphosphorylated ShcC and to function as a survival factor when activated in the malignant cell. As described in Example 1 above, we have shown that β-catenin is a substrate of ALK, and, phosphorylation of β-catenin by ALK was shown to induce loss of homophilic cell-cell adhesion and an epithelial to mesenchymal transition (EMT), critical steps in the progression of malignant cells to a highly malignant phenotype (Thiery 2000, 2002, supra). Added to these different mechanisms, our discovery (described herein) of a new oncogenic pathway that is dependent on phosphorylation of β-catenin by activated ALK, preventing the association of β-catenin and cadherin, blocking cell-cell adhesion and initiating an epithelial-mesenchymal transition (EMT) with loss of E-cadherin function. Since the EMT in malignant cells induces a highly malignant phenotype, the Examples herein provide a very strong basis for an additional powerful oncogenic stimulus of ALK in the causation of malignant cancers.

Our other recent studies demonstrated that ALK is activated through an “alternative mechanism of receptor protein tyrosine kinase activation” mediated by the PTN/RPTPβ/ζ signaling pathway (Perez-Pinera, Zhang et al. 2007, supra). The discovery potentially is of major significance, since, to that date, ALK was an “orphan receptor” in mammals. That is, the requirement of an interaction of an extracellular ligand of ALK to activate ALK itself was by-passed. Further, we showed that ALK can be activated through the alternative mechanism in malignant cells that have acquired aberrant (constitutive) PTN signaling and activation of the PTN/RPTPβ/ζ signaling pathway. As mentioned above, aberrant PTN signaling has been identified in many malignant cells (Perez-Pinera, Chang et al. 2007, supra), including human breast cancer cells (Zhang, Zhong et al. 1997, supra; Lu, Jong et al. 2005, J Biol Chem 280(29): 26953-64; Perez-Pinera, Chang et al. 2007, supra; Polytarchou, Hatziapostolou et al. 2008, J Biol Chem 277(16): 14153-8). Dirks et al (Dirks, Fahnrich et al. 2002, Int J Cancer 100(1): 49-56) demonstrated expression of ALK in human breast cancer cell lines, and Perez-Pinera et al (Perez-Pinera, Chang et al. 2007, supra) identified ALK protein in many human breast cancers, findings that support ALK is activated and potentially oncogenic in human breast cancers. The PTN/RPTPβ/ζ signaling pathway is constitutively activated in different malignant cancer cells (Zhang, Zhong et al. 1997, supra; Deuel, Zhang et al. 2002, supra; Lu, Jong et al. 2005, supra; Perez-Pinera, Chang et al. 2007, supra; Polytarchou, Hatziapostolou et al. 2008, supra) and, many of these malignant cancer cells also express ALK (Dirks, Fahnrich et al. 2002, supra).

Our studies thus support a very important mechanism through which ALK is activated in human breast cancer cells. Activated ALK may have a significant role in driving proliferation of human breast cancer cells in vitro and in vivo. Our data furthermore support activated ALK, and/or regulators of ALK like PTN and RPTPβ/ζ may be markers in a large number of breast cancers and potentially a therapeutic target in human breast cancers.

### Experimental Procedures:

### Human Breast Cancers:

Different subtypes of human breast cancers were obtained according to the current Spanish Biomedical Research regulations, Law 121/000104. Their use has been reviewed and approved by the Institutional Review Board at The Scripps Research Institute (HSC004690).

### Plasmids:

The vector pcDNA3.1/PTN1-40 was constructed by inserting the cDNA fragment encoding residues −32 to 40 of human PTN protein into XbaI and BamHI of pcDNA3.1/myc-his/hygro vector (Invitrogen).

### Immunohistochemistry:

Breast cancer tissue arrays (Catalog No. CC08-01-005) were obtained from Cybrdi (Frederick, Maryland). Tissue slides were deparaffinized (2×10 min) in xylene, and hydrated (2×10 min) with 100%, 95% (2×10 min), (1×10 min) 90%, (1×10 min) 70% ethanol, and distilled water (10 min). The slides were then incubated in antigen retrieval solution (Trypsin 0.05%, CaCl2 0.1%, pH 7.8) for 20 min at 37° C. and then for 10 min at room temperature in a humidified chamber. Endogenous peroxidase was quenched by incubating the sections with 3% hydrogen peroxide for 5 min and the tissues were permeabilized by incubating the samples in Tris-buffered saline (TBS, 10 mM Tris, pH 7.6, 150 mM NaCl) with 1% Triton X-100 for 30 min. Non-specific binding of the antibodies was reduced by incubating the sections for 30 min in a blocking solution containing 2% bovine calf serum, 2% goat serum, 1% BSA, 0.1% gelatin, 0.1% Triton X-100, 0.05% Tween 20 in 10 mM PBS, pH 7.2. The sections were incubated overnight with anti-ALK antibodies (Zymed, currently Invitrogen, Carlsbad, Calif.) diluted 1:100 in PBS, pH 7.2, 1% BSA, and 0.1% gelatin overnight. The slides were then washed with permeabilization solution (2×10 min), incubated with SuperPicTure polymer from Zymed for 30 min, washed in PBS (2×3 min), and developed with DAB provided with the SuperPicTure kit from Zymed. The slides were rinsed in distilled water 10 min and dehydrated with 70% (1×10 min), 90% (1×10 min), 95% (2×10 min), 100% ethanol (2×10 min), and cleared in xylene (2×10 min), mounted, observed with a Nikon TE2000U microscope coupled with a Confocal Cell Imaging CARV system, and photographed.

### Cell Lines:

MDA-MB-231, MCF-7, and T47D cells were obtained from American Tissue Collection Center (ATCC) and grown in DMEM supplemented with 10% fetal bovine serum (FBS) and 1% penicillin/streptomycin at 37° C. in a 5% CO2 atmosphere. MCF-10A and MCF-12A cells were grown in DMEM supplemented with 10% FBS, hydrocortisone 0.5 mg/ml, 0.5 ml, hEGF 10 ug/ml, 0.5 ml, insulin 5 mg/ml, and 100 ng/ml cholera toxin.

### Antibodies:

Anti-ALK antibodies were obtained from Invitrogen (Carlsbad, Calif.). Anti-phospho-ALK tyrosine 1604, anti-phospho-ALK tyrosine 1586 and anti-NPM were obtained from Cell Signaling Technology (Danvers, Mass.). Anti-actin antibodies were obtained from Sigma-Aldrich (St. Louis, Mo.).

### RT-PCR:

RNA from human breast cancers and human breast cancer cells was extracted using the RNeasy Mini kit obtained from Qiagen, Valencia, Calif., following manufacturer's recommendations. Preparations were treated with DNase obtained from Ambion (Austin, Tex.), reverse transcription performed using the SuperScript First Strand Synthesis System obtained from Invitrogen (Carlsbad, Calif.). The cDNA was treated with Ribonuclease A purchased from Invitrogen and PCR amplification was performed in a 50 μI reaction containing 20 mM Tris-HCl, pH 8.4, 50 mM KCl, 0.2 mM dNTP mixture, 1.5 mM MgCl2, 0.2 μM primer forward, 0.2 IM primer reverse, 2 μI of cDNA from the prior reaction and, 2 units of Platinum Taq DNA polymerase (Invitrogen). Samples were loaded in 1.4% agarose gels containing 0.5 μg/ml ethidium bromide and visualized using a UV transilluminator (BioRad, Hercules, Calif., USA). Appropriate primers were used’. As a negative control reactions with RNA instead of cDNA or without primers were used, whereas as a positive control the expression of GADPH was analyzed.

### Western Blots:

Cell lysates were mixed with loading buffer (60 mM Tris pH 6.8, 10% glycerol, 5% SDS, 0.65% β-mercaptoethanol, and 0.01% bromophenol blue), boiled for 5 minutes, and loaded in 4-16% polyacrylamide gels as appropriate. The gels were transferred to nitrocellulose membranes that were blocked with 50 mM Tris, 150 mM NaCl, 0.1% Tween-20 (TBS-T), and 10% BSA for 1 hour and incubated with the primary antibodies at the dilutions indicated overnight in TBS-T with 10% BSA. After 3 washes in TBS-T, the membranes were incubated for 20 minutes with secondary antibodies conjugated with horseradish peroxidase diluted 1:10,000 in TBS-T with 5% non-fat milk. The membranes were washed 3 times in TBS-T and the immunoreactive proteins visualized using the ECL Enhanced Method (Amersham, San Francisco, Calif.).

### Ubiquitination:

Lysates were prepared in a lysis buffer containing 50 mM Tris, 150 mM NaCl pH 7.5, 1% NP40, 0.25% sodium deoxycholate, 0.1% SDS, Complete EDTA-free Protease Inhibitor Cocktail (Roche, Indianapolis, Ind.), and 2 mM sodium orthovanadate. The lysates were incubated overnight with agarose-beads conjugated with Rad23, a protein that binds specifically to poly-ubiquitinated proteins (Calbiochem, La Jolla. Calif.). The beads were washed 4 times in lysis buffer, boiled in loading buffer containing 240 mM Tris pH 6.8, 40% glycerol, 20% SDS, 2.5% β-mercaptoethanol and 0.01% bromophenol blue and analyzed in Western-Blots.

### Fluorescent In Situ Hybridization:

To assess for rearrangements of the ALK loci, two FISH probes were created to hybridize with the neighboring centromeric and telomeric regions of ALK conjugated with fluorescein and Texas-red obtained from Dako (Carpinteria, Calif.) following the manufacturer's recommendations.

### Xenograft Growth in Flanks of Nude Mice:

Male athymic nude mice (8 weeks old, Cby.Cg-Foxn1nu) were used. Two million MDA-MB-231 cells were injected subcutaneously into the flanks of nude mice. Mice were treated with 50 mg/kg PF2341066 or DMSO as vehicle control. The sites of injection were observed daily for tumor growth beginning 1 day after injection and tumor length (L) and width (W) were measured, and tumor volume was estimate using the formula (LXW2)/2.

Table 2. Expression of ALK in different human breast cancers. The intensity of the immunoreaction was estimated in a scale of 1 to 4 and the average signal for each subtype of breast cancer represented. It was found that 83% of ductal carcinomas, 25% of intraductal carcinomas, and 100% of the lobular carcinomas, medullary carcinomas, papillary carcinomas, mucinous carcinomas, and Paget's disease express ALK that is phosphorylated to some extent.

| | Average Signal | Percent of |
|---|---|---|
| | Intensity | positive tumors |
| Ductal Carcinoma | 2.11 | 83 |
| Lobular Carcinoma | 1.83 | 100 |
| Medullary Carcinoma | 4 | 100 |
| Papillary Carcinoma | 3.5 | 100 |
| Mucinous Carcinoma | 3.66 | 100 |
| Intraductal Carcinoma | 0.6 | 25 |
| Paget's disease | 2.2 | 100 |

### Example 4

### Pleiotrophin Promotes Breast Cancer Progression In MMTV-PyMT Transgenic Mice: Induction of Scirrhous Invasive Ductal Carcinoma-Like Phenotype and Tumor Angiogenesis

Pleiotrophin (PTN, Ptn) is a 15 kDa secreted cytokine frequently expressed in breast cancers and inappropriately expressed in cell lines derived from these breast cancers; nevertheless, the role of PTN in breast cancer is unknown. The instant Example demonstrates that Ptn expression in mouse mammary tumor virus (MMTV) promoter-Ptn single transgenic mice is insufficient to develop breast cancers. However, Ptn expression in MMTV-Polyoma Virus Middle T antigen (PyMT)-Ptn bi-transgenic mice promotes more aggressive breast cancers than in MMTV-PyMT single transgenic mice. The breast cancers contain foci resembling invasive scirrhous ductal carcinomas in humans that express very high level Ptn and exhibit markers characteristic of PTN signaling, indicating inappropriate PTN expression cooperates with other oncogenic proteins to promote a defined phenotype of more aggressive breast cancer.

The present Example is significant, among other reasons, because the molecular mechanisms that underlie the histological subtypes of human breast cancers are poorly understood. This Example demonstrates PTN expression alone does not induce mouse breast cancer but demonstrates that PTN expression cooperates with PyMT expression to promote progression of breast cancer to mimic aggressive invasive scirrhous ductal-like carcinomas in humans. PTN is frequently expressed in human breast cancers and invasive scirrhous ductal carcinomas are among the most aggressive of human breast cancers; the studies in this Example support that initiation of constitutive PTN signaling in breast cancer cells may be an important mechanism and marker of an aggressive histological subtype of breast cancer and support the important possibility that the PTN signaling pathway is a potential target to test for therapy of breast cancer.

Breast cancer continues to be the most common malignancy in women in the United States; it accounts for more than 40,000 deaths each year. The need to better understand the mechanisms that initiate breast cancer and the consequences of different mutations that drive progression of breast cancers to more malignant phenotypes remains of highest priority. Recently, mutations that initiate inappropriate expression of endogenous genes in cancer cells have been recognized as important mechanisms to stimulate tumor progression (Hanahan and Weinberg, 2000, Cell 100, 57-70). Identification of these genes that are inappropriately expressed is important since the association of inappropriately expressed genes with specific subtypes of breast cancer indicate these genes are markers to predict prognosis, and the deregulated pathways that are the consequences of these mutations are potential targets to direct pathway specific therapy.

Pleiotrophin (PTN the protein, Ptn the gene) is a 136 amino acid heparin-binding cytokine whose gene frequently has been detected in different screening studies of human breast cancers (e.g., Perou et al., 2000, Nature 406, 747-752). Inappropriate expression of Ptn is characteristic of cell lines derived from human breast cancers that express Ptn (Wellstein et al., 1992, J Biol Chem 267, 2582-2587; Zhang et al., 1997, supra); constitutive PTN-signaling is important in the pathogenesis of aggressive human breast cancers and, mechanistically, PTN signaling may define their properties. Pleiotrophin expression also is detected in many other human malignant cancers, including neuroblastomas, glioblastomas, and pancreatic and prostate cancers (see Example 10, below); thus, we think it likely that inappropriate expression of Ptn is a significant factor in the progression of many human malignancies.

To better understand the role of inappropriate Ptn expression in pathogenesis of malignant breast cancers, mouse mammary tumor virus (MMTV)-Ptn single transgenic mice were generated. The MMTV promoter has been widely used to specifically direct expression of transgenes into mouse epithelial cells and, through insertional activation, to successfully identify a number of important oncogenes such as Wnt-1 (formerly int-1), Wnt-3, int-2, and hst. The subsequent mating of different transgenic animals that express MMTV directed transgenes has since proven to be a powerful tool to demonstrate genes that induce signaling pathways that cooperate with pathways of known oncogenes that are expressed in breast epithelium through MMTV (Kwan et al., 1992, Mol Cell Biol 12, 147-154).

In the instant Example, we generated transgenic mice into which Ptn and an internal ribosomal entry site (IRES) along with Enhanced Green Fluorescence Protein (EGFP) were driven by the long terminal repeat sequences of MMTV (MMTV-LTR) to test whether Ptn expression in mouse mammary epithelial cells alone is sufficient to initiate breast cancer. MMTV-Polyoma middle T (PyMT) single transgenic mice (Guy et al., 1992, Mol Cell Biol 12, 954-961) were then bred with MMTV-Ptn transgenic mice to test whether signaling pathways induced by MMTV-Ptn and MMTV-PyMT cooperate to promote a more aggressive breast cancer phenotype in MMTV-PyMT-Ptn bi-transgenic mice.

MMTV-PyMT mice were selected since female carriers of the MMTV-PyMT transgene develop palpable mammary tumors by 5 weeks of age and thus the potential impact of Ptn is seen quickly. The adenocarcinomas that develop are multifocal, fibrotic, and involve the entire mammary fat pad. The histological features of these tumors include considerable heterogeneity in histological subtypes and thus breast cancers in MMTV-PyMT mice provide a histological background upon which to identify specific subtypes uniquely enhanced by expression of a second MMTV-driven transgene. Pulmonary metastases are observed in 80-94% of tumor-bearing female mice.

These studies demonstrate that inappropriate expression of MMTV-Ptn in MMTV-PyMT mouse breast cancers initiates a “switch” in MMTV-PyMT-Ptn breast cancers to a more highly aggressive phenotype that closely resembles the scirrhous-patterned invasive ductal carcinoma in human breast cancers.

### Results

### Generation of MMTV-Ptn-IRES-GFP Single Transgenic Mice:

To test whether inappropriate expression of Ptn is sufficient to induce breast cancer in mice, MMTV-Ptn-IRES-GFP transgenic mice were generated by microinjection of the transgenic construct MMTV-Ptn-IRES-GFP (see Materials and Methods) into one-cell embryos (Supplemental FIG. 1, Panel A). Eight founder mice were obtained and bred with wild type FVB/N mice. Five mice transmitted the transgene to the next generation and were used to establish the MMTV-Ptn-IRES-GFP transgenic line. RT-PCR was used to demonstrate a high level of expression of the human Ptn transgene in mammary glands excised from one female and one male mouse from founder line 2 (Supplemental FIG. 1, panel B). Founder line 2 was used to breed the mice used in this study. The transgene was not found in tissues from mice of founder line 1 nor in tissues of FVB/N inbred mice.

### MMTV-Ptn-IRES-GFP Single Transgenic Mice Do Not Develop Breast Cancer:

The female MMTV-PTN-IRES-GFP transgenic mice had normal mammary gland development and normal lactation and developed pups in normal numbers and in size equal to control mouse pups. MMTV-Ptn-IRES-GFP transgenic mice were examined for up to one and half years; they were free of detectable breast cancers. Microscopic examination of breast tissues failed to provide evidence for dysplasic changes.

The data thus demonstrate that inappropriate expression of Ptn under the control of MMTV is not sufficient to initiate breast cancer in mouse breast epithelial cells and thus Ptn is not an oncogene in this context.

### MMTV-PyMT Ptn Bi-Transgenic Mice:

Since inappropriate Ptn expression in mouse breast epithelial cells is not sufficient to initiate breast cancer in mice, MMTV-Ptn-IRES-GFP transgenic mice were bred with MMTV-PyMT transgenic mice. MMTV-PyMT (polyoma virus middle T) mice have been extensively studied (Guy et al., 1992, supra). The onset of breast cancers is rapid in MMTV-PyMT mice; it is frequently detected as early as five weeks after birth and is multifocal. The breast cancers in MMTV-PyMT transgenic mice also grow rapidly as breast cancers. The histological features of the breast cancers in MMTV-PyMT mice are heterogeneous; different stages in the hierarchy of tumor progression from pre-malignant to malignant are often found within a single primary focus of breast cancer. These different stages are comparable to different stages of human breast cancers by histological criteria and thus can be classified as benign or carcinoma in situ and as different subtypes of invasive carcinomas (Lin et al., 2003, Am J Pathol 163, 2113-2126). The MMTV-PyMT breast cancers are highly aggressive but exhibit these different features that morphologically resemble subtypes of human of breast cancers that permit distinction of any properties that result from inappropriate expression of MMTV-Ptn.

Comparison of MMTV-PyMT Single Transgenic Mice with MMTV-PyMT-Ptn Bi-Transgenic Mice:

Significant differences in the time of onset of breast cancers or in the numbers of metastases were not found in breast cancers from MMTV-PyMT single and MMTV-PyMT-Ptn bi-transgenic mice. Significant variability was found in the size of individual breast cancers in both MMTV-PyMT and MMTV-PyMT-Ptn mice. The tumor burden (defined here as the total wet weight of tumors/mouse) in the MMTV-PyMT mice was nearly 20% greater than in MMTV-PyMT-Ptn mice (Table 3) but the average tumor volume/mouse was nearly 2-fold increased in the MMTV-PyMT-Ptn mice (Table 4). Western blot analysis using antibodies that recognize both mouse and human PTN was employed to detect expression of PTN protein in breast cancer extracts from both MMTV-PyMT and MMTV-PyMT-Ptn mice. As anticipated, PTN was expressed in very high levels in breast cancers from MMTV-PyMT-Ptn bi-transgenic mice; surprisingly, however, PTN also was expressed in very high levels in breast cancers of MMTV-PyMT single transgenic mice (FIG. 3, left two lanes), albeit at a level less high than in MMTV-PyMT-Ptn mouse breast cancers (FIG. 3, right 3 lanes), raising the possibility that inappropriate expression of Ptn may be initiated by expression of the MMTV-PyMT transgene.

MMTV-PyMT-Ptn bi-Transgenic Mouse Breast Cancers have Increased Foci of Aggressive Carcinomas, Extensive Extracellular Matrix Remodeling, and Increased New Blood Vessels of Increased Size:

Striking differences in the histological features of the breast cancers in MMTV-PyMT and MMTV-PyMT-Ptn mice were seen. A statistically significant increase in morphologically discrete foci of breast cancer cells that closely resemble scirrhous-patterned invasive ductal carcinoma (Debnath and Brugge, 2005, Nat Rev Cancer 5, 675-688; Tsuchiya and Li, 2005, Med Mol Morphol 38, 216-224) were found in MMTV-PyMT-Ptn bi-transgenic mouse breast cancers compared with MMTV-PyMT mouse breast cancers (Table 5). The areas of the breast cancers that were occupied by the foci of scirrhous-patterned invasive ductal carcinoma-like breast cancer in MMTV-PyMT-Ptn bi-transgenic mouse breast cancers was about 39%, a near doubling of the 22% area occupied by these foci in the MMTV-PyMT mouse breast cancers (P<0.05). The clustered breast cancer cells in these foci were surrounded circumferentially by morphologically identified “carcinoma associated fibroblasts” (CAFs), abundant and densely layered extracellular matrix proteins, and increased new blood vessels of increased size (described subsequently). The scirrhous patterned invasive ductal carcinoma-like foci express very high levels of the MMTV-Ptn transgene:

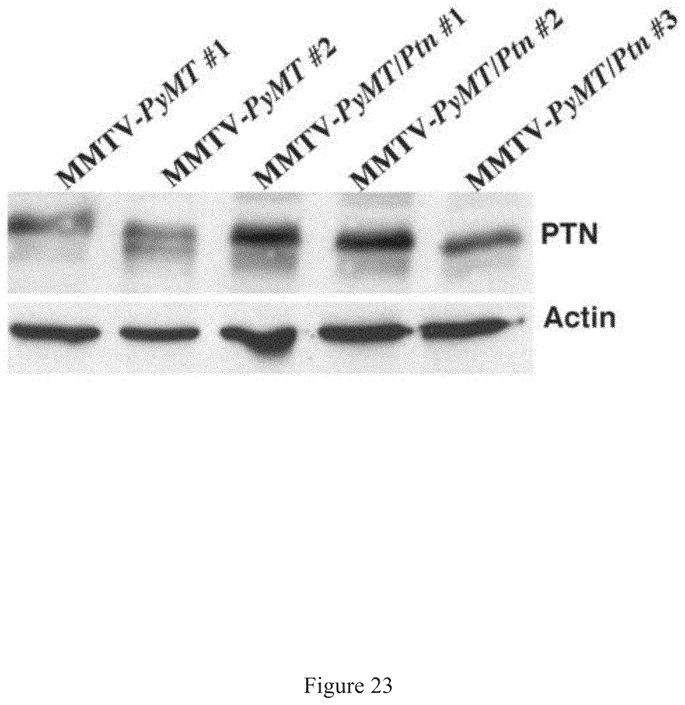

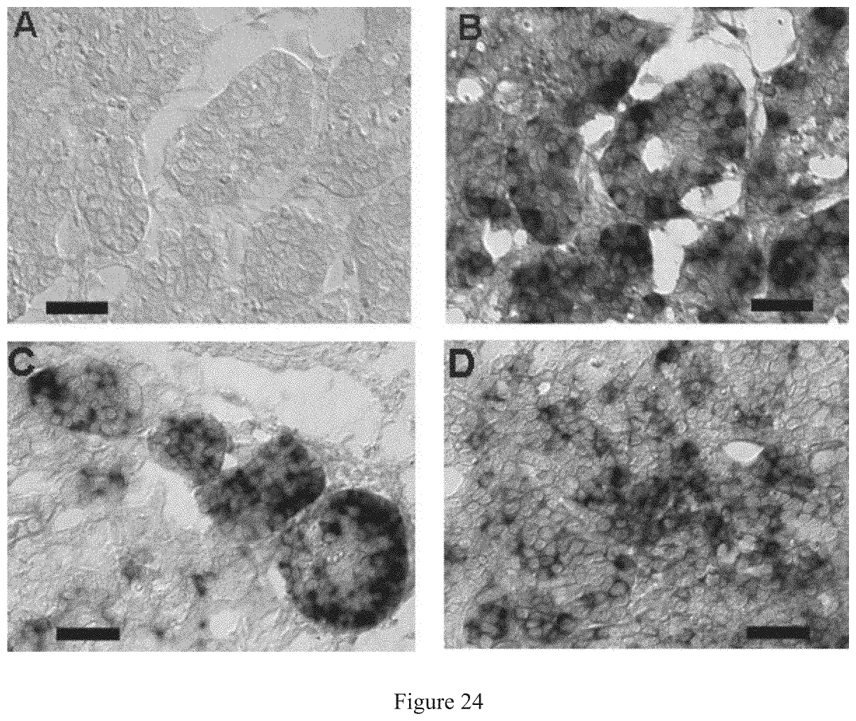

The intensity of the MMTV (human) Ptn transgene expression was examined by in situ hybridization using antisense RNA human Ptn probes was strikingly high within the foci of scirrhous patterned invasive ductal-like carcinomas (B, C and D, FIG. 24). The very high level of the MMTV-Ptn transgene was seen in all of the MMTV-PyMT-Ptn breast cancers examined and was associated exclusively with the foci of the scirrhous-patterned invasive ductal-like carcinomas. The data are consistent with the high level expression of PTN found in Western blots of MMTV-PyMT-Ptn bi-transgenic mouse breast cancers (FIG. 23). However, the data moreover directly correlate the very high level of Ptn expression with the distinct morphological phenotype of the scirrhous-patterned invasive ductal carcinoma. The expression levels of the MMTV Ptn transgene were also high in clusters of breast cancer cells within the MMTV-PyMT-Ptn breast cancers that morphologically had features of the scirrhous-patterned invasive ductal-like carcinomas but these features were much less well developed (D, FIG. 24) and the levels of expression of the MMTV-Ptn transgene in these morphologically less well developed foci than in the more well developed foci (C, FIG. 24), indicating a dosage effect of the levels of Ptn expression in the development of the scirrhous-patterned invasive ductal carcinoma-like foci in mouse breast cancers. The data support the conclusion that high level expression of Ptn is associated with progression of clones of breast cancer cells to a more aggressive phenotype that correlates with a morphologically distinct subtype of human breast cancers termed scirrhous-patterned invasive ductal carcinoma (Tsuchiya and Li, 2005, supra).

### Increased Matrix Formation in MMTV-PyMT-Ptn Bi-Transgenic Mouse Breast Cancer:

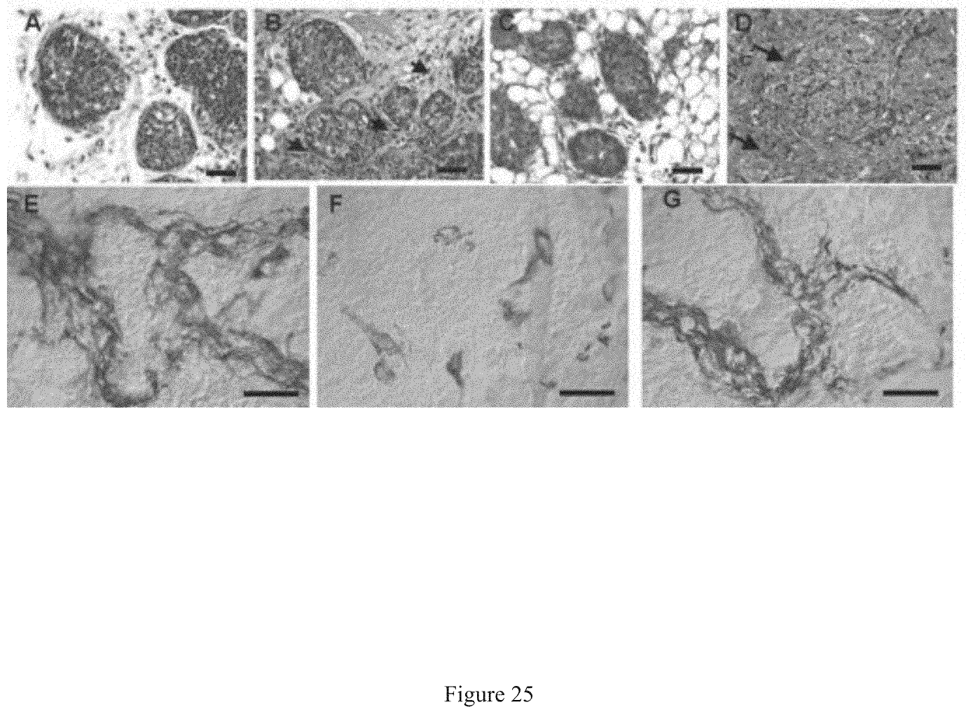

A striking feature of the MMTV-PyMT-Ptn bi-transgenic mouse breast cancers is the remarkable increase in extracellular matrix proteins that circumferentially surround the foci of scirrhous-patterned invasive ductal-like carcinomas that express the highest levels of human Ptn (A and B. FIG. 25). Trichrome stained sections of these breast cancers were used to estimate levels of collagen deposition; a marked increase in collagen fibers surrounding these foci was found; the collagen fibers surrounding these foci were much thicker and more prominent in the breast cancers of MMTV-PyMT-Ptn bi-transgenic mice than that in breast cancers of MMTV-PyMT single transgenic mice (C and D, FIG. 25). Furthermore, when these same sections were stained with resorcin, a component of the Van Gieson staining solution to more specifically mark elastin fibers, a marked increase in mature elastin fibers was also evident in the “fibrillar elastosis” that surrounds the clusters of breast cancer cells in the foci of scirrhous-patterned invasive ductal carcinoma (E, F and G, FIG. 25). In these sections, a striking increase in elastin fibers also was seen to surround the new blood vessels in the same regions of the breast cancers morphologically identified as the scirrhous-patterned invasive ductal-like carcinomas in MMTV-PyMT-Ptn bi-transgenic mice (E and G, FIG. 25). Thus, the increases in collagen and elastin are most prominent in areas of increased matrix protein deposition that surround those foci of aggressive breast cancer in which highest levels of expression of the MMTV-Ptn transgene were found.

### Increased Microvessel Formation in Cancers of MMTV-PyMT-Ptn Bi-Transgenic Mice:

Cryosections from tumors derived from both MMTV-PyMT and MMTV-PyMT-Ptn transgenic mice were used to label blood vessels with anti-CD31 antibodies. The sections were examined to determine the intratumor microvessel density (IMD) as a quantitative index of new blood vessel formation. A nearly two-fold increase was found in the IMD in breast cancers of MMTV-PyMT-Ptn mice compared with the IMD in breast cancers in MMTV-PyMT mice (Left, Table 6). Furthermore, the increase in IMD is higher in areas in close proximity to the foci of scirrhous-patterned invasive ductal-like carcinomas within the highest expression levels of the MMTV-Ptn transgene.

The average microvessel size of the new blood vessels was then compared in breast cancers between MMTV-PyMT single transgenic mice and MMTV-PyMT-Ptn bi-transgenic mice; the average microvessel size in the MMTV-PyMT-Ptn bi-transgenic mice was 659 μm2 and in MMTV-PyMT single transgenic mice it was 393 μm2 (P<0.01, Table 6). Furthermore, the new blood vessels of largest diameter were found in closest proximity to the foci of the scirrhous-patterned invasive ductal-like carcinoma with highest expression levels of the MMTV-Ptn transgene.

Increased Phosphorylation of p44/42 MAPK and Expression of a 46 kDa Isoform of ERα in MMTV-PyMT-Ptn Mouse Breast Cancers:

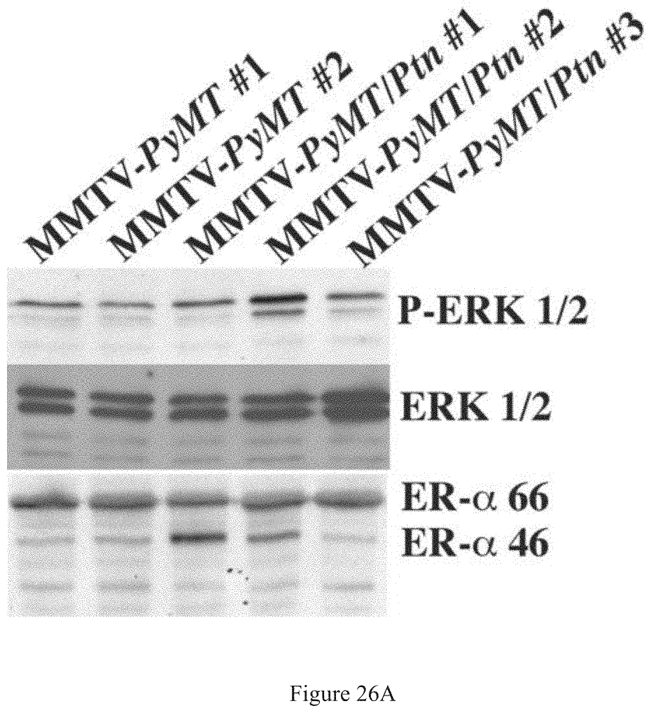

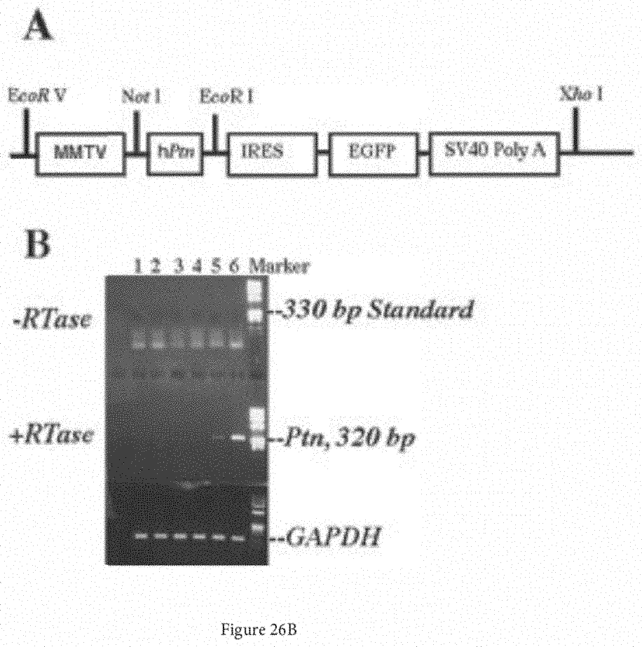

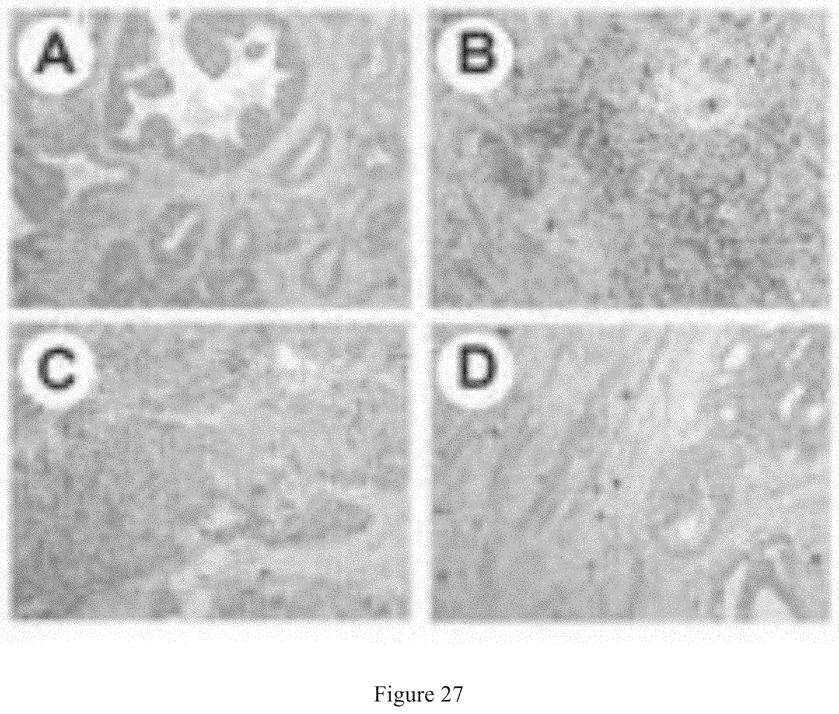

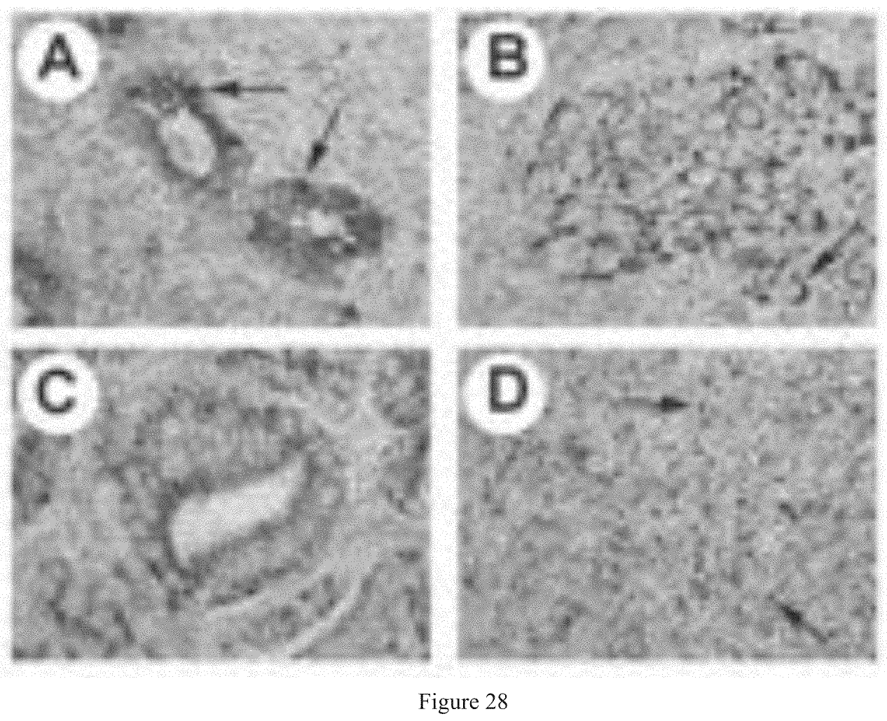

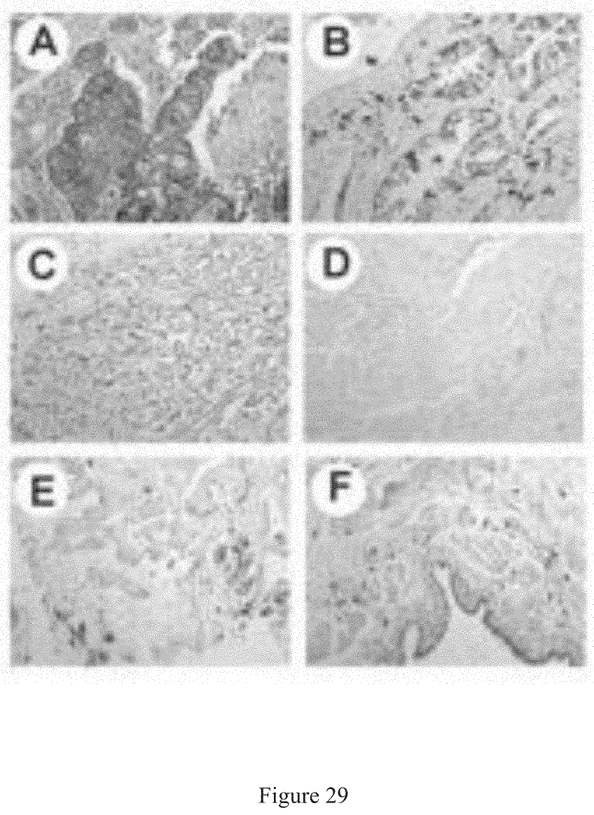

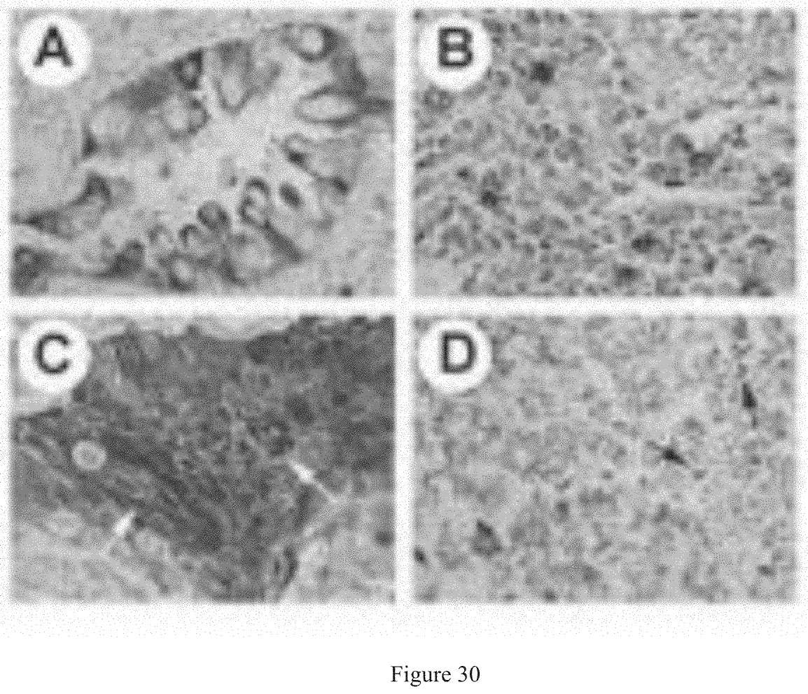

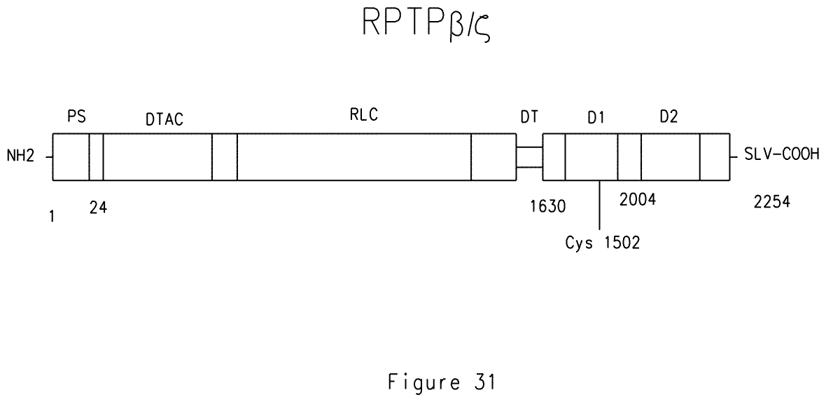

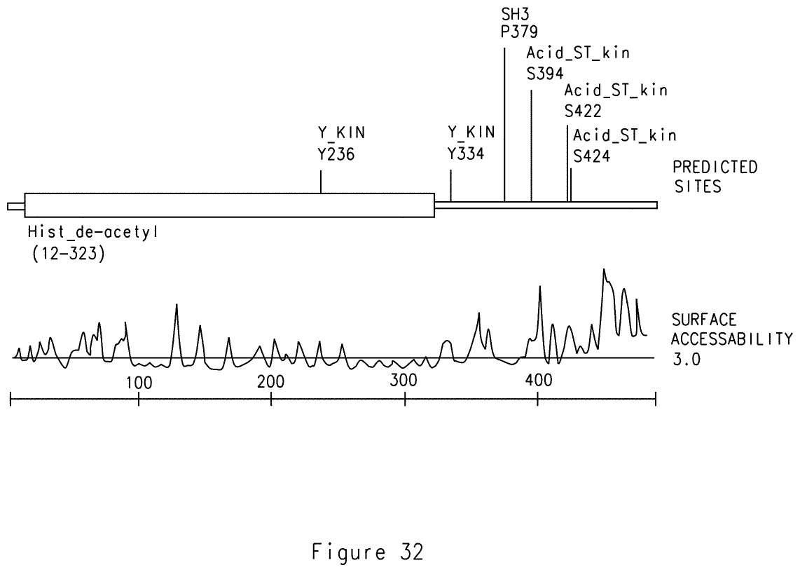

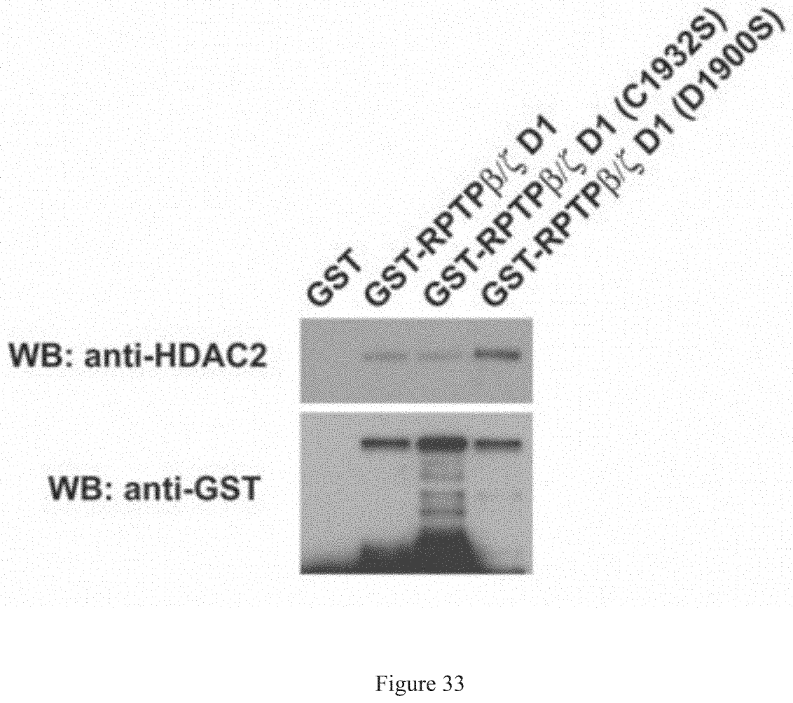

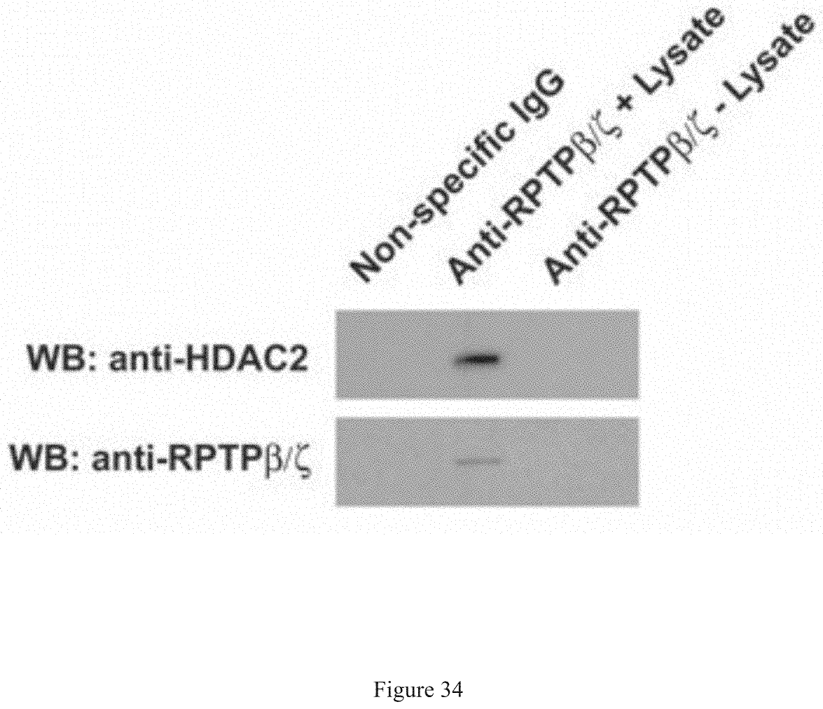

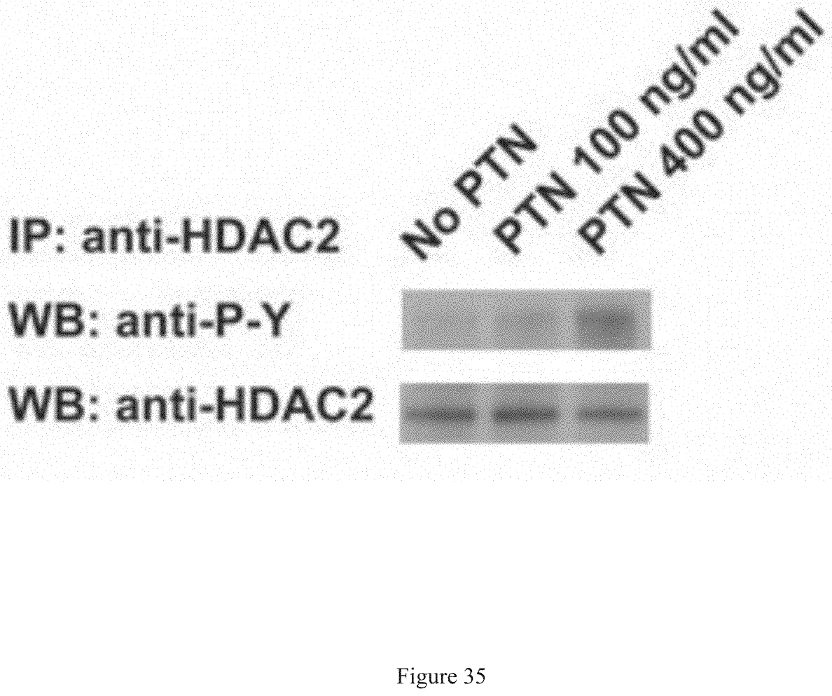

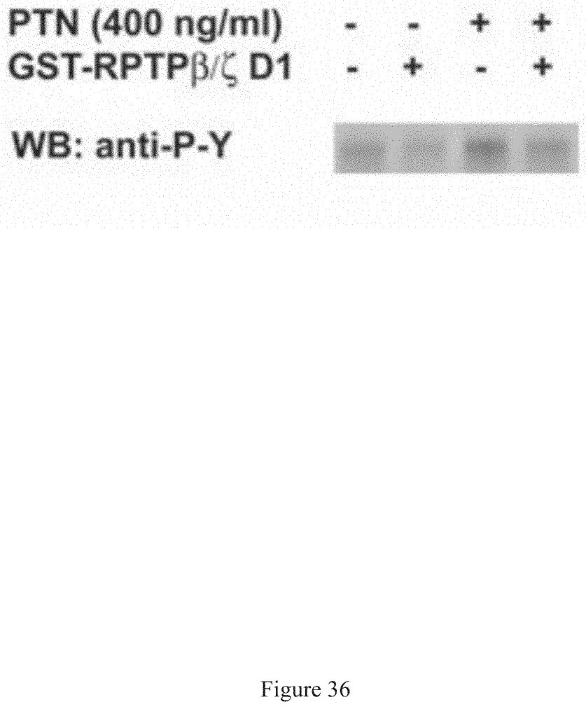

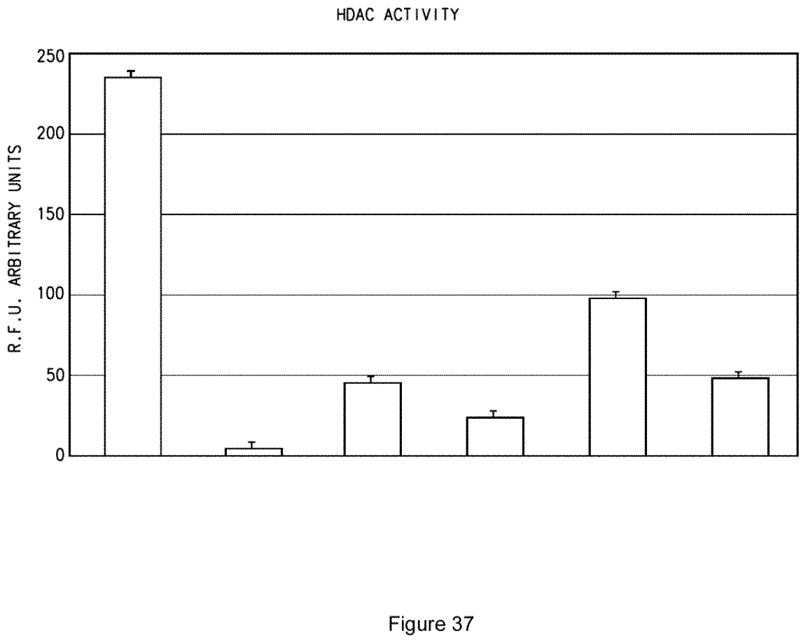

Pleiotrophin stimulates activation of the phosphorylated p44/42 mitogen activated protein kinase (MAPK) signaling pathway (Bernard-Pierrot et al., 2002, J Biol Chem 277, 32071-32077), known to be an effective indicator of cell proliferation and differentiation pathways (Murphy and Blenis, 2006, Trends Biochem Sci 31, 268-275). To test whether activation of the MAPK signaling pathway is higher in MMTV-PyMT-Ptn than in MMTV-PyMT breast cancers, Western blots probed with p44/42 MAPK phosphorylation specific antibodies were compared in lysates in MMTV-PyMT single transgenic and bi-transgenic MMTV-PyMT-Ptn mouse breast cancers. The levels of phosphorylation of p44/42 MAPK were consistently higher in breast cancers from MMTV-PyMT-Ptn single transgenic mice than in breast cancers from MMTV-PyMT single transgenic mice (FIG. 26, top panel) but the levels of P44/42 MAPK protein (control) were essentially equal (FIG. 26, middle panel), thus, the data support the likelihood that the MAPK pathway in breast cancers in MMTV-PyMT/Ptn mice is activated.

The status of the estrogen receptor (ER) a is an important prognostic marker in human breast cancer (Winer et al., 2005, J Clin Oncol 23, 619-629). The full length ERα in both human and mice is 66 kDa. Isoforms of endogenous ERα with different molecular weights of 36, 46 and 80 kDa have been identified in human tissues (Flouriot et al., 2000, Embo J 19, 4688-4700; Pink et al., 1996, Nucleic Acids Res 24, 962-969; Wang et al., 2005, Biochem Biophys Res Commun 336, 1023-1027). Less is known about the variants of ER-a in mice. Antibodies (clone H222) against C-terminus of human estrogen receptor α (ERα) that react with common epitopes in the mouse ER-α were therefore used to measure expression of ER-α(66) in MMTV-PyMT and MMTV-PyMT-Ptn breast cancers; two major bands with molecular weights about 46 and about 66 kDa that correspond in size to the human ERα-46 and ERα-66 were identified in breast cancers from both MMTV-PyMT single transgenic mice and from bi-transgenic MMTV-PyMT-Ptn mice. The levels of expression of the 46 kDa band were strikingly higher in breast cancers of MMTV-PyMT-Ptn bi-transgenic mice (FIG. 26, bottom panel). Differences in levels of the 66 kDa band were not seen nor were differences seen in the less intense band of about 36 kDa observed in breast cancers from MMTV-PyMT-Ptn and MMTV-PyMT mice.

### Discussion

The histological patterns in human breast cancers that result from disruption of the well-organized structures of normal breast tissues are extensively used to classify breast cancers into specific subtypes. These subtypes are important to recognize, since they serve as prognostic markers and to design therapeutic strategies in patients with breast cancer. They are also important since these subtypes are derived from expansion of distinct clones of breast cancer cells whose phenotype is determined by different signaling pathways initiated through sequential mutations during tumor progression. Identifying these mutations and the signaling pathways that lead to these phenotypes thus is a key to identifying new markers and to develop pathway specific therapies for the different forms of breast cancer.

In this study, MMTV-Ptn single transgenic mice were generated to seek the role of inappropriate expression of Ptn driven by MMTV promoter in mouse breast cancer. Expression of Ptn driven by MMTV alone failed to induce breast cancer in mice, establishing that inappropriate expression of Ptn alone is not sufficient to induce breast cancer. In this context, PTN is not an oncogenic protein. Single transgenic mice were then bred with MMTV-PyMT single transgenic mice. MMTV-PyMT mice were selected since breast cancers in MMTV-PyMT mice are heterogeneous in histological phenotype; they contain foci with different histological patterns that mimic different subtypes of human breast cancers; these foci are derived from expansion of individual clones that have constitutive expression of PyMT and additional mutations that activate signaling pathways that cooperate with PyMT-stimulated signaling pathways to initiate breast cancer progression to distinct phenotypes or subtypes in the tumor mass. Since it is important to identify activated pathways that stimulate breast cancer progression, the heterogeneous histological phenotypes in breast cancers of MMTV-PyMT single transgenic mice offered an optimal opportunity to test whether constitutive PTN signaling driven by MMTV-Ptn cooperated with pathways initiated by the PyMT oncogenic protein to stimulate breast cancer progression to a defined phenotype.

The MMTV-PyMT-Ptn bi-transgenic mice developed a nearly 2-fold increase in the size of the breast cancers and a nearly 2-fold increase in foci with the tumor mass that closely resemble the scirrhous-patterned invasive ductal-like carcinoma in humans. The breast cancer cells with this specific phenotype were found to express very high levels of the MMTV-Ptn transgene, thus correlating the high level expression of MMTV-Ptn directly with the scirrhous-patterned invasive ductal-like carcinoma phenotype in mouse breast cancer. Furthermore, less well-developed, but similarly patterned clusters of breast cancer cells were seen that express less, but still high levels of the MMTV-Ptn transgene, raising the possibility a dosage effect of PTN in the development of this phenotype of breast cancers. A striking remodeling of the tumor microenvironment, including a remarkable increase in new collagen and elastin synthesis and increased tumor angiogenesis were features of the breast cancers in MMTV-PyMT-Ptn bi-transgenic mice; these features of MMTV-PyMT-Ptn breast cancers mimic known responses of xenografts of breast cancer cells with inappropriate expression of Ptn (Choudhuri et al., 1997, Cancer Res 57, 1814-1819; Zhang et al., 2006, Biochem Biophys Res Commun 343, 653-658). The data thus support a model in which constitutively activated PTN signaling cooperates with the pathways stimulated by PyMT to promote the more aggressive phenotype of scirrhous-patterned invasive ductal-like carcinoma. These findings thus are likely very important since the model supports PTN is a principle driver, and thus mechanistically important, in different features of this aggressive subtype of breast cancer in MMTV-PyMT-Ptn bi-transgenic mice. Since the scirrhous-patterned invasive ductal carcinoma is among the most aggressive human breast cancers (Tsuchiya and Li, 2005, Med Mol Morphol 38, 216-224), these studies raise the possibility that constitutive PTN signaling likely contributes significantly to this unique breast cancer subtype.

Previous studies also support a significant role of PTN in the pathogenesis of the scirrhous patterned invasive ductal carcinoma; for example, xenografts of (human breast cancer) MCF-7-Ptn cells that express an ectopic Ptn form typical “epithelial islands” when co-cultured with NIH 3T3 cells used as “surrogate” stromal fibroblasts; the epithelial islands in these studies were surrounded by NIH3T3 cells morphologically similar to typical activated stromal fibroblasts interspersed with dense layers of fibrillary matrix proteins identified as collagen and elastin (Zuka, M., Chang, Y., Wang, Z., Berenson, J. R., and Deuel, T. F., Pleiotrophin Secreted from Human Breast Cancer MCF-7-Ptn Cells Activates Stromal Fibroblasts, Induces Epithelial Island Formation and Remodels the Microenvironment; submitted). PTN is known to activate p44/42 MAPK in PTN-stimulated cells. The remarkable increase in both new collagen and elastin synthesis in foci of scirrhous-patterned invasive ductal carcinoma with high-level expression of Ptn correlates directly with the demonstration that PTN stimulates new collagen and new elastin expression in human skin fibroblasts (Ezquerra, supra); collagens and elastin are important in tumor progression and metastasis and, through activation of integrins and “outside-in” signaling, collagens activate the ERK pathway in epithelial cells and function to prevent caspase 8 activation and apoptosis (Hood and Cheresh, 2002, Nat Rev Cancer 2, 91-100). PTN also has been found to be effective angiogenic agent in vivo (Christman et al., 2005, Biochem Biophys Res Commun 332, 1146-1152) and introduction of an ectopic Ptn into malignant cells initiates an “angiogenic switch” in xenografts of Ptn expressing cells in nude mice (Zhang et al., 2006, supra). Furthermore, it has been found that PTN stimulates increased expression of genes of the renin-angiotensin pathway and to increase signaling through the angiotensin II pathway, the renin-angiotensin pathway is known to stimulate angiogenesis (Herradon et al., 2004, Biochem Biophys Res Commun 324, 1041-1047). We think one mechanism through which PTN may stimulate angiogenesis in breast cancers with inappropriate expression of Ptn. In light of our present insight these studies reinforce our view that PTN signaling has a very significant role in the mechanisms that lead to the scirrhous patterned invasive ductal carcinoma in mice.

A potentially interesting but entirely unexplained finding is that Ptn expression in MMTV-PyMT single transgenic mice modifies the expression levels of the different isoforms of the mouse estrogen receptor (ER)a that result from alternative splicing of ER-α66. The human ERα-46 lacks the N-terminal trans-activation domain and thus cannot be activated in the absence of estrogens; these results raise the remote possibility that expression of PTN stimulates the expression of 46 kDa isoform of mouse ERα and potentially affects breast cancer growth in mice.

Commonly cited subtypes of breast cancer which are extensively used to classify breast cancer in humans include the pre-invasive lesions, atypical ductal hyperplasia (ADH) and ductal carcinoma in situ (DCIS), and invasive ductal carcinoma, tubular carcinoma, and invasive lobular carcinoma, although different classifications of breast cancers have been used in different context. Over 70% of human breast cancers are classified as invasive ductal carcinoma, consisting of solid nests or clusters of poorly formed ducts, anastomosing cords, and sheets of breast carcinoma cells. These breast cancers are among the most aggressive human breast cancers (Debnath and Brugge, 2005, Nat Rev Cancer 5, 675-688). Invasive carcinomas have also been divided into three subtypes, papillotubular carcinoma, solid-tubular carcinoma, and scirrhous carcinoma; scirrhous carcinoma is used to more narrowly represent linear or cluster-like invasion of the stroma without forming ducts, with extensive collagen and elastin surrounding the clusters of invasive cells. It accounts for about 16% of breast cancers (Tsuchiya and Li, 2005, supra). The term scirrhous-patterned invasive ductal-like carcinoma appears best suited to the breast cancers described in this study; it fits well with the highly invasive malignant phenotype of breast cancers in MMTV-PyMT-Ptn bi-transgenic mice and importantly, the features of this subtype of breast cancer fit well properties known to be that result from PTN signaling.

Importantly also, within breast cancers, more than one histological pattern often is seen; these different patterns are believed to originate from distinct clones of cells whose phenotype is determined by different signaling pathways activated by different mutations that are superimposed on the breast cancer cell during tumor progression. Little is known about how different mutations dictate the histological patterns of human breast cancers, but the consequences of these mutations are critical to understand, since these consequences are potentially important targets for pathway specific therapy. However, for example, invasive lobular carcinoma is frequently associated with loss of CDH1, the gene which encodes E-cadherin. Comedo dutal carcinoma in situ (DCIS), a type of DCIS, often has amplification of Neu oncogene; whereas, a point mutation in the caveolin 1 gene that alters its subcellular localization is associated with scirrhous breast carcinoma (Lee et al., 2002, Am J Pathol 161, 1357-1369). The identification of these mutations and the breast cancer phenotype associated with them support the view that sequential mutations that occur in progression of breast cancers dictate the phenotypic characteristics of the carcinoma cells and the microenvironment during breast cancers progression and offer new hope for new markers and new targets against which to direct therapy.

### Experimental Procedures:

### Generation of MMTV-Ptn-IRES-GFP Transgenic Mice:

The MMTV LTP promoter and human Ptn cDNA were amplified using PCR from pcDNA3.1-Ptn (constructed in applicant's laboratory). After restriction enzyme digestion, MMTV LTR and Ptn cDNA were inserted into EcoR V, Not I and EcoR I site of pIRES-EGFP (BD Clontech, Mountain View, Calif.). The sequence of the final transgene construction (MMTV-Ptn-IRES-EGFP) was by nucleotide sequencing. Before the microinjection, the transgene construction was freed of all of the bacterial sequences by digestion with EcoR V and Xho I, producing a 4.0-kb linear DNA fragment. The 4.0-kb linear DNA that was injected into the fertilized eggs of FVB mice consisted of the MMTV-LTR, Ptn cDNA, IRES (internal ribosomal entry site), EGFP cDNA, SV40 poly-adenylation sequences. Tail DNA from weaning mice derived from the transgene-injected eggs was analyzed by PCR. The PCR product is about 320 base pairs. The mice were housed in animal facility of The Scripps Research Institute and were maintained according to NIH guideline for the Care and Use of Laboratory Animals.

### Characterization of MMTV-Ptn-IRES-EGFP Transgenic Mice:

The expression of the human Ptn transgene in mammary tissue from both nontransgenic and transgenic mice was analyzed using reverse transcription-polymerase chain reaction (RT-PCR).

### Mice Breeding:

FVB/N-Tg(MMTV-PyVT)634Mul/J transgenic mice were purchased from Jackson Laboratory. They develop spontaneous metastatic mammary carcinomas and have been described in detail by Guy et al (Guy et al., 1992, supra). Female MMTV-PTN mice generated above were bred with male MMTV-PyMT mice to obtain double transgenic mice since female MMTV-PyMT mice did not lactate. The transgenes were passed from generation to generation following a Mendelian inheritance and the resulting genotypes were: wildtype (WT); PTN single transgenic; PyMT single transgenic; and MMTV-PyMT-Ptn bi-transgenic. Transgenic animals were genotyped using PCR analysis of genomic DNA obtained from tails. When possible, litter mates with both genotypes were used in the same experiment.

### Mammary Tumor Detection and Measurement:

Mice were examined daily seeking mammary tumors by eye examination and finger palpation after 4 weeks of age. The tumors that formed fully on their mammary glands were excised and weighed in 13 weeks old animals. Tumor length (L) and width (W) were measured twice a week using calipers. Tumor volume was estimate using the formula (L×W2)/2.

### Metastasis:

The evaluation of metastasis was done according to Williams (Williams et al., 2004). In brief, the left lung obtained from euthanized animals was fixed in 10% formalin and embedded in paraffin. Serial sections were cut at 200 μm interval to cover the whole lung tissue and stained using H&E. The maximum number of tumor metastases in one section was regarded as the metastasis index of that animal.

### Tissue Preparation and Staining:

The tumor tissue excised from euthanized animals were fixed in 10% buffered formalin and embedded in paraffin. The specimens were sectioned, stained with hamatoxylin and eosin (H&E), Trichrome stain for collagen, and Van Gieson stain for elastin and observed under microscope.

### IMD (Intratumoral Microvessel Density):

Tumor tissue was fixed by 4% paraformaldehyde in PBS. After rinsing the tissue at room temperature with PBS, tissue was cryoprotected with 30% sucrose in phosphate buffer at 4° C. overnight, then infiltrated with a mixture of 1 parts 30% sucrose phosphate buffer to 1 part Histo Prep™ Frozen tissue embedding media (Fisher Scientific) for ½ hour at room temperature before infiltration with 100% Histo Prep™ and freezing. For immunofluorescence, Cryosections (7 μm) were blocked with whole goat serum, then incubated with rat monoclonal antibody against mouse CD31 (PECAM-1) (1:100, BD Pharmingen) at room temperature for 2 hours. After rinsing with PBS, cryosections were incubated with FITC-conjugated goat anti-rat IgG (1:200; Santa Cruz Biotechnology Inc, Santa Cruz, Calif.) at room temperature for 1 hour and photographed. Intratumoral microvessel density is a measure of the relative density of tumor angiogenesis in sections of tumor tissue stained with anti-mouse CD31 antibodies. Stained capillaries were counted at ×400 magnification by independent, uninformed investigators and analyzed by statistical analysis of test and control groups. The size of microvessel was measured by SPOT RT 3.4 software (SPOT diagnostic, Sterling Heights, Mich.) using stage micrometer as a calibrator.

### Western Blot Analysis:

Tumor tissues were rinsed and then lysed in T-PER tissue protein extraction (Pierce, Rockford, Ill.) supplemented with protease inhibitor cocktail (Roche Diagnostics) for 30 min on ice. After removal of cell debris at 12,000 rpm, the protein concentration of the supernatants was measured using the BCA method (Pierce, Rockford, Ill.). Proteins were separated on SDS-PAGE, transferred to nitrocellulose membrane and probed with appropriate antibodies. Anti-PTN antibodies were purchased from R&D system (Minneapolis, Minn.). Anti-MAPK antibodies were purchased from Cell Signaling (Beverly, Mass.) and ER antibodies (H222) were from Research Diagnostics Inc (Concord, Mass.). HRP-conjugated secondary antibodies were obtained from Santa Cruz Biotechnology (Santa Cruz, Calif.).

### In Situ Hybridization:

For RNA in situ hybridization, the human PTN cDNA sequence was subcloned into pCRII (Invitrogen, Calif.), the plasmids were linearized, and the sense and antisense riboprobes were synthesized from the SP6 or T7 transcription sites with a digoxigenin (DIG)-RNA Transcription Kit (Roche Diagnostics, Indianapolis, Ind.). Formalin-fixed, deparaffinized sections were incubated for 10 min at 37° C. with 20 μg/mL proteinase K in 10 mM Tris-EDTA (ethylenediamine tetra-acetic acid) buffer to assess the effects of proteolytic treatment, then treated with 0.2 M HCl for 10 min. The sections were dehydrated rapidly in increasing concentrations of ethanol and air-dried. The DIG-labeled PTN probes, both sense and antisense, were diluted 1:10 with hybridization buffer (Bioprobe System, Montrevil, France). The sections were incubated with a hybridization solution in a moist chamber at 45° C. for 16 h. The sections were then washed sequentially in 2×SSC containing 50% formamide for 30 min at 50° C., in 2×SSC 20 min at 50° C., and twice in 0.2×SSC for 20 min at 50° C. After brief wash in Tris-buffered saline (100 mM Tris-HCL, pH 7.0, 150 mM NaCl), the sections were incubated with a 1.5% blocking reagent (Boehringer Mannheim, Mannheim, Germany) for 1 h to reduce the non-specific background, then incubated with a 1:500 dilution of alkaline phosphatase-conjugated sheep anti-DIG antibody (Boehringer Mannheim) at room temperature for one hour. The sections were washed twice in Tris-buffered saline for 15 min each, then incubated with equalization buffer (100 mM Tris-HCL, pH 9.5, 100 mM NaCl, 50 mM MgCl2) for 3 min. Color development was achieved by adding a freshly prepared substrate solution containing nitroblue tetrazolium salt (450 μg/mL) and 5-bromo-4-chloro-3-indolyl phosphate toluidinium salt (175 μg/mL) in equilization buffer to the slides for one hour. Negative controls of in situ hybridization included pretreatment of the sections with 100 μg/mL RNase A or with no probe, and also hybridization of the sections with the sense probe.

### Statistics:

Results were presented as mean±SEM. Student test was used to determine the statistical significance of tumor onset time, tumor size and scirrhous area of tumors. Unpaired t-test was used to determine the statistical significance of the differences in number of lung metastatic foci.

TABLE 3
Tumor onset, tumor burden and lung metastasis in
MMTV-PyMT and MMTV-PyMT-Ptn bi-transgenic mice.

| | Tumor | Tumor burden | Lung |
|---|---|---|---|
| Genotype | onset time | at 13 week age | metastasis site |
| MMTV-PyMT | 55.7 ± 5.2 | 4.94 ± 1.59 | 3.17 ± 4.30 |
| | (n = 16) | (n = 12) | (N = 18) |
| MMTV-PyMT-Ptn | 53.4 ± 3.6 | 4.12 ± 1.34 | 1.88 ± 3.04 |
| | (n = 14) | (n = 10) | (N = 17) * |

* P = 0.318, unpaired t-test

TABLE 4
Average tumor size of MMTV-PyMT and
MMTV-PyMT-Ptn bi-transgenic mice.

| | | | | Tumor | Average tumor volume |
|---|---|---|---|---|---|
| Mouse | Genotype | Sex | Week | Number | (mm³) |
| D11531 | PyMT+ | F | 13 | 8 | 373.12 |
| D11532 | PyMT+ | F | 13 | 12 | 353.45 |
| D11528 | PyMT+/Ptn+ | F | 13 | 10 | 250.42 |
| D11533 | PyMT+/Ptn+ | F | 13 | 7 | 797.12 * |
| D11534 | PyMT+/Ptn+ | F | 13 | 10 | 753.33 * |

These mice are from the same litter.
* P > 0.05, this result thus is not statistically significant.

TABLE 5
Percentage of areas of scirrhous patterned carcinoma of tumors
from MMTV-PyMT and MMTV-PyMT-Ptn bi-transgenic mice (H&E).

| | Clustered area | Non-clustered area |
|---|---|---|
| MMTV-PyMT (N = 22, %) | 22.5 ± 9.1 | 19.2 ± 15.8 |
| MMTV-PyMT-Ptn (N = 25, %) | 38.9 ± 5.0 * | 16.8 ± 16.6 |

*P < 0.05

TABLE 6
Intratumor microvessel density (IMD) and vessel size
of tumors from MMTV-PyMT and MMTV-PyMT-Ptn bi-transgenic
mouse (immunofluorescence against mouse CD31).

| | IMD | Microvessel size |
|---|---|---|
| | (counts/mm) | (μm²) |
| MMTV-PyMT (N = 22) | 18.83 ± 10.2 | 393 ± 109 |
| MMTV-PyMT- | 27.14 ± 13.5 | 658 ± 272 * |

* P < 0.01

### Example 5

### Blockade of Endogenous Pleiotrophin Signaling Prevents Breast Cancer Progression to More Aggressive Phenotypes

We show elsewhere herein that constitutive expression of pleiotrophin (PTN, Ptn) introduced by mouse mammary tumor virus (MMTV) into polyoma virus middle T (PyMT)-Ptn bi-transgenic mice stimulates progression of breast cancers to the aggressive “scirrhous” carcinoma phenotype through remodeling of the tumor microenvironment, marked increases in new collagens and elastin, in new blood vessels, and increases in stromal fibroblasts, which are seen to associate with the more aggressive breast cancer phenotypes. Surprisingly, it was found that MMTV-PyMT mouse breast cancer cells expressed significant levels of endogenous Ptn, indicating the endogenous Ptn also contributed to progression of breast cancer. In the present study, a dominant negative Ptn driven by MMTV (MMTV-PTN 1-40) was introduced into MMTV-PyMT mice; foci of scirrhous carcinoma were not found in breast cancers from MMTV-PyMT-PTN 1-40 mice, indicating constitutive Ptn expression is required for this aggressive breast cancer phenotype in mice. Furthermore, progression of early stage to late stage carcinomas also was delayed. Tumor angiogenesis was significantly lacking and the angiogenic foci were not associated with the foci of breast cancer. There was a marked reduction of stromal fibroblasts in MMTV-PyMT-PTN 1-40 mouse breast cancers and new collagens and elastin were limited and not associated with the cancer cells. The data in the instant Example thus support the hypothesis that constitutive Ptn expression stimulates progression of breast cancers to a more aggressive breast cancer phenotype. We hypothesize that deregulated Ptn expression is a critical determinant of the more aggressive phenotype in mouse breast cancer.

Different transgenic mouse models with targeted dominant oncogenes have been used to effectively demonstrate incremental steps in tumor progression and, as the cancers progress, to demonstrate that the more malignant foci arise within the foci of lesser malignancy to then assume increasingly more of the tumor mass (Hanahan D, Weinberg R A, Cell, 2000; 100:57-70). Identification of the different stages in malignant cancers and the relative areas they occupy within these malignant cancers thus is a reliable measure of tumor progression; this increase is of critical importance in evaluating human malignancies and an essential component of “staging” of malignant cancers to assess prognosis and to design effective therapies. Thus, the need to identify the mutations that initiate switches is a high priority.

Pleiotrophin (PTN the protein, Ptn the gene) is a 136 amino acid heparin-binding cytokine. Pleiotrophin activates multiple signaling pathways that induce new phenotypes in stimulated cells that are phenotypes characteristic of highly malignant cells (Perez-Pinera, 2006, supra). Among these phenotypes are loss of cell-cell adhesion, extensive remodeling of the cytoskeleton, an epithelial mesenchymal transition (EMT) and stimulation of tumor angiogenesis. Pleiotrophin furthermore stimulates striking increases in new collagen and elastin, upregulates MMP-9, known to be critical mediator of tumor progression, and protein kinase C ∝, which is a marker with MMP-9 of malignant breast cancers.

Pleiotrophin is expressed in breast cancers, and it is expressed in cell lines derived from human breast cancers. Interruption of PTN signaling with a dominant negative PTN in human breast cancer cells prevents breast cancer cells from effectively establishing implants in nude mice, and thus, it also prevents exit from dormancy (Zhang et al., 1997, supra). It furthermore reverses the transformed phenotype of human breast cancer cells along with the extensive tumor angiogenesis and striking remodeling of the microenvironment.

The potential of PTN signaling to stimulate breast cancer progression was directly demonstrated in an Example above of the mouse mammary tumor virus (MMTV) promoter-driven polyoma virus middle T (PyMT) mouse breast cancer model. This model was selected since four distinctly identifiable stages of breast cancer progression are found; these stages include ductal hyperplasia, adenoma/mammary intraepithelial neoplasia (MIN), early carcinoma, and later carcinoma. MMTV-driven Ptn single transgenic mice were then generated and bred with MMTV-PyMT mice. MMTV-PyMT-Ptn bi-transgenic mouse breast cancers developed a nearly two-fold increase in the area of the breast cancers that was occupied by “later carcinoma”. Thus, Ptn expression stimulated tumor progression. Surprisingly, there was a nearly two fold increase within the foci of later carcinoma in foci occupied by malignant cells with the histological phenotype of “scirrhous” carcinoma in humans (Chang Y, et al., Proc Natl Acad Sci USA, 2007;104:10888-93). There was also extensive remodeling of the microenvironment associated with the foci of scirrhous carcinoma, striking increases in mouse procollagens and in tropoelastin, significant increases in new blood vessels of increased size, and increases in numbers of “activated” fibroblasts that were found to surround the foci of the most aggressive breast cancer. Each of these findings was found to directly correlate with highest level expression of Ptn.

A surprising finding in the study of MMTV-PyMT-Ptn mice was the PyMT mouse breast cancer cells themselves inappropriately expressed Ptn. In the present Example. we determine whether inappropriate expression of Ptn in MMTV-PyMT mouse breast cancers contributed to the aggression phenotype of MMTV-PyMT breast cancers. To do so, transgenic mice were generated in which a dominant negative Ptn driven by MMTV was introduced (MMTV-PTN 1-40 mice). MMTV-PTN 1-40 mice were then bred with MMTV-PyMT mice to generate mice that lacked effective endogenous PTN signaling in breast epithelial cells.

The present Example demonstrates that dominant negative PTN effectively retards the progression of breast cancers in MMTV-PyMT mice; the results support the rapid and aggressive growth of MMTV-PyMT mouse breast cancers may be in part due to deregulated expression of Ptn.

### Experimental Procedures:

### Generation of MMTV-PTN1-40-IRES-GFP Transgenic Mice:

The MMTV LTP promoter and human cDNA encoding PTN 1-40 (residue −32 to residue 40) was amplified using PCR from pcDNA3.1-Ptn (constructed by our laboratory). After restriction enzyme digestion, MMTV LTR and Ptn 1-40 cDNA were inserted into EcoR V, Not I and EcoR I site of pIRES-EGFP (BD clontech). The sequence of the final transgene construction (MMTV-PTN-IRES-EGFP) was verified by nucleotide sequencing. The transgene construction was freed of the bacterial sequences by digestion with EcoR V and Xho I, producing a 4.0-kb linear DNA fragment. The 4.0-kb linear DNA that was injected into the fertilized eggs of FVB mice consisted of the MMTV-LTR, PTN 1-40 cDNA, IRES (internal ribosomal entry site), EGFP cDNA, SV40 poly-adenylation sequences. Tail DNA from weaning mice derived from the transgene-injected eggs was analyzed by PCR. The mice were housed in animal facility of The Scripps Research Institute and were maintained according to NIH guidelines for the Care and Use of Laboratory Animals.

### Mice Breeding:

FVB/N-Tg(MMTV-PyVT)634Mul/J transgenic mice were purchased from Jackson laboratory. They develop spontaneous metastatic mammary carcinomas and have been described in before. Female MMTV-PTN 1-40 mice generated above were bred with male MMTV-PyMT mice to obtain double transgenic mice since female MMTV-PyMT mice did not lactate. Transgenic animals were genotyped using PCR analysis of genomic DNA obtained from tails. When possible, litter mates with both genotypes were used in the same experiment.

### Mammary Tumor Detection and Measurement:

Mice were examined daily seeking mammary tumors by eye examination and finger palpation after 5 weeks of age. The tumors fully formed on the mammary glands were excised and weighed in 12-14 weeks old mice depending on the tumor size in bi-transgenic mice. Tumor length (L) and width (W) were measured 3 times a week using calipers. Tumor volume was estimated using the formula (L×W2)/2. Total wet tumor weight was weighed by scale when the tumors were excised from sacrificed mice.

### Tissue Preparation and Staining:

The tumor tissue excised from euthanized animals was fixed in 10% buffered formalin and embedded in paraffin. The specimens were sectioned, stained with H&E, and Masson trichrome stain for collagen fibers.

### IMD (Intratumoral Microvessel Density):

Tumor tissue was fixed by 4% paraformaldehyde in phosphate-buffered saline (PBS). After PBS rinse, the tissues were cryoprotected with 30% sucrose in PBS at 40 C overnight, then infiltrated with a mixture of 1 parts 30% sucrose phosphate buffer to 1 part Histo Prep™ Frozen tissue embedding media (Fisher Scientific) for ½ hour at room temperature before infiltration with 100% Histo Prep™ and freezing. Cryosections (7 mm) were cut and immunohistochemistry was performed using rat anti-mouse CD31 (PECAM-1. BD Pharmingen) antibodies. IMD (number of vessels/mm2) was obtained by counting CD31+ vessels in 10 random fields at ×200 magnification.

### Western Blot Analysis:

Tumor tissues were rinsed and then lysed in T-PER tissue protein extraction (Pierce), both solutions were supplemented with protease inhibitor cocktail (Roche Diagnostics). HRP-conjugated secondary antibodies are obtained from Santa Cruz Biotechnology (Santa Cruz, Calif.).

### RNA Extraction:

Three tumors from a mouse were minced together in liquid nitrogen and total RNAs were extracted by RNAeasy kit (Qiagen). Three mice from each genotype, MMTV-PyMT, MMTV-PyMT-Ptn and MMTV-PyMT-PTN 1-40 were used. RNA quality was monitored by measuring the ratio of A260/280 and agarose gels.

### Realtime RT-PCR:

Reverse transcription was done using 2 mg total RNA in the presence of 50 ng random hexamers using Ominscipt kit (Qiagen) following manufacturer's recommendations. Real time PCR was done using ICycler (BIORAD). The amplification conditions were: one cycle of 95° C.°, 3 min followed by 40 cycles of 95 C°, 30 sec and 60 C°, 15 sec. GAPDH was used as internal control. The fold changes relative to GAPDH was calculated using the formula: Fold Change=2 L (Ct Target1-CtTarget2)/2 L (Ct GAPDH1-CtGAPDH2) Where: Ct is the threshold of cycle number. Target is the gene measured. Number 1 and 2 represent different samples.

### Statistics:

Results were presented as mean±SEM. Student T-test was used to determine the statistical significance. Significant difference was accepted when the P value was less than 0.05 and very significant difference when p value was less than 0.001.

### Results:

### Generation of MMTV-PyMT-PTN 1-40 Mice:

Four identifiable stages of tumor progression were previously identified within a single primary focus of breast cancer in MMTV-PyMT mice in a comprehensive study of Lin et al. (Am J Pathol 2003;163:2113-26). These stages include ductal hyperplasia, adenoma/mammary intraepithelial neoplasia (MIN), early carcinomas, and later carcinoma; these stages closely recapitulate the multi-steps characteristic of human breast cancer progression (Lin et al., Am J Pathol 2003;163:2113-26). Transgenic mice expressing the dominant negative PTN mutant (PTN 1-40) driven by the MMTV promoter (MMTV-PTN 1-40 mice) were first generated. The dominant negative Ptn gene encodes the endogenous signal peptide and PTN residues 1-40; it functions as a dominant negative by forming mixed disulfide linked heterodimers (Zhang, et al., 1997, supra). Expression of PTN 1-40 was readily demonstrated in mammary glands of female MMTV-PyMT-PTN 1-40 mice (data not shown). MMTV-PTN 1-40 mice have normal numbers of pups. Neither abnormality of mammary gland development nor of lactation were found. MMTV-PTN 1-40 mice were then bred with MMTV-PyMT mice to generate MMTV-PyMT-PTN 1-40 (bi-transgenic) mice. The breast cancers were studied in comparison with breast cancers from MMTV-PyMT and MMTV-PyMT-Ptn mice.

### Expression of PTN 1-40 Retards Progression of Breast Cancers in MMTV-PyMT Mice:

In the present study of MMTV-PyMT-PTN 1-40 mice, foci of the very aggressive scirrhous carcinoma were not seen in any of the MMTV-PyMT-PTN 1-40 bi-transgenic mouse breast cancers analyzed. FIG. 40, C demonstrates the “later carcinomas” in MMTV-PyMT-PTN 1-40 mice. It represents the most aggressive stage of breast cancer in the MMTV-PyMT-PTN 1-40 mouse and contrasts strikingly with the more aggressive stages found in MMTV-PyMT mice (FIG. 40, A) and in MMTV-PyMT-Ptn mice. In the earlier study cited above (Chang et al., 2007, supra), the area occupied by scirrhous carcinoma in MMTV-PyMT-Ptn bi-transgenic mice was 39%; the area occupied by scirrhous carcinoma in MMTV-PyMT single transgenic mice was 22% (FIGS. 40, A and B). The data thus demonstrated the dominant negative PTN and loss of the PTN signal in breast epithelial cells in MMTV-PyMT-PTN 1-40 mice correlates directly with the failure of the breast cancers to progress to scirrhous breast cancer. The data support PTN signaling stimulates breast cancer cells to the highly aggressive scirrhous phenotype.

The percentage of the areas occupying breast cancers for each of the subtypes of breast cancer relative to the area occupied by the breast cancer itself in MMTV-PyMT and MMTV-PyMT-PTN 1-40 mice were then compared (Table 7). Examples of the scirrhous phenotype of aggressive breast cancer is seen in FIG. 40; FIG. 40A is an example of the scirrhous phenotype in breast cancers in MMTV-PyMT mice. The scirrhous patterns of breast cancer cells are surrounded by dense layers of collagen- and elastin-like fibrils interspersed with fibroblasts that appear “activated.” FIG. 40B is an example of a scirrhous patterned focus of breast cancer from MMTV-PyMT-Ptn mice; in this case, the fibrillar proteins surrounding the focus of scirrhous breast cancer are denser and there are increased activated stromal fibroblasts. FIG. 40C depicts breast cancer foci from MMTV-PyMT-PTN 1-40 mice taken from an area of later carcinoma. FIG. 40C establishes these foci are less aggressive in appearance and essentially devoid of activated fibroblasts and the fibrillar proteins identified in more aggressive scirrhous carcinoma as collagens and elastin. The percentage area occupied by “later carcinoma” in sections of MMTV-PyMT-PTN 1-40 mouse breast cancers is about 45%; whereas, the percentage area occupied by later carcinoma in breast cancers in MMTV-PyMT mice is about 78% (P=0.024, Table 7). The percentage areas occupied by early carcinoma in MMTV-PyMT-PTN 1-40 mouse breast cancers is 41% compared to 20% in MMTV-PyMT mouse breast cancers (p=0.045). The percentage areas of adenoma/MIN in MMTV-PyMT-PTN 1-40 mouse breast cancers is 12% compared with 2% in MMTV-PyMT mouse breast cancers (p=0.017). The data thus support the hypothesis that endogenous Ptn in MMTV-PyMT breast cancers induces significant levels of MMTV-PyMT breast cancer progression from adenoma/MIN to early carcinoma, from early carcinoma into later carcinoma, and within later carcinoma, to the highly aggressive scirrhous phenotype; a finding supported by the even higher fold in scirrhous carcinoma in MMTV-PyMT-Ptn breast cancers. The data also document the dominant negative PTN 1-40 in breast cancer epithelial cells in MMTV-PyMT-PTN 1-40 mice effectively retards progression of breast cancers in MMTV-PyMT-PTN 1-40 mice and thus correlate expression levels of Ptn in breast epithelial cells with the rate of progression of MMTV-PyMT mouse breast cancers. It appears that the rate of progression of breast cancer in MMTV-PyMT mice to the more malignant phenotype is dose dependent. Breast cancers from MMTV-PyMT-PTN 1-40 mice have markedly reduced activated stromal fibroblasts.

In the present Example, there was a marked reduction of morphologically identified “activated” fibroblasts in MMTV-PyMT-PTN 1-40 breast cancers compared with the abundant numbers of fibroblasts that surround foci of the aggressive breast cancers in MMTV-PyMT-Ptn and MMTV-PyMT mice. The MMTV-PyMT breast cancers with more limited expression of Ptn have a more limited but still significant number of activated stromal fibroblasts, seen also to surround the foci of breast cancer, whereas in breast cancers of MMTV-PyMT-PTN 1-40 mice, the fibroblasts were found largely scattered throughout. Surprisingly, these fibroblasts in breast cancers from MMTV-PyMT-PTN 1-40 mice express Ptn (FIG. 41).

Breast cancers from MMTV-PyMT-PTN 1-40 mice express only low levels of collagens and elastin. In the previous study cited above (Chang et al., 2007, supra), remarkable increases in expression of tropoelastin, protocollagen Iα2, protocollagen IVa5 and protocollagen XIa1 were found in breast cancers of MMTV-PyMT-Ptn mice; procollagen Ia2 was found about 34 fold increased when compared with MMTV-PyMT mice. Striking increases in procollagens IVa5 (about 53 fold), XIa1 (about 54 fold), and tropoelastin (about 4 fold) also were found in MMTV-PyMT-Ptn breast cancers compared with MMTV-PyMT breast cancers. Striking increases in deposition of new collagens and elastin in MMTV-PyMT-Ptn breast cancers were localized surrounding the foci of the most aggressive breast cancers. In this study of MMTV-PyMT-PTN 1-40 mouse breast cancers, the dense layers of collagen and elastin interspersed with high density characteristic of MMTV-PyMT-Ptn breast cancers were not found. RT-PCR was then used to measure expression levels of different protocollagen and tropoelastin mRNAs in breast cancers from MMTV-PyMT and MMTV-PyMT-PTN 1-40 mice. The data obtained were then compared with the data obtained from the previous study (FIG. 42). The levels of tropoelastin, protocollagen la1, protocollagen Iα2, protocollagen IVa5 and protocollagen XIa1 mRNAs were not statistically different when compared with breast cancers from MMTV-PyMT-PTN 1-40 mice and MMTV-PyMT mice (FIG. 42). They were markedly reduced in comparison to the levels of procollagens and tropoelastin that were found previously in breast cancers in MMTV-PyMT-Ptn mice, which have the highest level expression of Ptn in the breast cancer epithelial cells themselves, supporting again PTN may be singularly important in progression of breast cancers to the scirrhous phenotype and the possibility Ptn induced new collagens and elastin progression to scirrhous carcinoma.

### Tumor Angiogenesis is Reduced in MMTV-PyMT-PTN 1-40 Mouse Breast Cancers:

Constitutive expression of Ptn from malignant cells is known to stimulate tumor angiogenesis. Furthermore, constitutive expression of Ptn in MMTV-PyMT-Ptn mice was associated with a nearly two-fold increase of tumor angiogenesis in MMTV-PyMT-Ptn breast cancers and the sites of tumor angiogenesis in MMTV-PyMT-Ptn mice correlated directly with sites of highest level of Ptn expression. The size of the new blood vessels also was markedly increased, and elastin staining within the walls of the new blood vessels was prominent.

Cryosections from both MMTV-PyMT and MMTV-PyMT-PTN 1-40 mouse breast cancers were therefore prepared and the numbers of new blood vessels within the breast cancers compared. FIG. 43 demonstrates the intratumor microvessel density in the MMTV-PyMT-PTN 1-40 mouse breast cancers (B) was significantly reduced compared with the IMD in MMTV-PyMT mouse breast cancers (A). Table 8 shows the average IMD in MMTV-PyMT mouse breast cancers is about 30 blood vessels/mm2 whereas the average IMD in MMTV-PyMT-PTN 1-40 mouse breast cancers is about 18 blood vessels/mm2 (P<0.05), confirming new blood vessels in the breast cancers of the MMTV-PyMT-PTN 1-40 mice are significantly reduced. The CD31 new vessels were localized together with the more malignant foci in the MMTV-PyMT breast cancers at sites where Ptn expression is highest.

These data support previous studies that demonstrate tumor angiogenesis is markedly reduced in human breast cancer cells and other cancer cells that express dominant negative PTN (Zhang et al., 1997, supra). The data are compatible with the hypothesis that PTN secretion from cancer cells is a potent stimulus to new blood vessel formation in the progression of malignant cancers.

### Discussion

Different transgenic mouse models have established that malignant cancers progress through discrete, readily identifiable stages from benign hyperplasia to highly malignant invasive cancer phenotypes; characteristically, the malignant cells themselves have a more aggressive and invasive phenotype. Furthermore, they are surrounded by extensive remodeling of the microenvironment and abundant tumor angiogenesis leading to a high incidence of metastasis (Hanahan D, et al., 2000, supra; Perez-Pinera et al., 2007, supra). In the different studies, it is significant that the more malignant foci arose within areas of less aggressive cancers, supporting the conclusion that additional mutation(s) occur in the less malignant cells that favor the growth of the malignant cells; through clonal expansion of the more malignant foci within the areas of lesser malignancy, the areas occupied by the more malignant cells come to dominate the tumor mass and to make the tumors themselves more highly malignant. Measurement of the areas occupied by the more highly malignant foci thus is an important and sensitive tool to test tumor progression. The studies support the critical need to identify the mutations and mechanisms that stimulate the transition of malignant cells to stages of higher malignancy. Identifying these mutations and mechanisms not only leads to better understanding of highly malignant cancers, it leads to the identification of targets and target specific therapies needed to effectively treat malignant cancers. In the instant Example, levels of constitutive PTN signaling were correlated with areas of foci of different stages of tumor progression to support the conclusion the levels of Ptn expression correlate directly with increased malignant stages of tumor progression. It is concluded PTN is a major factor stimulating breast cancer progression in MMTV-PyMT mice.

These studies support several intriguing possibilities and/or likelihoods. The studies are compatible with the hypothesis that PTN stimulates tumor progression through multiple mechanisms. It is important that PTN does not stimulate progression of cancer cells through secretion of PTN alone. PTN-stimulated cells activate anaplastic lymphoma kinase (ALK), stimulate tyrosine phosphorylation of b-catenin, disassociate b-catenin from cadherin and adherent junction complexes, induce loss of cell-cell adhesion, and induce an EMT. PTN furthermore stimulates transport of b-catenin to nuclei and induces transcription of genes known to transduce progression of malignant cells (see Example above). However, secretion of PTN also acts to remodel the microenvironment through increased collagen and elastin synthesis and to induce tumor angiogenesis. The present studies also support the importance of “activated” fibroblasts that were seen to surround the foci of the scirrhous breast cancers.

Of major significance, the foci of scirrhous carcinoma were found to express highest level expression of Ptn gene; furthermore, highest level Ptn expression was associated with the highest density of tumor angiogenesis, new collagen, and new elastin deposition and with activation fibroblasts. Thus, the data support our hypothesis that constitutive expression of Ptn underlies the pathogenesis of the scirrhous subtype of invasive ductal carcinoma.

The importance of the epithelial cancer cell-stromal fibroblast interactions is well established (Allinen M et al, Cancer Cell 2004; 6:17-32; Orimo A et al., Cell, 2005; 121:335-48). However, only limited progress has been made in identifying the factors that activate stromal cells to initiate cross talk and tumor progression. The instant Example and to an extent our previous study (Chang et al., 2007, supra) identify PTN as a factor that activates stromal fibroblasts to induce many of the key features in breast cancer progression; it induces the characteristic morphological features and different biomarkers such as expression of the extracellular matrix proteins, including collagens, elastin, and matrix metalloproteinases (MMPs). Our work demonstrates that activated stromal cells are central in the models used in this study; they are responsible for the synthesis, deposition and remodeling of the extracellular matrix (ECM) proteins including collagen and elastin. Stromal cells release factors that stimulate the growth and malignant phenotype of the carcinoma cells themselves (Kalluri R., Nat Rev Cancer 2003; 3:422-33). These studies indicate PTN secreted from the breast cancer cells is the mechanism of stromal cell activation; the earlier studies (Chang et al, 2007, supra) also identify PTN alone is sufficient to stimulate in activated fibroblasts many of the critical signaling pathways needed to aggressively promote breast cancer progression.

Pleiotrophin is known to stimulate new blood vessel formation and tumor angiogenesis. Pleiotrophin is known to stimulate new collagen of different subtypes and new elastin synthesis; collagen fragments are known to stimulate growth of carcinoma cells and to upregulate anti-apoptotic pathways, thus supporting the growth of the carcinoma cells (Ruhl M et al., J Biol Chem 1999; 274:34361-8). Through activation of integrins and “outside-in” signaling, both elastin and collagens activate the ERK pathway in epithelial cells; they function to prevent caspase 8 activation and apoptosis (Sanders M A, et al., J Biol Chem 2000; 275:38040-7); the data support our thought the striking increase in extracellular matrix proteins is a very important mechanism that contributes to progression of foci of scirrhous carcinoma in MMTV-PyMT-Ptn breast cancers. In earlier studies (Chang et al., 2007, supra), secretion of PTN also was found to up-regulate two well-recognized markers of aggressive breast cancers, PKCd, and MMP-9, remarkably, both in breast cancer cells and in stromal fibroblasts.

Table 7. Distribution of different histological phenotypes of breast cancers in 13-week age MMTV-PyMT and MMTV-PyMT-PTN 1-40 mice.

Table 8. Intratumor microvessel density of breast cancers in 13-weekage MMTV-PyMT and MMTV-PyMT-PTN1-40 mice.

1. A method for treating cancer, reducing risk of developing cancer, reducing cancer cell proliferation or reversing tumor growth, in a subject comprising administering a medicament including an effective amount of an antibody against PTN, a fragment thereof, negative PTN, decoy RPTP β/ζ, or combinations thereof to a subject in need thereof.

2. The method of claim 1 comprising:
determining the association or dissociation state of the β-catenin/E-cadherin complex in a cell of the subject, wherein the amount of the medicament is increased if the β-catenin/E-cadherin complex is substantially dissociated, or β-catenin is substantially in the form of β-catenin-phosphotyrosine-333.

3. The method of claim 1 further comprising:
a) selecting the medicament on the basis of the medicament being capable of substantially increasing the association of the β-catenin-tyrosine-333 with E-cadherin-phosphoserine-692 to form β-catenin/E-cadherin complex in a cell of the subject: and
b) administering an effective amount of the medicament to the subject, wherein the complex is substantially dissociated in the cell prior to administration.

4. The method of claim 1 comprising:
(a) selecting a medicament comprising an antibody against PTN on the basis of the antibody being capable of modulating the RPTP β/ζ signaling pathway in a cell of the subject, and
(b) administering an effective amount of the medicament to a subject in need thereof.

5. A method of cancer diagnosis in a subject, the method comprising determining the phosphorylation state of tyrosine-333 of β-catenin in a cell suspected of being cancerous, wherein the cell is obtained from the subject and wherein the cell is determined to be cancerous if tyrosine-333 of β-catenin is substantially phosphorylated in the cell.

6. A method of identifying an anti-cancer compound, the method comprising:
a) providing a cell in which β-catenin TYR-333 is substantially phosphorylated;
b) administering a candidate compound to the cell;
c) measuring the phosphorylation state of β-catenin-tyrosine-333 of the cell; and
d) determining that the candidate compound is an anti-cancer compound if the phosphorylation state of β-catenin-tyrosine-333 in the cell is decreased in the presence of the compound.

7. The method of claim 1 wherein the cancer, tumor or cell is from a cancer selected from the group consisting of adrenocortical carcinoma, AIDS-related cancers, AIDS-related lymphoma, anal cancer, appendix cancer, astrocytoma (childhood), cerebellar or cerebral, basal cell carcinoma, bile duct cancer, extrahepatic (bile duct) cancer, bladder cancer, bone cancer, osteosarcoma/malignant fibrous histiocytoma, brainstem glioma, cerebellar astrocytoma, cerebral astrocytoma/malignant glioma, ependymoma, medulloblastoma, supratentorial primitive neuroectodermal tumors, visual pathway and hypothalamic glioma, breast cancer (e.g., DCIS (Ductal Carcinoma In Situ), LCIS (Lobular Carcinoma In Situ), IDC (Invasive Ductal Carcinoma), e.g., scirrhous carcinoma, less common subtypes of Invasive Ductal Carcinoma (e.g., tubular carcinoma of the breast, medullary carcinoma of the breast, mucinous carcinoma of the breast, papillary carcinoma of the breast, and cribriform carcinoma of the breast), ILC (Invasive Lobular Carcinoma), Paget's Disease of the Nipple, Inflammatory Breast Cancer, Male Breast Cancer, Recurrent and Metastatic Breast Cancer, and phyllodes tumors, e.g., cystosarcoma phyllodes), bronchial adenomas/carcinoids, Burkitt lymphoma, carcinoid tumor (childhood), carcinoid tumor, gastrointestinal, carcinoma of unknown primary, central nervous system lymphoma (primary), cerebellar astrocytoma (childhood), cerebral astrocytoma/Malignant glioma (childhood), cervical cancer, childhood cancers, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor, extragonadal Germ cell tumor, extrahepatic bile duct cancer, eye cancer, intraocular melanoma, eye cancer, retinoblastoma, gallbladder cancer, gastric (stomach) cancer, gastrointestinal carcinoid tumor, gastrointestinal stromal tumor (GIST), germ cell tumor (extracranial), germ cell tumor (extragonadal), germ cell tumor (ovarian), gestational trophoblastic tumor, glioma (adult), glioma (childhood), childhood cerebral astrocytoma, glioma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma (childhood), intraocular melanoma, islet cell carcinoma (endocrine pancreas), Kaposi sarcoma, laryngeal cancer, hairy cell, lip and oral cavity cancer, liver cancer (primary), lung cancer (non-small cell), lung cancer (small cell), lymphomas, lymphoma (AIDS-related), lymphoma (Burkitt), lymphoma (cutaneous T-cell), lymphoma (Hodgkin), lymphomas (non-Hodgkin), lymphoma (primary central nervous system), macroglobulinemia, Waldenström, malignant fibrous histiocytoma of bone/osteosarcoma, medulloblastoma (childhood), melanoma, melanoma (intraocular (eye)), merkel cell carcinoma, mesothelioma (adult), mesothelioma (childhood), metastatic squamous neck cancer with occult primary, mouth cancer, multiple endocrine neoplasia syndrome (childhood), mycosis fungoides, myelodysplastic syndromes, myelodysplastic/myeloproliferative diseases, myelogenous leukemia (chronic), myeloid leukemia (adult acute), myeloid leukemia (childhood acute), myeloma (multiple (cancer of the bone-marrow)), myeloproliferative disorders (chronic), nasal cavity and paranasal sinus cancer, nasopharyngeal carcinoma, neuroblastoma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma (childhood), salivary gland cancer, sarcoma (soft tissue), sarcoma (uterine), Sézary syndrome, skin cancer (nonmelanoma), skin cancer (melanoma), skin carcinoma, Merkel cell, small intestine cancer, soft tissue sarcoma, squamous cell carcinoma, squamous neck cancer with occult primary, stomach cancer, supratentorial primitive neuroectodermal tumor (childhood), T-cell lymphoma, cutaneous (e.g., mycosis fungoides and Sézary syndrome), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site carcinoma, ureter and renal pelvis, transitional cell cancer, urethral cancer, uterine cancer, endometrial, uterine sarcoma, vaginal cancer, visual pathway and hypothalamic glioma (childhood), vulvar cancer, Waldenström macroglobulinemia, and Wilms tumor (kidney cancer).

8. The method of claim 7 wherein the cancer is breast cancer.

9. The method of claim 7 wherein the breast cancer is selected from the group consisting of DCIS (Ductal Carcinoma In Situ), LCIS (Lobular Carcinoma In Situ), IDC (Invasive Ductal Carcinoma), tubular carcinoma of the breast, medullary carcinoma of the breast, mucinous carcinoma of the breast, papillary carcinoma of the breast, cribriform carcinoma of the breast, ILC (Invasive Lobular Carcinoma), Paget's Disease of the Nipple, Inflammatory Breast Cancer, Male Breast Cancer, Recurrent and Metastatic Breast Cancer, and cystosarcoma phyllodes.

10. The method according to claim 1 wherein the method comprises administering an effective amount of a medicament comprising an antibody against PTN or a fragment thereof, in combination with negative PTN, decoy RPTP β/ζ, or combinations thereof.

11. (canceled)
